SEQUENCE LISTING (7 sequences)

SEQ ID NO: 1 - protein, 360 aa, synthetic construct
  REGION 1..360: note = Synthetic polymer
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 2 - protein, 317 aa, synthetic construct
  REGION 1..317: note = Synthetic polymer
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 3 - protein, 479 aa, synthetic construct
  REGION 1..479: note = Synthetic polymer
MRQPPGESDMAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 4 - protein, 454 aa, synthetic construct
  REGION 1..454: note = Synthetic polymer
MDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 5 - DNA, 846 bp, synthetic construct
  misc_feature 1..846: note = Synthetic polymer
  source 1..846: mol_type = other DNA
atggccatgatggaagtgcagggtgggccctccctcgggcagacgtgcgtgctcatagtcattttcaccgtgctcctccagagcctgtgcgtcgccgtcacgtacgtgtacttcaccaacgagctgaagcagatgcaggacaagtacagcaagtcggggatcgcctgcttcctgaaggaggacgactcctactgggacccgaacgacgaggagtccatgaactccccgtgctggcaggtcaagtggcagctgcggcagctggtccgcaagatgatcctccggaccagcgaggagactatcagcaccgtccaggagaagcagcagaacatctccccgctcgtccgggagcgaggtccccagcgggtagcggctcatatcaccggcacccgcgggaggtcgaacacgctcagctcgccgaactccaagaacgagaaggccctcggccgcaagatcaactcctgggagagctcgcggagcgggcactcgttcctgtcgaacctgcatctgcggaacggcgagctcgtgatccacgagaagggcttctactacatctacagccagacctacttccggttccaggaggagatcaaggagaacaccaagaacgacaagcagatggtgcagtacatctacaagtacaccagctaccccgacccgatcctcctgatgaagtccgcccgcaacagctgctggagcaaggacgcggagtacggcctgtacagcatctaccagggcggcatcttcgagctcaaggagaacgaccgcatcttcgtgagcgtgacgaacgagcacctgatcgacatggaccacgaggcctccttcttcggggcgttcctggtcggttga

SEQ ID NO: 6 - DNA, 708 bp, synthetic construct
  misc_feature 1..708: note = Synthetic polymer
  source 1..708: mol_type = other DNA
atgacagtgctggcgcccgcctggtcccccacaacctacctcctcctgctgctgttgctgtcgtcgggcctgagcgggacgcaggactgctcgttccagcacagcccgatctcctccgacttcgcggtcaagatccgggagctgagcgactacctgttgcaggactaccccgtcaccgtcgcctccaacctgcaggacgaggagctctgcggtggcttgtggcgtctggtgttggctcagcgctggatggagcggctcaagacggtcgcggggtccaagatgcagggcctgctcgagcgcgtgaacacggagatccacttcgtcaccaagtgcgccttccagccgccacccagctgcctgcggttcgtccagaccaacatctcccgcctcctgcaggagacatccgagcagctggtggcgctgaagccctggatcacgcgccagaacttctcccgatgcctggagctgcagtgccagcctgactcctcaaccctgccacctccatggagtccccggcctttggaggccaccgcgcccactgctccgcagcccccactcctcttgctactcctgctccccgtaggccttctactgctggccgcagcctggtgcctacattggcaaagaacgcggcgacgtacaccccgccctggggagcaggtgcccccagtccccagtccccaggacctgctgcttgtggagcactga

SEQ ID NO: 7 - DNA, 906 bp, synthetic construct
  misc_feature 1..906: note = Synthetic polymer
  source 1..906: mol_type = other DNA
atgttccacgtgtcgttccggtacatcttcggcctcccgccgctcatcctggtcctgctccccgtggcgtcctcggactgcgacatcgagggcaaggacgggaagcagtacgagtcggtgctgatggtgtccatcgaccagctgctcgactccatgaaggagatcgggtcgaactgcctcaacaacgagttcaacttcttcaagcgccacatctgcgacgcgaacaaggaggggatgttcctcttccgggcggcccgcaagctccggcagttcctcaagatgaacagcacgggggacttcgacctgcacctgctgaaggtgtcggagggcaccaccatcctcctcaactgcaccggccaggtcaagggccggaagcccgccgccctgggcgaggcgcagcccacgaagtccctcgaggagaacaagtccctcaaggagcagaagaagctcaacgacctgtgcttcctcaagcggctcctccaggagatcaagacctgctggaacaagatcctcatgggcaccaaggagcacgggggcggcgggagcggcgggggtggctccaactgggtgaacgtcatctcggacctcaagaagatcgaggacctgatccagagcatgcacatcgacgccacgctctacaccgagtcggacgtccacccgtcgtgcaaggtcaccgccatgaagtgcttcctcctggagctgcaggtcatctcgctggagtccggcgacgccagcatccacgacacggtcgagaacctcatcatcctcgcgaacaactccctcagctcgaacggcaacgtgacggagagcgggtgcaaggagtgcgaggagctggaggagaagaacatcaaggagttcctgcagagcttcgtgcacatcgtgcagatgttcatcaacaccagctga

(19) United States
(12) Patent Application Publication
(10) Pub. No.: US 2023/0193207 A1
(43) Pub. Date: Jun. 22, 2023

(54) MESENCHYMAL STEM CELL THERAPIES

(71) Applicant: Factor Bioscience Inc., Cambridge, MA (US)

(72) Inventors: Matthew Angel, Cambridge, MA (US); Christopher Rohde, Cambridge, MA (US); Jasmine Harris, Cambridge, MA (US)

(21) Appl. No.: 18/046,787

(22) Filed: Oct. 14, 2022

Related U.S. Application Data
(63) Continuation of application No. PCT/US2021/029613, filed on Apr. 28, 2021.
(60) Provisional application No. 63/016,626, filed on Apr. 28, 2020.

(51) Int. Cl. C12N 5/0775; A61P 35/00; A61P 11/00

(52) U.S. Cl. CPC C12N 5/0668; A61P 35/00; A61P 11/00; C12N 2506/45; C12N 2501/60

(57) ABSTRACT

Cell-based therapies based on mesenchymal stem cells (MSCs) are described.

### PRIORITY

The present application is a continuation of PCT/US2021/029613 filed Apr. 28, 2021. PCT/US2021/029613 claims priority to U.S. Provisional Application No. 63/016,626, filed on Apr. 28, 2020. The contents of each of which are herein incorporated by reference in their entireties

### FIELD

The present invention relates, in part, to cells, including mesenchymal stem cells (MSCs) and their use as therapeutic agents.

### DESCRIPTION OF THE TEXT FILE SUBMITTED ELECTRONICALLY

The instant application contains a Sequence Listing which has been submitted electronically in XML format and is hereby incorporated by reference in its entirety. Said XML copy, created on Oct. 14, 2022, is named 61057-716.301.xml and is 13,592 bytes in size.

### BACKGROUND

Mesenchymal stem cells (MSCs) show promise for use in various indications, e.g. immune-related diseases, regenerative medicine. MSCs beneficially have a high plasticity, ability to mediate inflammation and promote cell growth, cell differentiation and tissue repair by immunomodulation and immunosuppression.

Naturally occurring human MSCs are a rare subset of non-hematopoietic stem cells localized around the vasculature and trabeculae in the bone marrow. Over the past decade, bone marrow-derived mesenchymal stromal cells have been investigated therapeutically in a variety of clinical settings, including graft versus host disease, ischemic/non-ischemic cardiovascular disease, and ischemic stroke. Limitations with bone marrow-derived mesenchymal stromal cells include a declining number and differentiation potential of the cells with increasing donor age, the inconsistent quality of bone marrow-derived mesenchymal stromal cells products, and the invasiveness of the requisite bone marrow aspiration procedure.

Given the possibilities of MSCs, improved sources of this cellular therapeutic material are desired.

### SUMMARY

Accordingly, the present invention relates, in part, to methods of making and using MSCs that are derived from induced pluripotent stem cells (iPSCs), e.g., via mRNA-based reprogramming. Such methods, which include the step of assaying the MSC for a protein expression and/or secretion signature, yield superior cell populations and/or therapeutic effect. Further, such methods yield MSCs that demonstrate consistency among samples/batches, thus providing therapeutic reliability.

In one aspect, there is provided a method of making a composition comprising a therapeutic cell, comprising: (a) reprogramming an induced pluripotent stem cell (iPSC) into a mesenchymal stem cell (MSC), the reprogramming comprising contacting the iPSC with one or more synthetic RNA molecules encoding a reprogramming factor; (b) assaying the MSC for a protein secretion signature, the protein secretion signature comprising an increased secretion of one or more proteins selected from MIP-1 alpha, SDF-1 alpha, IL-27, LIF, IL-1 beta, IL-2, IL-5, IL-12p70, IL-13, IL-17A, IL-31, G-CSF/CSF-3, IFN-gamma, TNF-alpha, HGF, MCP-1, IL-9, bNGF, MIP-3 alpha, Gro-alpha/KC, IL-1alpha, IL-23, MMP-1, IL-18, M-CSF, IL-21, M-CSF, IL-21, CD40L, IL-22, VEGF-A, BLC, Tweak, ENA-78 (LIX), MCP-3, MIF, and Eotaxin-3 and/or a decreased secretion of one or more proteins selected from IL-6, IL-8, and IL-4, wherein the increased and/or decreased secretion is relative to a bone marrow-derived MSC; and (c) formulating the MSC substantially having the protein secretion signature for therapy.

In another aspect, there is provided a cell produced by the method described herein.

In another aspect, there is provided a method of treating an inflammatory and/or immunomodulatory disease or disorder, comprising: (a) obtaining a mesenchymal stem cell (MSC), the MSC having been obtained from reprogramming an induced pluripotent stem cell (iPSC), the reprogramming comprising contacting the iPSC with one or more synthetic RNA molecules encoding a reprogramming factor and having a protein secretion signature, the protein secretion signature comprising an increased secretion of one or more proteins selected from MIP-1 alpha, SDF-1 alpha, IL-27, LIF, IL-1 beta, IL-2, IL-5, IL-12p70, IL-13, IL-17A, IL-31, G-CSF/CSF-3, IFN-gamma, TNF-alpha, HGF, MCP-1, IL-9, bNGF, MIP-3 alpha, Gro-alpha/KC, IL-1alpha, IL-23, MMP-1, IL-18, M-CSF, IL-21, M-CSF, IL-21, CD40L, IL-22, VEGF-A, BLC, Tweak, ENA-78 (LIX), MCP-3, MIF, and Eotaxin-3 and/or a decreased secretion of one or more proteins selected from IL-6, IL-8, and IL-4, wherein the increased and/or decreased secretion is relative to a bone marrow-derived MSC; and (b) administering an effective amount of the MSC substantially having the protein secretion signature for therapy to a patient in need thereof.

In another aspect, there is provided a method of treating or mitigating a respiratory distress associated with an infection, comprising: (a) obtaining a mesenchymal stem cell (MSC), the MSC having been obtained from reprogramming an induced pluripotent stem cell (iPSC), the reprogramming comprising contacting the iPSC with one or more synthetic RNA molecules encoding a reprogramming factor and having a protein secretion signature, the protein secretion signature comprising an increased secretion of one or more proteins selected from MIP-1 alpha, SDF-1 alpha, IL-27, LIF, IL-1 beta, IL-2, IL-5, IL-12p70, IL-13, IL-17A, IL-31, G-CSF/CSF-3, IFN-gamma, TNF-alpha, HGF, MCP-1, IL-9, bNGF, MIP-3 alpha, Gro-alpha/KC, IL-1alpha, IL-23, MMP-1, IL-18, M-CSF, IL-21, M-CSF, IL-21, CD40L, IL-22, VEGF-A, BLC, Tweak, ENA-78 (LIX), MCP-3, MIF, and Eotaxin-3 and/or a decreased secretion of one or more proteins selected from IL-6, IL-8, and IL-4, wherein the increased and/or decreased secretion is relative to a bone marrow-derived MSC; and (b) administering an effective amount of the MSC substantially having the protein secretion signature for therapy to a patient in need thereof.

In another aspect, there is provided a method of treating acute respiratory distress syndrome (ARDS) associated with a SARS-CoV-2 infection, comprising: (a) obtaining a mesenchymal stem cell (MSC), the MSC having been obtained from reprogramming an induced pluripotent stem cell (iPSC), the reprogramming comprising contacting the iPSC with one or more synthetic RNA molecules encoding a reprogramming factor and having a protein secretion signature, the protein secretion signature comprising an increased secretion of one or more proteins selected from MIP-1 alpha, SDF-1 alpha, IL-27, LIF, IL-1 beta, IL-2, IL-5, IL-12p70, IL-13, IL-17A, IL-31, G-CSF/CSF-3, IFN-gamma, TNF-alpha, HGF, MCP-1, IL-9, bNGF, MIP-3 alpha, Gro-alpha/KC, IL-1alpha, IL-23, MMP-1, IL-18, M-CSF, IL-21, M-CSF, IL-21, CD40L, IL-22, VEGF-A, BLC, Tweak, ENA-78 (LIX), MCP-3, MIF, and Eotaxin-3 and/or a decreased secretion of one or more proteins selected from IL-6, IL-8, and IL-4, wherein the increased and/or decreased secretion is relative to a bone marrow-derived MSC; and (b) administering an effective amount of the MSC substantially having the protein secretion signature for therapy to a patient in need thereof.

In another aspect, there is provided a method of treating a cancer, comprising: (a) obtaining a mesenchymal stem cell (MSC), the MSC having been obtained from reprogramming an induced pluripotent stem cell (iPSC), the reprogramming comprising contacting the iPSC with one or more synthetic RNA molecules encoding a reprogramming factor and having a protein secretion signature, the protein secretion signature comprising an increased secretion of one or more proteins selected from MIP-1 alpha, SDF-1 alpha, IL-27, LIF, IL-1 beta, IL-2, IL-5, IL-12p70, IL-13, IL-17A, IL-31, G-CSF/CSF-3, IFN-gamma, TNF-alpha, HGF, MCP-1, IL-9, bNGF, MIP-3 alpha, Gro-alpha/KC, IL-1alpha, IL-23, MMP-1, IL-18, M-CSF, IL-21, M-CSF, IL-21, CD40L, IL-22, VEGF-A, BLC, Tweak, ENA-78 (LIX), MCP-3, MIF, and Eotaxin-3 and/or a decreased secretion of one or more proteins selected from IL-6, IL-8, and IL-4, wherein the increased and/or decreased secretion is relative to a bone marrow-derived MSC; and (b) administering an effective amount of the MSC substantially having the protein secretion signature for therapy to a patient in need thereof.

In embodiments, the iPSC is derived from a human and/or a subject who is not intended to receive the therapy. In embodiments, the iPSC is allogeneic to a patient intended to receive the therapy.

In embodiments, the MSC is characterized by low or reduced inflammation and/or immunogenicity. In embodiments, the MSC is self-renewing, multipotent, immune inhibitory, and/or suitable for in vitro expansion with substantial loss of immunosuppressive properties. In embodiments, the MSC reduces the proliferation, quantity, and/or activity of an immune cell, optionally selected from a T cell and NK cell. In embodiments, the MSC substantially expresses one or more of CD73, CD90, and CD105 and/or substantially does not express one or more of CD14, CD34 and CD45.

In embodiments, the MSC has been altered to reduce expression and/or activity of one or more MHC molecules. In embodiments, the alteration is enabled by gene editing. In embodiments, the MHC molecules are MHC class I molecules. In embodiments, the expression and/or activity of MHC class I molecules is reduced by gene editing human B2M gene (e.g. NCBI Reference Sequence: NG_012920). In embodiments, the alteration is enabled by gene editing. In embodiments, the MHC molecules are MHC class II molecules. In embodiments, the expression and/or activity of MHC class II molecules is reduced by gene editing the MHC II transactivator (CIITA) gene (e.g. NCBI Reference Sequence: NG_009628.1). In embodiments, the MSC has been altered to reduce expression and/or activity of MHC class I and MHC class II molecules. In embodiments, the expression and/or activity of MHC class I molecules is reduced by gene editing the B2M gene and the expression of MHC class II molecules is reduced by gene editing the CIITA gene.

In embodiments, the synthetic RNA molecule is mRNA, wherein the mRNA comprises none, or one or more of a non-canonical nucleotide. In embodiments, the synthetic RNA molecule is in vitro transcribed.

In embodiments, the reprogramming is non-viral. In embodiments, the reprogramming factor is one or more of Oct4, Sox2, Klf4, c-Myc, I-Myc, Tert, Nanog, and Lin28.

In embodiments, the composition is suitable for use in the treatment of various diseases, including infectious diseases, optionally selected from an infection with a pathogen, optionally selected from a bacterium, virus, fungus, or parasite. In embodiments, the pathogen is a virus. In embodiments, the virus is an influenza virus or a member of the Coronaviridae family. In embodiments, the virus is SARS-CoV-2, which has or has not caused COVID-19. In embodiments, the COVID-19 is characterized by one or more of fever, cough, shortness of breath, diarrhea, upper respiratory symptoms, lower respiratory symptoms, pneumonia, and respiratory distress.

In embodiments, the therapy prevents or mitigates development of acute respiratory distress syndrome (ARDS) in a patient when administered. In embodiments, the therapy improves oxygenation in a patient when administered. In embodiments, the therapy prevents or mitigates a transition from respiratory distress to cytokine imbalance in a patient when administered.

In embodiments, the therapy reverses or prevents a cytokine storm, which may or may not be in the lungs and/or systemically, in a patient when administered. In embodiments, the therapy reverses or prevents excessive production of one or more inflammatory cytokines in a patient when administered. In embodiments, the inflammatory cytokine is one or more of IL-6, IL-1, IL-1 receptor antagonist (IL-1ra), IL-2ra, IL-10, IL-18, TNFα, interferon-γ, CXCL10, and CCL7. In embodiments, the cell is primed with one or more molecules selected from lipopolysaccharide (LPS), poly(I:C), a TNF family member, TNF-α, an interferon, and interferon-γ. In embodiments, the cell is primed with one or more synthetic RNA molecules selected from, an mRNA molecule and an siRNA molecule. In embodiments, the mRNA molecule encodes one or more of an immunomodulatory protein, a homing ligand, and a homing ligand receptor. In embodiments, the cell is gene edited to overexpress one or more of an immunomodulatory protein, a homing ligand, and a homing ligand receptor.

In embodiments, the composition is formulated for one or more routes of administration, including infusion and injection. In embodiments, the infusion is intravenous infusion. In embodiments, the injection is intravenous injection.

In embodiments, the protein secretion signature is (a) increased secretion of about 5, or about 10, or about 15, or about 20, or about 25, or about 30, or about 35, or all of MIP-1 alpha, SDF-1 alpha, IL-27, LIF, IL-1 beta, IL-2, IL-5, IL-12p70, IL-13, IL-17A, IL-31, G-CSF/CSF-3, IFN-gamma, TNF-alpha, HGF, MCP-1, IL-9, bNGF, MIP-3 alpha, Gro-alpha/KC, IL-1alpha, IL-23, MMP-1, IL-18, M-CSF, IL-21, M-CSF, IL-21, CD40L, IL-22, VEGF-A, BLC, Tweak, ENA-78 (LIX), MCP-3, MIF, and Eotaxin-3, and/or (b) decreased secretion of IL-6, IL-8, and/or IL-4. In embodiments, the protein secretion signature is secretion of one, or two, or three, or four, or five, or six, or all of MIP-1 alpha, G-CSF/CSF-3, M-CSF, BLC, Tweak, ENA-78 (LIX), and MCP-3. In embodiments, the protein secretion signature is decreased secretion, relative to the IL-6 secretion by a bone marrow-derived MSC, of about 5, or about 10, or about 15, or about 20, or about 25, or about 30, or all of MIP-1 alpha, IL-27, LIF, IL-1 beta, IL-2, IL-5, IL-12p70, IL-13, IL-17A, IL-31, G-CSF/CSF-3, IFN-gamma, TNF-alpha, HGF, IL-9, bNGF, MIP-3 alpha, Gro-alpha/KC, IL-1alpha, IL-23, IL-18, M-CSF, IL-21, M-CSF, IL-21, CD40L, IL-22, BLC, ENA-78 (LIX), MCP-3, MIF, and Eotaxin-3.

### BRIEF DESCRIPTION OF DRAWINGS

FIG. 1 shows comparison of methods to generate the present MSCs and bone marrow-derived MSCs.

FIG. 2 shows a gene expression-based cell signature.

FIG. 3 shows a protein secretion-based cell signature. For each protein (X axis) the left bar is the present MSCs and the right bar is bone marrow-derived MSCs.

FIG. 4 shows the PaO₂/FiO₂ ratio of ARDS model sheep that received the present MSCs (top curve) versus control (bottom curve).

FIG. 5 shows the norepinephrine requirement in the ARDS model sheep that received the present MSCs (bottom curve) versus control (top curve).

FIGS. 6A-B show reduced lung vascular injury in the ARDS model sheep present MSCs (bottom curve) versus control (top curve).

FIGS. 7A-C show tissue bacterial count in the MSC treatment group, as compared to controls in the ARDS model sheep. In FIG. 7B and FIG. 7C, the left bar is the present MSCs and the right bar is control.

FIG. 8 shows cytokine expression in mesenchymal stem cells (MSCs) following mRNA electroporation. In each set of four bars, the conditions are, left to right TRAIL/TNFSF10, IL-7, IL-15, and Flt-3 ligand/FLT3L.

FIG. 9 shows TRAIL expression in MSCs following electroporation and freezing.

FIG. 10 shows TRAIL secretion following electroporation of iPSC-derived MSCs with RNA encoding TRAIL.

FIG. 11 shows cancer cell killing with MSC supernatant.

### DETAILED DESCRIPTION

The present invention is based, in part, on the discovery of methods for making high quality MSCs that are immunosuppressive and characterizable by a protein secretion and/or gene expression signature. Accordingly, in aspects, the present invention provides methods of obtaining and using MSCs that are consistently produced, with desirable properties, making their therapeutic use more likely to be successful.

In one aspect, there is provided a method of making a composition comprising a therapeutic cell, comprising: (a) reprogramming an induced pluripotent stem cell (iPSC) into a mesenchymal stem cell (MSC), the reprogramming comprising contacting the iPSC with one or more synthetic RNA molecules encoding a reprogramming factor; (b) assaying the MSC for a protein secretion signature, the protein secretion signature comprising an increased secretion of one or more proteins selected from MIP-1 alpha, SDF-1 alpha, IL-27, LIF, IL-1 beta, IL-2, IL-5, IL-12p70, IL-13, IL-17A, IL-31, G-CSF/CSF-3, IFN-gamma, TNF-alpha, HGF, MCP-1, IL-9, bNGF, MIP-3 alpha, Gro-alpha/KC, IL-1alpha, IL-23, MMP-1, IL-18, M-CSF, IL-21, M-CSF, IL-21, CD40L, IL-22, VEGF-A, BLC, Tweak, ENA-78 (LIX), MCP-3, MIF, and Eotaxin-3 and/or a decreased secretion of one or more proteins selected from IL-6, IL-8, and IL-4, wherein the increased and/or decreased secretion is relative to a bone marrow-derived MSC; and (c) formulating the MSC substantially having the protein secretion signature for therapy.

In another aspect, there is provided a cell produced by the method described herein.

In another aspect, there is provided a method of treating an inflammatory and/or immunomodulatory disease or disorder, comprising: (a) obtaining a mesenchymal stem cell (MSC), the MSC having been obtained from reprogramming an induced pluripotent stem cell (iPSC), the reprogramming comprising contacting the iPSC with one or more synthetic RNA molecules encoding a reprogramming factor and having a protein secretion signature, the protein secretion signature comprising an increased secretion of one or more proteins selected from MIP-1 alpha, SDF-1 alpha, IL-27, LIF, IL-1 beta, IL-2, IL-5, IL-12p70, IL-13, IL-17A, IL-31, G-CSF/CSF-3, IFN-gamma, TNF-alpha, HGF, MCP-1, IL-9, bNGF, MIP-3 alpha, Gro-alpha/KC, IL-1alpha, IL-23, MMP-1, IL-18, M-CSF, IL-21, M-CSF, IL-21, CD40L, IL-22, VEGF-A, BLC, Tweak, ENA-78 (LIX), MCP-3, MIF, and Eotaxin-3 and/or a decreased secretion of one or more proteins selected from IL-6, IL-8, and IL-4, wherein the increased and/or decreased secretion is relative to a bone marrow-derived MSC; and (b) administering an effective amount of the MSC substantially having the protein secretion signature for therapy to a patient in need thereof.

In another aspect, there is provided a method of treating or mitigating a respiratory distress associated with an infection, comprising: (a) obtaining a mesenchymal stem cell (MSC), the MSC having been obtained from reprogramming an induced pluripotent stem cell (iPSC), the reprogramming comprising contacting the iPSC with one or more synthetic RNA molecules encoding a reprogramming factor and having a protein secretion signature, the protein secretion signature comprising an increased secretion of one or more proteins selected from MIP-1 alpha, SDF-1 alpha, IL-27, LIF, IL-1 beta, IL-2, IL-5, IL-12p70, IL-13, IL-17A, IL-31, G-CSF/CSF-3, IFN-gamma, TNF-alpha, HGF, MCP-1, IL-9, bNGF, MIP-3 alpha, Gro-alpha/KC, IL-1alpha, IL-23, MMP-1, IL-18, M-CSF, IL-21, M-CSF, IL-21, CD40L, IL-22, VEGF-A, BLC, Tweak, ENA-78 (LIX), MCP-3, MIF, and Eotaxin-3 and/or a decreased secretion of one or more proteins selected from IL-6, IL-8, and IL-4, wherein the increased and/or decreased secretion is relative to a bone marrow-derived MSC; and (b) administering an effective amount of the MSC substantially having the protein secretion signature for therapy to a patient in need thereof.

In another aspect, there is provided a method of treating acute respiratory distress syndrome (ARDS), comprising: (a) obtaining a mesenchymal stem cell (MSC), the MSC having been obtained from reprogramming an induced pluripotent stem cell (iPSC), the reprogramming comprising contacting the iPSC with one or more synthetic RNA molecules encoding a reprogramming factor and having a protein secretion signature, the protein secretion signature comprising an increased secretion of one or more proteins selected from MIP-1 alpha, SDF-1 alpha, IL-27, LIF, IL-1 beta, IL-2, IL-5, IL-12p70, IL-13, IL-17A, IL-31, G-CSF/CSF-3, IFN-gamma, TNF-alpha, HGF, MCP-1, IL-9, bNGF, MIP-3 alpha, Gro-alpha/KC, IL-1alpha, IL-23, MMP-1, IL-18, M-CSF, IL-21, M-CSF, IL-21, CD40L, IL-22, VEGF-A, BLC, Tweak, ENA-78 (LIX), MCP-3, MIF, and Eotaxin-3 and/or a decreased secretion of one or more proteins selected from IL-6, IL-8, and IL-4, wherein the increased and/or decreased secretion is relative to a bone marrow-derived MSC; and (b) administering an effective amount of the MSC substantially having the protein secretion signature for therapy to a patient in need thereof. In one embodiment, the ARDS is associated with SARS-CoV-2 infection.

In aspects, there is provided a method of making a composition comprising a therapeutic cell, comprising formulating an MSC substantially having a desired protein secretion signature for therapy, the protein secretion signature comprising an increased secretion of one or more proteins selected from MIP-1 alpha, SDF-1 alpha, IL-27, LIF, IL-1 beta, IL-2, IL-5, IL-12p70, IL-13, IL-17A, IL-31, G-CSF/CSF-3, IFN-gamma, TNF-alpha, HGF, MCP-1, IL-9, bNGF, MIP-3 alpha, Gro-alpha/KC, IL-1alpha, IL-23, MMP-1, IL-18, M-CSF, IL-21, M-CSF, IL-21, CD40L, IL-22, VEGF-A, BLC, Tweak, ENA-78 (LIX), MCP-3, MIF, and Eotaxin-3 and/or a decreased secretion of one or more proteins selected from IL-6, IL-8, and IL-4, where the increased and/or decreased secretion is relative to a bone marrow-derived MSC and/or the MSC being derived from an induced pluripotent stem cell (iPSC) via reprogramming comprising contacting the iPSC with one or more synthetic RNA molecules encoding a reprogramming factor.

Induced Pluripotent Stem Cells (iPSCs) and Mesenchymal Stem Cells (MSCs)

Mature differentiated cells can be reprogrammed and dedifferentiated into embryonic-like cells, with embryonic stem cell-like properties. Fibroblast cells can be reversed into pluripotency via, for example, retroviral transduction of certain transcription factors, resulting in iPSCs. In some embodiments, iPSCs are generated from various tissues, including fibroblasts, keratinocytes, melanocyte blood cells, bone marrow cells, adipose cells, and tissue-resident progenitor cells. In some embodiments, iPSCs are generated via transduction of Oct3/4, Sox2, and Klf4.

The generation of iPSCs depends on the transduction of specific transcription factors into the somatic cell genome via vectors for its reprogramming.

In some embodiments, following iPSC generation, cells are assessed via pluripotency assays, including morphological and histological analysis, and certain gene expression profiles, proving their ability to differentiate into tissues derived from the three germ layers and teratoma formation. In some embodiments, teratoma assays involve injection of iPSCs into immunocompromised experimental animals and subsequent formed tissue analysis to assess teratoma formation.

In embodiments, the iPSC is derived from a human. In embodiments, the iPSC is derived from a subject who is not intended to receive the therapy. In embodiments, the iPSC is allogeneic to a patient intended to receive the therapy. In embodiments, the iPSC is from a master cell bank.

Stem cells have the ability to self-renew and differentiate into multiple cell types and so have applications in regenerative medicine. MSCs are multipotent adult stem cells that are capable of self-renewal and differentiation into cells of mesodermal lineage, such as bone, fat, and cartilage. In some embodiments, MSCs secrete factors that are proangiogenic, anti-apoptotic, and/or inhibit inflammation, that promote remodeling, and/or that release exosomes containing microRNAs that can influence disease associated processes.

MSCs are the most commonly used in cell therapies, because they are comparatively easy to obtain from a range of tissues and rarely undergo spontaneous differentiation during ex vivo expansion. In some embodiments, MSCs have anti-inflammatory properties and homing abilities to damaged sites. Human MSCs can be obtained from a variety of sources, including bone marrow and vascularized tissues throughout the body, including adipose tissue, placenta, amniotic fluid, dental pulp, synovial membrane, peripheral blood periodontal ligament, endometrium, and umbilical cord blood. However, the homogeneity, effectiveness, and differential potential of MSCs may differ depending on the source of the MSCs.

The use of autologous MSCs in therapeutic applications is safe because the cells will not elicit an immune response. However, it may be difficult to obtain a large amount of bone marrow or adipose tissue from the subject. Autologous MSCs may also have reduced therapeutic efficacy resulting in poor clinical outcomes. Additionally, if MSCs are needed urgently, there may not be time to extract and expand autologous MSCs from a subject.

Thus, the use of allogeneic MSCs is an attractive alternative, because donors can be prescreened for having cells with a high therapeutic potential. Allogeneic MSCs can be prepared on a clinical scale, assayed for therapeutic potential after production and stored in usable clinical doses that can be used readily for urgent therapeutic applications. Allogeneic MSCs inherently have low immunogenicity, because they lack or have reduced expression of MHC Class II antigens. In some embodiments, the present MSCs are allogeneic.

Thus, obtaining MSCs by reprogramming iPSCs, which may be obtained by reprogramming human somatic cells, is an attractive alternative. In some embodiments, iPSCs are obtained and MSCs are generated via cell reprogramming with non-immunogenic messenger RNA (mRNA) encoding one or more reprogramming factors in a defined, animal-component-free process.

In some embodiments, MSCs are checked for safety using one or more of bacterial and fungal tests, mycoplasma test, adventitious viral agent test, and tumorigenicity assay (karyotype analysis, teratoma formation assay, soft agar assay, comparative genomic hybridization (CGH), fluorescence in situ hybyridzation (FISH), and polymerase chain reaction (PCR)). In some embodiments, MSCs are assayed for their multilineage differentiation ability by testing the capability of MSCs to differentiate into osteoblasts, chondrocytes, and/or adipocytes.

In embodiments, the MSC is characterized by low or reduced inflammation. In embodiments, the MSC is characterized by low or reduced immunogenicity. In embodiments, the MSC is suitable for in vitro expansion without substantial loss of immunosuppressive properties. In embodiments, the MSC is self-renewing. In embodiments, the MSC is multipotent. In embodiments, the MSC is immune inhibitory.

In embodiments, the MSC has been altered to reduce expression of MHC molecules. In embodiments, the alteration is enabled by gene editing. In embodiments, the MHC molecules are MHC class I molecules. In embodiments, the expression of MHC class I molecules is reduced by gene editing the B2M gene. In embodiments, the alteration is enabled by gene editing. In embodiments, the MHC molecules are MHC class

II molecules. In embodiments, the expression of MHC class II molecules is reduced by gene editing the CIITA gene. In embodiments, the MSC has been altered to reduce expression of MHC class I and MHC class II molecules. In embodiments, the expression of MHC class I molecules is reduced by gene editing the B2M gene and the expression of MHC class II molecules is reduced by gene editing the CIITA gene.

In embodiments, the MSC reduces the proliferation, quantity, and/or activity of an immune cell, optionally selected from a T cell and NK cell.

In embodiments, the MSC substantially expresses one or more of CD73, CD90, and CD105. In embodiments, the MSC substantially does not express one or more of CD14, CD34 and CD45.

In some embodiments, MSCs are differentiated into adipocytes, osteoblasts, and chondrocytes.

Protein Expression and/or Secretion Signature

Each type of cell, including MSCs, expresses particular sets of proteins, within the cell, on the cell's surface, and secreted into the extracellular space. The particular sets of proteins that each type of cell expresses depends on the general and immediate function of the cell. Protein expression is correlated with mRNA levels and thus can be assayed by methods that analyze the distribution, amount, and identity of particular mRNAs within a cell. There are several methods of quantitatively measuring mRNA, including northern blotting and reverse transcription-quantitative PCR (RT-qPCR). Hybridization microarrays may also be used to generate expression profiles or high-throughput analyses of a range of genes within a cell. Further, ‘tag based’ technologies, such as Serial analysis of gene expression (SAGE) and RNA-Seq can be used to determine the relative measure of the cellular concentration of different mRNAs.

In some embodiments, protein expression of MSCs is determined by determining concentration of different mRNAs by one or more of northern blotting, RT-qPCR, hybridization microarrays, and tag based technologies, such as SAGE and RNA-Seq.

There are generally two strategies used for detection of proteins in the extracellular milieu: direct methods and indirect methods. The direct method comprises a one-step staining, and may involve a labeled antibody (e.g. FITC conjugated antiserum) reacting directly with the protein in the extracellular milieu. The indirect method comprises an unlabeled primary antibody that reacts with the protein in the extracellular milieu, and a labeled secondary antibody that reacts with the primary antibody. Labels can include radioactive labels, fluorescent labels, hapten labels such as, biotin, or an enzyme such as horse radish peroxidase or alkaline phosphatase. Methods of conducting these assays are well known in the art. See, e.g., Harlow et al. (Antibodies, Cold Spring Harbor Laboratory, N Y, 1988), Harlow et al. (Using Antibodies, A Laboratory Manual, Cold Spring Harbor Laboratory, N Y, 1999), Virella (Medical Immunology, 6th edition, Informa HealthCare, New York, 2007), and Diamandis et al. (Immunoassays, Academic Press, Inc., New York, 1996). Kits for conducting these assays are commercially available from, for example, Clontech Laboratories, LLC. (Mountain View, Calif.). In some embodiments, proteins are detected in the extracellular milieu of MSCs using detection methods comprising one or more antibodies. In some embodiments, the detection methods further comprise labels, including radioactive labels, fluorescent labels, hapten labels such as, biotin, or an enzyme such as horse radish peroxidase or alkaline phosphatase.

In some embodiments, flow cytometry is used to determine whether MSCs express certain sets of proteins that are on the surface or that are secreted. In some embodiments, antibodies specific to particular proteins are used in combination with proteomic approaches to determine, e.g., the protein secretion signature of an MSC. In some embodiments, the supernatant of a purified set of MSCs is assayed using a Western blot to determine the concentrations of an array of secreted proteins, to which antibodies are available. In some embodiments, the protein secretion signatures of MSCs derived from different sources, such as iPSCs or bone marrow are determined and compared.

In some embodiments, MSCs derived from bone marrow (BM-MSCs) and MSCs derived from iPSCs (iPSC-MSC) are separately purified and suspended in a serum-free culture medium at the same cell concentration, whereupon half of the BM-MSCs and half of the iPSC-MSCs are subjected to a protein expression assay of choice to determine the mRNA levels of a specific set of secreted proteins, while the other half of the BM-MSCs and the other half of the iPSC-MSCs are subjected to a Western blot analysis using antibodies to the specific set of secreted proteins, wherein the Western blot analysis enables the determination of a protein secretion signature for each of the BM-MSC population and the iPSC-MSC population with regard to the specific set of secreted proteins. In some embodiments, the results from the protein expression assay are used to determine the correlation of mRNA levels and protein secretion levels of MSCs. In some embodiments, control tests utilize the comparative analysis of (a) BM-MSCs derived on separate occasions from two different matches of bone marrow and compared to each other, and/or (b) iPSC-MSCs derived on separate occasions from different batches of iPSCs and compared to each other.

In embodiments, the protein secretion signature is increased secretion of about 5, or about 10, or about 15, or about 20, or about 25, or about 30, or about 35, or all of MIP-1 alpha, SDF-1 alpha, IL-27, LIF, IL-1 beta, IL-2, IL-5, IL-12p70, IL-13, IL-17A, IL-31, G-CSF/CSF-3, IFN-gamma, TNF-alpha, HGF, MCP-1, IL-9, bNGF, MIP-3 alpha, Gro-alpha/KC, IL-1alpha, IL-23, MMP-1, IL-18, M-CSF, IL-21, M-CSF, IL-21, CD40L, IL-22, VEGF-A, BLC, Tweak, ENA-78 (LIX), MCP-3, MIF, and Eotaxin-3.

In embodiments, the protein secretion signature is decreased secretion of IL-6, IL-8, and/or IL-4.

In embodiments, the protein secretion signature is (a) increased secretion of about 5, or about 10, or about 15, or about 20, or about 25, or about 30, or about 35, or all of MIP-1 alpha, SDF-1 alpha, IL-27, LIF, IL-1 beta, IL-2, IL-5, IL-12p70, IL-13, IL-17A, IL-31, G-CSF/CSF-3, IFN-gamma, TNF-alpha, HGF, MCP-1, IL-9, bNGF, MIP-3 alpha, Gro-alpha/KC, IL-1 alpha, IL-23, MMP-1, IL-18, M-CSF, IL-21, M-CSF, IL-21, CD40L, IL-22, VEGF-A, BLC, Tweak, ENA-78 (LIX), MCP-3, MIF, and Eotaxin-3, and (b) decreased secretion of IL-6, IL-8, and/or IL-4.

In embodiments, the protein secretion signature is secretion of one, or two, or three, or four, or five, or six, or all of MIP-1 alpha, G-CSF/CSF-3, M-CSF, BLC, Tweak, ENA-78 (LIX), and MCP-3.

In embodiments, the protein secretion signature is decreased secretion, relative to the IL-6 secretion by a bone marrow-derived MSC, of about 5, or about 10, or about 15, or about 20, or about 25, or about 30, or all of MIP-1 alpha, IL-27, LIF, IL-1 beta, IL-2, IL-5, IL-12p70, IL-13, IL-17A, IL-31, G-CSF/CSF-3, IFN-gamma, TNF-alpha, HGF, IL-9, bNGF, MIP-3 alpha, Gro-alpha/KC, IL-1alpha, IL-23, IL-18, M-CSF, IL-21, M-CSF, IL-21, CD40L, IL-22, BLC, ENA-78 (LIX), MCP-3, MIF, and Eotaxin-3.

In embodiments, the protein secretion signature is increased secretion of about 5, or about 10, or about 15, or about 20, or about 25, or about 30, or about 35, or all of:
1. MIP-1 alpha:
2. SDF-1 alpha, optionally about a 3-fold, or about a 4-fold, or about a 5-fold, or about a 6-fold, or about a 7-fold greater secretion than the secretion of SDF-1 alpha by a bone marrow-derived MSC;
3. IL-27, optionally about a 8-fold, or about a 9-fold, or about a 10-fold, or about a 11-fold, or about a 12-fold greater secretion than the secretion of IL-27 by a bone marrow-derived MSC;
4. LIF, optionally about a 1.5-fold, or about a 2-fold, or about a 2.5-fold, or about a 3-fold, or about a 3.5-fold greater secretion than the secretion of LIF by a bone marrow-derived MSC;
5. IL-1 beta, optionally about a 1.1-fold, or about a 1.3-fold, or about a 1.5-fold, or about a 1.7-fold, or about a 1.9-fold greater secretion than the secretion of IL-1 beta by a bone marrow-derived MSC;
6. IL-2, optionally about a 1.2-fold, or about a 1.6-fold, or about a 2-fold, or about a 2.4-fold, or about a 2.8-fold greater secretion than the secretion of IL-2 by a bone marrow-derived MSC;
7. IL-5, optionally about a 1.2-fold, or about a 1.5-fold, or about a 1.8-fold, or about a 2.1-fold, or about a 2.4-fold greater secretion than the secretion of IL-5 by a bone marrow-derived MSC;
8. IL-12p70, optionally about a 1.1-fold, or about a 1.3-fold, or about a 1.5-fold, or about a 1.7-fold, or about a 1.9-fold greater secretion than the secretion of IL-12p70 by a bone marrow-derived MSC;
9. IL-13, optionally about a 1.2-fold, or about a 1.3-fold, or about a 1.4-fold, or about a 1.5-fold, or about a 1.6-fold greater secretion than the secretion of IL-13 by a bone marrow-derived MSC;
10. IL-17A, optionally about a 4-fold, or about a 5-fold, or about a 6-fold, or about a 7-fold, or about a 8-fold greater secretion than the secretion of IL-17A by a bone marrow-derived MSC;
11. IL-31, optionally about a 4-fold, or about a 5-fold, or about a 6-fold, or about a 7-fold, or about a 8-fold greater secretion than the secretion of IL-31 by a bone marrow-derived MSC;
12. G-CSF/CSF-3;
13. IFN-gamma, optionally about a 1.5-fold, or about a 2-fold, or about a 2.5-fold, or about a 3-fold, or about a 3.5-fold greater secretion than the secretion of IFN-gamma by a bone marrow-derived MSC;
14. TNF-alpha, optionally about a 1.2-fold, or about a 1.6-fold, or about a 2-fold, or about a 2.4-fold, or about a 2.8-fold greater secretion than the secretion of TNF-alpha by a bone marrow-derived MSC;
15. HGF, optionally about a 2-fold, or about a 3-fold, or about a 4-fold, or about a 5-fold, or about a 6-fold greater secretion than the secretion of HGF by a bone marrow-derived MSC;
16. MCP-1, optionally about a 2-fold, or about a 3-fold, or about a 4-fold, or about a 5-fold, or about a 6-fold greater secretion than the secretion of MCP-1 by a bone marrow-derived MSC;
17. IL-9, optionally about a 1.1-fold, or about a 1.2-fold, or about a 1.3-fold, or about a 1.4-fold, or about a 1.5-fold greater secretion than the secretion of IL-9 by a bone marrow-derived MSC;
18. bNGF, optionally about a 6-fold, or about a 7-fold, or about a 8-fold, or about a 9-fold, or about a 10-fold greater secretion than the secretion of bNGF by a bone marrow-derived MSC;
19. MIP-3 alpha, optionally about a 2-fold, or about a 3-fold, or about a 4-fold, or about a 5-fold, or about a 6-fold greater secretion than the secretion of MIP-3 alpha by a bone marrow-derived MSC;
20. Gro-alpha/KC, optionally about a 5-fold, or about a 6-fold, or about a 7-fold, or about a 8-fold, or about a 9-fold greater secretion than the secretion of Gro-alpha/KC by a bone marrow-derived MSC;
21. IL-1alpha, optionally about a 11-fold, or about a 12-fold, or about a 13-fold, or about a 14-fold, or about a 15-fold greater secretion than the secretion of IL-1alpha by a bone marrow-derived MSC;
22. IL-23, optionally about a 5-fold, or about a 6-fold, or about a 7-fold, or about a 8-fold, or about a 9-fold greater secretion than the secretion of IL-23 by a bone marrow-derived MSC;
23. MMP-1, optionally about a 14-fold, or about a 16-fold, or about a 18-fold, or about a 20-fold, or about a 22-fold greater secretion than the secretion of MMP-1 by a bone marrow-derived MSC;
24. IL-18, optionally about a 1.5-fold, or about a 2-fold, or about a 2.5-fold, or about a 3-fold, or about a 3.5-fold greater secretion than the secretion of IL-18 by a bone marrow-derived MSC;
25. M-CSF;
26. IL-21, optionally about a 1.2-fold, or about a 1.6-fold, or about a 2-fold, or about a 2.4-fold, or about a 2.8-fold greater secretion than the secretion of IL-21 by a bone marrow-derived MSC;
27. IL-3, optionally about a 2-fold, or about a 3-fold, or about a 4-fold, or about a 5-fold, or about a 6-fold greater secretion than the secretion of IL-3 by a bone marrow-derived MSC;
28. CD40L, optionally about a 1.1-fold, or about a 1.15-fold, or about a 1.2-fold, or about a 1.25-fold, or about a 1.3-fold greater secretion than the secretion of CD40L by a bone marrow-derived MSC;
29. IL-22, optionally about a 1.2-fold, or about a 1.6-fold, or about a 2-fold, or about a 2.4-fold, or about a 2.8-fold greater secretion than the secretion of IL-22 by a bone marrow-derived MSC;
30. VEGF-A, optionally about a 3-fold, or about a 4-fold, or about a 5-fold, or about a 6-fold, or about a 7-fold greater secretion than the secretion of VEGF-A by a bone marrow-derived MSC;
31. BLC;
32. Tweak;
33. ENA-78 (LIX);
34. MCP-3;
35. MIF, optionally about a 1.2-fold, or about a 1.3-fold, or about a 1.4-fold, or about a 1.5-fold, or about a 1.6-fold greater secretion than the secretion of MIF by a bone marrow-derived MSC; and
36. Eotaxin-3, optionally about a 1.3-fold, or about a 1.4-fold, or about a 1.5-fold, or about a 1.6-fold, or about a 1.7-fold greater secretion than the secretion of Eotaxin-3 by a bone marrow-derived MSC.

In embodiments, the protein secretion signature is a lesser secretion of about 5, or about 10, or about 15, or about 20, or about 25, or about 30, or all of the following, relative to the secretion of IL-6 by a bone marrow-derived MSC. In embodiments, the protein secretion signature is a lesser secretion of about 5, or about 10, or about 15, or about 20, or about 25, or about 30, or all of the following:
1. MIP-1 alpha, optionally about 160-fold, or about 170-fold, or about 180-fold, or about 190-fold, or about 200-fold, less than the secretion of IL-6 a bone marrow-derived MSC;
2. IL-27, optionally about 55-fold, or about 60-fold, or about 65-fold, or about 70-fold, or about 75-fold, less than the secretion of IL-6 a bone marrow-derived MSC;
3. LIF, optionally about 2-fold, or about 3-fold, or about 4-fold, or about 5-fold, or about 6-fold, less than the secretion of IL-6 a bone marrow-derived MSC;
4. IL-1 beta, optionally about 210-fold, or about 230-fold, or about 250-fold, or about 270-fold, or about 290-fold, less than the secretion of IL-6 a bone marrow-derived MSC;
5. IL-2, optionally about 11-fold, or about 13-fold, or about 15-fold, or about 17-fold, or about 19-fold, less than the secretion of IL-6 a bone marrow-derived MSC;
6. IL-5, optionally about 35-fold, or about 40-fold, or about 45-fold, or about 50-fold, or about 55-fold, less than the secretion of IL-6 a bone marrow-derived MSC;
7. IL-6, optionally about 2-fold, or about 3-fold, or about 4-fold, or about 5-fold, or about 6-fold, less than the secretion of IL-6 a bone marrow-derived MSC;
8. IL-8, optionally about 14-fold, or about 16-fold, or about 18-fold, or about 20-fold, or about 22-fold, less than the secretion of IL-6 a bone marrow-derived MSC;
9. IL-4, optionally about 40-fold, or about 45-fold, or about 50-fold, or about 55-fold, or about 60-fold, less than the secretion of IL-6 a bone marrow-derived MSC;
10. IL-12p70, optionally about 210-fold, or about 230-fold, or about 250-fold, or about 270-fold, or about 290-fold, less than the secretion of IL-6 a bone marrow-derived MSC;
11. IL-13, optionally about 130-fold, or about 140-fold, or about 150-fold, or about 160-fold, or about 170-fold, less than the secretion of IL-6 a bone marrow-derived MSC;
12. IL-17A, optionally about 20-fold, or about 25-fold, or about 30-fold, or about 35-fold, or about 40-fold, less than the secretion of IL-6 a bone marrow-derived MSC;
13. IL-31, optionally about 25-fold, or about 30-fold, or about 35-fold, or about 40-fold, or about 45-fold, less than the secretion of IL-6 in a bone marrow-derived MSC;
14. G-CSF/CSF-3, optionally about 80-fold, or about 90-fold, or about 100-fold, or about 110-fold, or about 120-fold, less than the secretion of IL-6 a bone marrow-derived MSC;
15. IFN-gamma, optionally about 30-fold, or about 35-fold, or about 40-fold, or about 45-fold, or about 50-fold, less than the secretion of IL-6 in a bone marrow-derived MSC;
16. TNF-alpha, optionally about 100-fold, or about 110-fold, or about 120-fold, or about 130-fold, or about 140-fold, less than the secretion of IL-6 a bone marrow-derived MSC;
17. HGF, optionally about 2.5-fold, or about 3-fold, or about 3.5-fold, or about 4-fold, or about 4.5-fold, less than the secretion of IL-6 a bone marrow-derived MSC;
18. IL-9, optionally about 110-fold, or about 120-fold, or about 130-fold, or about 140-fold, or about 150-fold, less than the secretion of IL-6 a bone marrow-derived MSC;
19. bNGF, optionally about 40-fold, or about 45-fold, or about 50-fold, or about 55-fold, or about 60-fold, less than the secretion of IL-6 a bone marrow-derived MSC;
20. MIP-3 alpha, optionally about 2-fold, or about 2.5-fold, or about 3-fold, or about 3.5-fold, or about 4-fold, less than the secretion of IL-6 a bone marrow-derived MSC;
21. Gro-alpha/KC, optionally about 3-fold, or about 3.5-fold, or about 4-fold, or about 4.5-fold, or about 5-fold, less than the secretion of IL-6 a bone marrow-derived MSC;
22. IL-1alpha, optionally about 45-fold, or about 50-fold, or about 55-fold, or about 60-fold, or about 65-fold, less than the secretion of IL-6 a bone marrow-derived MSC;
23. IL-23, optionally about 65-fold, or about 70-fold, or about 75-fold, or about 80-fold, or about 85-fold, less than the secretion of IL-6 a bone marrow-derived MSC;
24. IL-18, optionally about 20-fold, or about 22.5-fold, or about 25-fold, or about 27.5-fold, or about 30-fold, less than the secretion of IL-6 a bone marrow-derived MSC;
25. M-CSF, optionally about 4.5-fold, or about 5-fold, or about 5.5-fold, or about 6-fold, or about 6.5-fold, less than the secretion of IL-6 a bone marrow-derived MSC;
26. IL-21, optionally about 4-fold, or about 4.5-fold, or about 5-fold, or about 5.5-fold, or about 6-fold, less than the secretion of IL-6 a bone marrow-derived MSC;
27. IL-3, optionally about 15-fold, or about 17.5-fold, or about 20-fold, or about 22.5-fold, or about 25-fold, less than the secretion of IL-6 a bone marrow-derived MSC;
28. CD40L, optionally about 20-fold, or about 22.5-fold, or about 25-fold, or about 27.5-fold, or about 30-fold, less than the secretion of IL-6 a bone marrow-derived MSC;
29. IL-22, optionally about 9-fold, or about 10-fold, or about 11-fold, or about 12-fold, or about 13-fold, less than the secretion of IL-6 a bone marrow-derived MSC;
30. BLC, optionally about 6-fold, or about 7-fold, or about 8-fold, or about 9-fold, or about 10-fold, less than the secretion of IL-6 a bone marrow-derived MSC;
31. ENA-78 (LIX), optionally about 1.2-fold, or about 1.3-fold, or about 1.4-fold, or about 1.5-fold, or about 1.6-fold, less than the secretion of IL-6 a bone marrow-derived MSC;
32. MCP-3, optionally about 12-fold, or about 13-fold, or about 14-fold, or about 15-fold, or about 16-fold, less than the secretion of IL-6 a bone marrow-derived MSC;
33. MIF, optionally about 14-fold, or about 15-fold, or about 16-fold, or about 17-fold, or about 18-fold, less than the secretion of IL-6 a bone marrow-derived MSC; and
34. Eotaxin-3, optionally about 24-fold, or about 26-fold, or about 28-fold, or about 30-fold, or about 32-fold, less than the secretion of IL-6 a bone marrow-derived MSC.

In embodiments, the protein secretion signature is as shown in Table 1. In embodiments, any of the values of Table 1 may vary by about 5%, or about 10%, or about 15% in the context of the protein secretion signature.

TABLE 1

| Sample | MIP-1 alpha | SDF-1 alpha | IL-27 | LIF | IL-1beta | IL-2 | IL-5 |
|---|---|---|---|---|---|---|---|
| Present MSCs | 2.2 | 894.3 | 6.0 | 90.9 | 1.6 | 27.4 | 8.5 |
| BM-MSCs | 0 | 162.3 | 0.6 | 35.4 | 1.1 | 14.1 | 4.7 |
| Fold Change (Present MSCs vs. BM) | | 5.5 | 10.6 | 2.6 | 1.5 | 1.9 | 1.8 |
| Fold Less (Present MSCs vs. BM IL-6) | 183.4 | 0.4 | 65.7 | 4.4 | 247.4 | 14.5 | 46.8 |
| Present MSCs vs. BM relative to IL-6 | 0.022 | 9.1 | 0.061 | 0.920 | 0.016 | 0.28 | 0.086 |
| BM MSC relative to IL-6 | 0.0 | 0.41 | 0.0014 | 0.089 | 0.0028 | 0.036 | 0.012 |
| Fold change, normalized to IL-6 | | 22.1 | 42.6 | 10.3 | 5.9 | 7.8 | 7.2 |

| Sample | IL-6 | IL-8 | IL-4 | IL-12p70 | IL-13 | IL-17A | IL-31 |
|---|---|---|---|---|---|---|---|
| Present MSCs | 98.8 | 21.0 | 7.7 | 1.6 | 2.6 | 12.8 | 11.6 |
| BM-MSCs | 397.0 | 25.1 | 9.5 | 1.1 | 1.9 | 2.0 | 2.1 |
| Fold Change (Present MSCs vs. BM) | 0.2 | 0.8 | 0.8 | 1.5 | 1.4 | 6.3 | 5.5 |
| Fold Less (Present MSCs vs. BM IL-6) | 4.0 | 18.9 | 51.7 | 248.2 | 153.2 | 31.0 | 34.2 |
| Present MSCs vs. BM relative to IL-6 | 1.00 | 0.21 | 0.078 | 0.016 | 0.026 | 0.13 | 0.12 |
| BM MSC relative to IL-6 | 1.00 | 0.063 | 0.024 | 0.0027 | 0.0048 | 0.0051 | 0.0053 |
| Fold change, normalized to IL-6 | 1.0 | 3.4 | 3.2 | 6.1 | 5.5 | 25.3 | 22.1 |

| Sample | G-CSF/CSF-3 | IFN-gamma | TNF-alpha | HGF | MCP-1 | IL-9 | bNGF |
|---|---|---|---|---|---|---|---|
| Present MSCs | 4.1 | 10.1 | 3.3 | 110.5 | 398.1 | 2.9 | 8.3 |
| BM-MSCs | 0.0 | 3.9 | 1.9 | 28.0 | 93.8 | 2.3 | 1.1 |
| Fold Change (Present MSCs vs. BM) | | 2.6 | 1.8 | 4.0 | 4.2 | 1.3 | 7.6 |
| Fold Less (Present MSCs vs. BM IL-6) | 97.7 | 39.4 | 118.7 | 3.6 | 1.0 | 134.9 | 48.0 |
| Present MSCs vs. BM relative to IL-6 | 0.041 | 0.102 | 0.034 | 1.12 | 4.03 | 0.030 | 0.084 |
| BM MSC relative to IL-6 | 0.0 | 0.0098 | 0.0047 | 0.070 | 0.24 | 0.0058 | 0.0027 |
| Fold change, normalized to IL-6 | | 10.4 | 7.2 | 15.9 | 17.1 | 5.1 | 30.7 |

| Sample | MIP-3 alpha | Gro-alpha/KC | IL-1alpha | IL-23 | MMP-1 | IL-18 | M-CSF |
|---|---|---|---|---|---|---|---|
| Present MSCs | 127.2 | 95.1 | 7.0 | 5.1 | 6541.9 | 15.6 | 74.7 |
| BM-MSCs | 35.3 | 13.8 | 0.5 | 0.7 | 347.3 | 5.9 | 0.0 |
| Fold Change (Present MSCs vs. BM) | 3.6 | 6.9 | 13.0 | 7.3 | 18.8 | 2.6 | |
| Fold Less (Present MSCs vs. BM IL-6) | 3.1 | 4.2 | 56.6 | 77.6 | 0.1 | 25.4 | 5.3 |
| Present MSCs vs. BM relative to IL-6 | 1.3 | 0.96 | 0.071 | 0.052 | 66.2 | 0.16 | 0.76 |
| BM MSC relative to IL-6 | 0.089 | 0.035 | 0.0014 | 0.0018 | 0.87 | 0.015 | 0.0 |
| Fold change, normalized to IL-6 | 14.5 | 27.7 | 52.1 | 29.2 | 75.7 | 10.6 | |

| Sample | IL-21 | IL-3 | CD40L | IL-22 | VEGF-A | BLC |
|---|---|---|---|---|---|---|
| Present MSCs | 78.8 | 19.3 | 14.8 | 34.8 | 14985.6 | 47.1 |
| BM-MSCs | 40.6 | 4.4 | 12.4 | 16.5 | 3080.2 | 0.0 |
| Fold Change (Present MSCs vs. BM) | 1.9 | 4.3 | 1.2 | 2.1 | 4.9 | |
| Fold Less (Present MSCs vs. BM IL-6) | 5.0 | 20.6 | 26.8 | 11.4 | 0.0 | 8.4 |
| Present MSCs vs. BM relative to IL-6 | 0.80 | 0.20 | 0.15 | 0.35 | 151.7 | 0.48 |
| BM MSC relative to IL-6 | 0.102 | 0.011 | 0.031 | 0.042 | 7.8 | 0.0 |
| Fold change, normalized to IL-6 | 7.8 | 17.5 | 4.8 | 8.4 | 19.6 | |

| Sample | Tweak | ENA-78 (LIX) | MCP-3 | MIF | Eotaxin-3 |
|---|---|---|---|---|---|
| Present MSCs | 406.9 | 281.0 | 28.1 | 24.7 | 14.0 |
| BM-MSCs | 0.0 | 0.0 | 0.0 | 17.6 | 9.5 |
| Fold Change (Present MSCs vs. BM) | | | | 1.4 | 1.5 |
| Fold Less (Present MSCs vs. BM IL-6) | 1.0 | 1.4 | 14.1 | 16.0 | 28.3 |
| Present MSCs vs. BM relative to IL-6 | 4.1 | 2.8 | 0.28 | 0.25 | 0.14 |
| BM MSC relative to IL-6 | 0.0 | 0.0 | 0.0 | 0.044 | 0.024 |
| Fold change, normalized to IL-6 | | | | 5.7 | 5.9 |

### Reprogramming Methods and RNA

In various embodiments, the present invention relates to the reprogramming of iPSCs to MSCs, using non-viral, RNA-based means. iPSCs, namely pluripotent or less differentiated cells, can be reprogrammed from non-pluripotent or differentiated cells, including fibroblasts, keratinocytes, melanocyte blood cells, bone marrow cells, adipose cells, and tissue-resident progenitor cells.

In some embodiments, the method for reprogramming a non-pluripotent cell comprises: (a) providing a non-pluripotent cell; (b) culturing the non-pluripotent cell; and (c) transfecting the non-pluripotent cell with one or more synthetic RNA molecules, wherein the one or more synthetic RNA molecules include at least one RNA molecule encoding one or more reprogramming factors selected from the group consisting of Oct4 protein, Sox2 protein, Klf4 protein, c-Myc protein, I-Myc protein, Tert protein, Nanog protein, and Lin28 protein; wherein the transfecting results in the cell expressing the one or more reprogramming factors to result in the cell being reprogrammed; and wherein step (c) occurs in the presence of a medium containing ingredients that support reprogramming of the differentiated cell to a less differentiated state.

In some embodiments, the method for reprogramming a differentiated cell to a less differentiated state, comprises: (a) providing a differentiated cell; (b) culturing the differentiated cell; and (c) transfecting the differentiated cell with one or more synthetic RNA molecules, wherein the one or more synthetic RNA molecules include at least one RNA molecule encoding one or more reprogramming factors selected from the group consisting of Oct4 protein, Sox2 protein, Klf4 protein, c-Myc protein, I-Myc protein, Tert protein, Nanog protein, and Lin28 protein; wherein the transfecting results in the cell expressing the one or more reprogramming factors to result in the cell being reprogrammed to a less differentiated state; and wherein step (c) occurs in the presence of a medium containing ingredients that support reprogramming of the differentiated cell to a less differentiated state.

In some embodiments, the method for reprogramming a differentiated cell to a less differentiated state, comprises: (a) providing a differentiated cell; (b) culturing the differentiated cell; and (c) transfecting the differentiated cell with one or more synthetic RNA molecules, wherein the one or more synthetic RNA molecules include at least one RNA molecule encoding one or more reprogramming factors; wherein the transfecting results in the cell expressing the one or more reprogramming factors; and wherein step (c) is performed at least twice and the amount of one or more synthetic RNA molecules transfected in one or more later transfections is greater than the amount transfected in one or more earlier transfections to result in the cell being reprogrammed to a less differentiated state and occurs in the presence of a medium containing ingredients that support reprogramming of the differentiated cell to a less differentiated state.

In some embodiments, the method for reprogramming a non-pluripotent cell, comprises: (a) providing a non-pluripotent cell; (b) culturing the non-pluripotent cell; and (c) transfecting the non-pluripotent cell with one or more synthetic RNA molecules, wherein the one or more synthetic RNA molecules include at least one RNA molecule encoding one or more reprogramming factors; wherein the transfecting results in the cell expressing the one or more reprogramming factors to result in the cell being reprogrammed; and wherein step (c) is performed without using irradiated human neonatal fibroblast feeder cells and occurs in the presence of a medium containing ingredients that support reprogramming of the cell.

In some embodiments, the method for reprogramming a differentiated cell to a less differentiated state, comprises: (a) providing a differentiated cell; (b) culturing the differentiated cell; and (c) transfecting the differentiated cell with one or more synthetic RNA molecules, wherein the one or more synthetic RNA molecules include at least one RNA molecule encoding one or more reprogramming factors; wherein the transfecting results in the cell expressing the one or more reprogramming factors to result in the cell being reprogrammed to a less differentiated state; and wherein step (c) is performed without using irradiated human neonatal fibroblast feeder cells and occurs in the presence of a medium containing ingredients that support reprogramming of the cell to a less differentiated state.

In some embodiments, the method for reprogramming a non-pluripotent cell, comprises: (a) providing a non-pluripotent cell; (b) culturing the non-pluripotent cell; (c) transfecting the non-pluripotent cell with one or more synthetic RNA molecules, wherein the one or more synthetic RNA molecules include at least one RNA molecule encoding one or more reprogramming factors and wherein the transfecting results in the cell expressing the one or more reprogramming factors; and (d) repeating step (c) at least twice during 5 consecutive days, wherein the amount of one or more synthetic RNA molecules transfected in one or more later transfections is greater than the amount transfected in one or more earlier transfections, to result in the non-pluripotent cell being reprogrammed, wherein steps (c) and (d) occur in the presence of a medium containing ingredients that support reprogramming of the non-pluripotent cell.

In some embodiments, the method for reprogramming a differentiated cell to a less differentiated state, comprises: (a) providing a differentiated cell; (b) culturing the differentiated cell; (c) transfecting the differentiated cell with one or more synthetic RNA molecules, wherein the one or more synthetic RNA molecules include at least one RNA molecule encoding one or more reprogramming factors and wherein the transfecting results in the cell expressing the one or more reprogramming factors; and (d) repeating step (c) at least twice during 5 consecutive days, wherein the amount of one or more synthetic RNA molecules transfected in one or more later transfections is greater than the amount transfected in one or more earlier transfections, to result in the cell being reprogrammed to a less differentiated state, wherein steps (c) and (d) occur in the presence of a medium containing ingredients that support reprogramming of the differentiated cell to a less differentiated state.

In some embodiments, the method for reprogramming a non-pluripotent cell comprises: (a) providing a non-pluripotent cell, the non-pluripotent cell being derived from a biopsy of a human subject; (b) culturing the non-pluripotent cell; and (c) transfecting the non-pluripotent cell with a synthetic RNA molecule, wherein: the synthetic RNA molecule encodes one or more reprogramming factor(s) selected from the group consisting of Oct4 protein, Sox2 protein, Klf4 protein, c-Myc protein, I-Myc protein, Tert protein, Nanog protein, and Lin28 protein, the transfecting results in the non-pluripotent cell expressing the one or more reprogramming factor(s) which reprograms the non-pluripotent cell; and step (c) is performed without using irradiated human neonatal fibroblast feeder cells and occurs in the presence of a medium containing ingredients that support reprogramming of the non-pluripotent cell.

In some embodiments, the method for reprogramming a cell to a less differentiated state, comprises: (a) providing a non-pluripotent cell; (b) culturing the cell; and (c) transfecting the cell with a synthetic RNA molecule, wherein: the RNA molecule encodes one or more reprogramming factor(s) selected from the group consisting of Oct4 protein, Sox2 protein, Klf4 protein, c-Myc protein, I-Myc protein, Tert protein,

Nanog protein, and Lin28 protein, the transfecting results in the cell expressing the one or more reprogramming factor(s) which reprograms the cell to a less differentiated state, and step (c) is performed without using irradiated human neonatal fibroblast feeder cells and occurs in the presence of a medium containing ingredients that support reprogramming of the cell to a less differentiated state.

In some embodiments, the method for reprogramming a cell to a less differentiated state, comprises: (a) providing a non-pluripotent cell; (b) culturing the cell in a medium containing ingredients that support reprogramming of the cell to a less differentiated state; and (c) transfecting the cell with a synthetic RNA molecule, wherein: the RNA molecule encodes one or more reprogramming factor(s) selected from the group consisting of Oct4 protein, Sox2 protein, Klf4 protein, c-Myc protein, I-Myc protein, Tert protein, Nanog protein, and Lin28 protein, the transfecting results in the cell expressing the one or more reprogramming factor(s) which reprograms the cell to a less differentiated state, and step (c) is performed without using irradiated human neonatal fibroblast feeder cells and occurs in the presence of a feeder cell conditioned medium.

In some embodiments, the method for reprogramming a cell to a less differentiated state comprises: (a) providing a non-pluripotent cell; (b) culturing the cell in a medium containing albumin and ingredients that support reprogramming of the cell to a less differentiated state, wherein the albumin is treated with an ion-exchange resin or charcoal; (c) transfecting the cell with a synthetic RNA molecule, wherein the RNA molecule encoding one or more reprogramming factor(s) selected from the group consisting of Oct4 protein, Sox2 protein, Klf4 protein, c-Myc protein, I-Myc protein, Tert protein, Nanog protein, and Lin28 protein, wherein the transfecting results in the cell expressing the one or more reprogramming factor(s) which reprograms the cell to a less differentiated state.

In some embodiments, the method for reprogramming a cell to a less differentiated state, comprises: (a) culturing a differentiated cell with a reprogramming medium; (b) transfecting the cell with one or more synthetic RNA molecules, wherein the one or more synthetic RNA molecules include at least one RNA molecule encoding one or more reprogramming factors and wherein the transfecting results in the cell expressing the one or more reprogramming factors; and (c) repeating step (b) at least twice during 5 consecutive days, wherein the amount of one or more synthetic RNA molecules transfected in one or more later transfections is greater than the amount transfected in one or more earlier transfections, to result in the cell being reprogrammed to a less differentiated state, wherein steps (a)-(c) are performed without using feeder cells and occur in the presence of a feeder cell conditioned medium.

In some embodiments, the method for reprogramming a cell to a less differentiated state, comprises: a. culturing a differentiated cell with a reprogramming medium containing albumin, wherein the albumin is treated with an ion-exchange resin or charcoal; b. transfecting the cell with one or more synthetic RNA molecules, wherein the one or more synthetic RNA molecules includes at least one RNA molecule encoding one or more reprogramming factors and wherein the transfecting results in the cell expressing the one or more reprogramming factors; and c. repeating step (b) at least twice during 5 consecutive days to result in the cell being reprogrammed to a less differentiated state.

In some embodiments, the method for reprogramming a cell to a less differentiated state, comprises: a. culturing a differentiated cell with a reprogramming medium containing albumin, wherein the albumin is treated with sodium octanoate; brought to a temperature of at least about 40° C.; and treated with an ion-exchange resin or charcoal; b. transfecting the cell with one or more synthetic RNA molecules, wherein the one or more synthetic RNA molecules includes at least one RNA molecule encoding one or more reprogramming transcription factors and wherein the transfecting results in the cell expressing the one or more synthetic RNA molecules; and c. repeating step (b) at least twice during about 5 consecutive days to result in the cell being reprogrammed to a less differentiated state.

In embodiments, the reprogramming is non-viral. In embodiments, the reprogramming factor is one or more of Oct4, Sox2, Klf4, c-Myc, I-Myc, Tert, Nanog, and Lin28.

In some embodiments, iPSCs are obtained and MSCs are generated via cell reprogramming with non-immunogenic messenger RNA (mRNA) encoding one or more reprogramming factors in a defined, animal component-free process. In some embodiments the process is immunosuppressant-free. In some embodiments, the process is animal component-free. In some embodiments, the process is defined.

In some embodiments, iPSCs are generated from adult human dermal fibroblasts using a high-efficiency, immunosuppressant-free mRNA-based protocol, whereupon iPSCs are then differentiated into MSCs using a 21-day high-yield monolayer protocol. In some embodiments, the differentiated MSCs are characterized by downregulation of Nanog and Oct4 and/or upregulation of CD73 and CD105, e.g., relative to the source cells. In some embodiments, rtPCR analysis is used to characterize MSCs. In some embodiments, multipotency of MSCs is confirmed by differentiation into adipocytes, osteoblasts, and chondrocytes. In some embodiments, the present MSCs are characterized by having approximately 13 kb long telomeres compared to 7 kb long telomeres of bone marrow-derived MSCs (BM MSCs), as measured by Southern analysis of terminal restriction fragments.

In some embodiments, upon serial passaging, the present MSCs undergo >70 population doublings before senescence, compared to <20 population doublings for BM MSCs. In some embodiments, the present MSCs are capable of undergoing greater than about 25, or greater than about 30, or greater than about 35, or greater than about 40, or greater than about 45, or greater than about 50, or greater than about 55, or greater than about 60, or greater than about 65, or greater than about 70, or greater than about 75 doublings before senescence.”

Cells can be reprogrammed by exposing them to specific extracellular cues and/or by ectopic expression of specific proteins, microRNAs, etc. While several reprogramming methods have been previously described, most that rely on ectopic expression require the introduction of exogenous DNA, which can carry mutation risks. DNA-free reprogramming methods based on direct delivery of reprogramming proteins have been reported. However, these methods are too inefficient and unreliable for commercial use. In addition, RNA-based reprogramming methods have been described (see, e.g., Angel. MIT Thesis. 2008. 1-56; Angel et al. PLoS ONE. 2010. 5, 107; Warren et al. Cell Stem Cell. 2010. 7, 618-630; Angel. MIT Thesis. 2011. 1-89; and Lee et al., Cell. 2012. 151, 547-558; the contents of all of which are hereby incorporated by reference). However, existing RNA-based reprogramming methods are slow, unreliable, and inefficient when performed on adult cells, require many transfections (resulting in significant expense and opportunity for error), can reprogram only a limited number of cell types, can reprogram cells to only a limited number of cell types, require the use of immunosuppressants, and require the use of multiple human-derived components, including blood-derived HSA and human fibroblast feeders. The many drawbacks of previously disclosed RNA-based reprogramming methods make them undesirable for research, therapeutic or cosmetic use.

In some embodiments, reprogramming is performed by transfecting cells with one or more nucleic acids encoding one or more reprogramming factors, including, but not limited to Oct4 protein, Sox2 protein, Klf4 protein, c-Myc protein, I-Myc protein, TERT protein, Nanog protein, Lin28 protein, Utf1 protein, Aicda protein, miR200 micro-RNA, miR302 micro-RNA, miR367 micro-RNA, miR369 micro-RNA and biologically active fragments, analogues, variants and family-members thereof. In some embodiments, reprogramming the cell is performed in vivo. In one embodiment, the cell in vivo is reprogrammed by transfecting the cell with one or more nucleic acids encoding one or more reprogramming factors. In one embodiment, the one or more nucleic acids includes an RNA molecule that encodes Oct4 protein. In another embodiment, the one or more nucleic acids also includes one or more RNA molecules that encodes Sox2 protein, Klf4 protein, and c-Myc protein. In yet another embodiment, the one or more nucleic acids also includes an RNA molecule that encodes Lin28 protein. In one embodiment, the cell is a human skin cell, and the human skin cell is reprogrammed to a pluripotent stem cell. In another embodiment, the cell is a human skin cell, and the human skin cell is reprogrammed to a glucose-responsive insulin-producing cell. Examples of other cells that can be reprogrammed and other cells to which a cell can be reprogrammed include, but are not limited to skin cells, pluripotent stem cells, MSCs, β-cells, retinal pigmented epithelial cells, hematopoietic cells, cardiac cells, airway epithelial cells, neural stem cells, neurons, glial cells, bone cells, blood cells, and dental pulp stem cells. In one embodiment, the cell is contacted with a medium that supports the reprogrammed cell. In one embodiment, the medium also supports the cell.

Importantly, infecting skin cells with viruses encoding Oct4, Sox2, Klf4, and c-Myc, combined with culturing the cells in a medium that supports the growth of cardiomyocytes, has been reported to cause reprogramming of the skin cells to cardiomyocytes, without first reprogramming the skin cells to pluripotent stem cells (See Efs et al Nat Cell Biol. 2011; 13:215-22, the contents of which are hereby incorporated by reference). In certain situations, direct reprogramming (reprogramming one somatic cell to another somatic cell without first reprogramming the somatic cell to a pluripotent stem cell, also known as “transdifferentiation”) may be desirable, in part because culturing pluripotent stem cells can be time-consuming and expensive, the additional handling involved in establishing and characterizing a stable pluripotent stem cell line can carry an increased risk of contamination, and the additional time in culture associated with first producing pluripotent stem cells can carry an increased risk of genomic instability and the acquisition of mutations, including point mutations, copy-number variations, and karyotypic abnormalities. Certain embodiments are therefore directed to a method for reprogramming a somatic cell in vivo, wherein the cell is reprogrammed to a somatic cell, and wherein a characterized pluripotent stem-cell line is not produced.

In embodiments, fewer total transfections may be required to reprogram a cell according to the methods of the present invention than according to other methods. Certain embodiments are therefore directed to a method for reprogramming a cell in vivo, wherein between about 1 and about 12 transfections are performed during about 20 consecutive days, or between about 4 and about 10 transfections are performed during about 15 consecutive days, or between about 4 and about 8 transfections are performed during about 10 consecutive days. It is recognized that when a cell is contacted with a medium containing nucleic acid molecules, the cell may likely come into contact with and/or internalize more than one nucleic acid molecule either simultaneously or at different times. A cell can therefore be contacted with a nucleic acid more than once, e.g. repeatedly, even when a cell is contacted only once with a medium containing nucleic acids.

Of note, nucleic acids can contain one or more non-canonical or “modified” residues as described herein. For instance, any of the non-canonical nucleotides described herein can be used in the present reprogramming methods. In one embodiment, pseudouridine-5′-triphosphate can be substituted for uridine-5′-triphosphate in an in vitro-transcription reaction to yield synthetic RNA, wherein up to 100% of the uridine residues of the synthetic RNA may be replaced with pseudouridine residues. In vitro-transcription can yield RNA with residual immunogenicity, even when pseudouridine and 5-methylcytidine are completely substituted for uridine and cytidine, respectively (see, e.g., Angel. Reprogramming Human Somatic Cells to Pluripotency Using RNA [Doctoral Thesis]. Cambridge, Mass.: MIT; 2011, the contents of which are hereby incorporated by reference). For this reason, it is common to add an immunosuppressant to the transfection medium when transfecting cells with RNA. In certain situations, adding an immunosuppressant to the transfection medium may not be desirable, in part because the recombinant immunosuppressant most commonly used for this purpose, B18R, can be expensive and difficult to manufacture. In embodiments, cells in vivo are transfected and/or reprogrammed according to the methods of the present invention, without using B18R or any other immunosuppressant. In embodiments, reprogramming cells in vivo according to the methods of the present invention without using immunosuppressants can be rapid, efficient, and reliable. Certain embodiments are therefore directed to a method for transfecting a cell in vivo, wherein the transfection medium does not contain an immunosuppressant. Other embodiments are directed to a method for reprogramming a cell in vivo, wherein the transfection medium does not contain an immunosuppressant. In certain situations, for example when using a high cell density, it may be beneficial to add an immunosuppressant to the transfection medium. Certain embodiments are therefore directed to a method for transfecting a cell in vivo, wherein the transfection medium contains an immunosuppressant. Other embodiments are directed to a method for reprogramming a cell in vivo, wherein the transfection medium contains an immunosuppressant. In one embodiment, the immunosuppressant is B18R or a biologically active fragment, analogue, variant or family-member thereof or dexamethasone or a derivative thereof. In one embodiment, the transfection medium does not contain an immunosuppressant, and the nucleic-acid dose is chosen to prevent excessive toxicity. In another embodiment, the nucleic-acid dose is less than about 1 mg/cm² of tissue or less than about 1 mg/100,000 cells or less than about 10 mg/kg.

Reprogrammed cells produced according to certain embodiments of the present invention are suitable for therapeutic and/or cosmetic applications as they do not contain undesirable exogenous DNA sequences, and they are not exposed to animal-derived or human-derived products, which may be undefined, and which may contain toxic and/or pathogenic contaminants. Furthermore, the high speed, efficiency, and reliability of certain embodiments of the present invention may reduce the risk of acquisition and accumulation of mutations and other chromosomal abnormalities. Certain embodiments of the present invention can thus be used to generate cells that have a safety profile adequate for use in therapeutic and/or cosmetic applications. For example, reprogramming cells using RNA and the medium of the present invention, wherein the medium does not contain animal or human-derived components, can yield cells that have not been exposed to allogeneic material. Certain embodiments are therefore directed to a reprogrammed cell that has a desirable safety profile. In one embodiment, the reprogrammed cell has a normal karyotype. In another embodiment, the reprogrammed cell has fewer than about 5 copy-number variations (CNVs) relative to the patient genome, such as fewer than about 3 copy-number variations relative to the patient genome, or no copy-number variations relative to the patient genome. In yet another embodiment, the reprogrammed cell has a normal karyotype and fewer than about 100 single nucleotide variants in coding regions relative to the patient genome, or fewer than about 50 single nucleotide variants in coding regions relative to the patient genome, or fewer than about 10 single nucleotide variants in coding regions relative to the patient genome.

Endotoxins and nucleases can co-purify and/or become associated with other proteins, such as serum albumin. Recombinant proteins, in particular, can often have high levels of associated endotoxins and nucleases, due in part to the lysis of cells that can take place during their production. Endotoxins and nucleases can be reduced, removed, replaced or otherwise inactivated by many of the methods of the present invention, including, for example, by acetylation, by addition of a stabilizer such as sodium octanoate, followed by heat treatment, by the addition of nuclease inhibitors to the albumin solution and/or medium, by crystallization, by contacting with one or more ion-exchange resins, by contacting with charcoal, by preparative electrophoresis or by affinity chromatography. In embodiments, partially or completely reducing, removing, replacing, or otherwise inactivating endotoxins and/or nucleases from a medium and/or from one or more components of a medium is provided and this can increase the efficiency with which cells can be transfected and reprogrammed. Certain embodiments are therefore directed to a method for transfecting a cell in vivo with one or more nucleic acids, wherein the transfection medium is treated to partially or completely reduce, remove, replace or otherwise inactivate one or more endotoxins and/or nucleases. Other embodiments are directed to a medium that causes minimal degradation of nucleic acids. In one embodiment, the medium contains less than about 1 EU/mL, or less than about 0.1 EU/mL, or less than about 0.01 EU/mL.

In certain situations, protein-based lipid carriers such as serum albumin can be replaced with non-protein-based lipid carriers such as methyl-beta-cyclodextrin. The medium of the present invention can also be used without a lipid carrier, for example, when transfection is performed using a method that may not require or may not benefit from the presence of a lipid carrier, for example, using one or more lipid-based transfection reagents, polymer-based transfection reagents or peptide-based transfection reagents or using electroporation. Many protein-associated molecules, such as metals, can be highly toxic to cells in vivo. This toxicity can cause decreased viability, as well as the acquisition of mutations. Certain embodiments thus have the additional benefit of producing cells that are free from toxic molecules.

The associated-molecule component of a protein can be measured by suspending the protein in solution and measuring the conductivity of the solution. Certain embodiments are therefore directed to a medium that contains a protein, wherein about a 10% solution of the protein in water has a conductivity of less than about 500 μmho/cm. In one embodiment, the solution has a conductivity of less than about 50 μmho/cm. In another embodiment, less than about 0.65% of the dry weight of the protein comprises lipids and/or less than about 0.35% of the dry weight of the protein comprises free fatty acids.

The amount of nucleic acid delivered to cells in vivo can be increased to increase the desired effect of the nucleic acid. However, increasing the amount of nucleic acid delivered to cells in vivo beyond a certain point can cause a decrease in the viability of the cells, due in part to toxicity of the transfection reagent. In embodiments, a nucleic acid is delivered to a population of cells in vivo in a fixed volume (for example, cells in a region of tissue), and the amount of nucleic acid delivered to each cell can depend on the total amount of nucleic acid delivered to the population of cells and to the density of the cells, with a higher cell density resulting in less nucleic acid being delivered to each cell. In certain embodiments, a cell in vivo is transfected with one or more nucleic acids more than once. Under certain conditions, for example when the cells are proliferating, the cell density may change from one transfection to the next. Certain embodiments are therefore directed to a method for transfecting a cell in vivo with a nucleic acid, wherein the cell is transfected more than once, and wherein the amount of nucleic acid delivered to the cell is different for two of the transfections. In one embodiment, the cell proliferates between two of the transfections, and the amount of nucleic acid delivered to the cell is greater for the second of the two transfections than for the first of the two transfections. In another embodiment, the cell is transfected more than twice, and the amount of nucleic acid delivered to the cell is greater for the second of three transfections than for the first of the same three transfections, and the amount of nucleic acid delivered to the cells is greater for the third of the same three transfections than for the second of the same three transfections. In yet another embodiment, the cell is transfected more than once, and the maximum amount of nucleic acid delivered to the cell during each transfection is sufficiently low to yield at least about 80% viability for at least two consecutive transfections.

In embodiments, there are provided methods in which modulating the amount of nucleic acid delivered to a population of proliferating cells in vivo in a series of transfections can result in both an increased effect of the nucleic acid and increased viability of the cells. In embodiments, when cells in vivo are contacted with one or more nucleic acids encoding one or more reprogramming factors in a series of transfections, the efficiency of reprogramming can be increased when the amount of nucleic acid delivered in later transfections is greater than the amount of nucleic acid delivered in earlier transfections, for at least part of the series of transfections. Certain embodiments are therefore directed to a method for reprogramming a cell in vivo, wherein one or more nucleic acids is repeatedly delivered to the cell in a series of transfections, and the amount of the nucleic acid delivered to the cell is greater for at least one later transfection than for at least one earlier transfection. In one embodiment, the cell is transfected between about 2 and about 10 times, or between about 3 and about 8 times, or between about 4 and about 6 times. In another embodiment, the one or more nucleic acids includes at least one RNA molecule, the cell is transfected between about 2 and about 10 times, and the amount of nucleic acid delivered to the cell in each transfection is the same as or greater than the amount of nucleic acid delivered to the cell in the most recent previous transfection. In yet another embodiment, the amount of nucleic acid delivered to the cell in the first transfection is between about 20 ng/cm² and about 250 ng/cm², or between 100 ng/cm² and 600 ng/cm². In yet another embodiment, the cell is transfected about 5 times at intervals of between about 12 and about 48 hours, and the amount of nucleic acid delivered to the cell is about 25 ng/cm² for the first transfection, about 50 ng/cm² for the second transfection, about 100 ng/cm² for the third transfection, about 200 ng/cm² for the fourth transfection, and about 400 ng/cm² for the fifth transfection. In yet another embodiment, the cell is further transfected at least once after the fifth transfection, and the amount of nucleic acid delivered to the cell is about 400 ng/cm².

Certain embodiments are directed to a method for transfecting a cell in vivo with a nucleic acid, wherein the amount of nucleic acid is determined by measuring the cell density, and choosing the amount of nucleic acid to transfect based on the measurement of cell density. In one embodiment, the cell density is measured by optical means. In another embodiment, the cell is transfected repeatedly, the cell density increases between two transfections, and the amount of nucleic acid transfected is greater for the second of the two transfections than for the first of the two transfections.

In embodiments, the in vivo transfection efficiency and viability of cells contacted with the medium of the present invention can be improved by conditioning the medium. Certain embodiments are therefore directed to a method for conditioning a medium. Other embodiments are directed to a medium that is conditioned. In one embodiment, the feeders are fibroblasts, and the medium is conditioned for approximately 24 hours. Other embodiments are directed to a method for transfecting a cell in vivo, wherein the transfection medium is conditioned. Other embodiments are directed to a method for reprogramming a cell in vivo, wherein the medium is conditioned. In one embodiment, the feeders are mitotically inactivated, for example, by exposure to a chemical such as mitomycin-C or by exposure to gamma radiation. In certain embodiments, it may be beneficial to use only autologous materials, in part to, for example and not wishing to be bound by theory, avoid the risk of disease transmission from the feeders to the cell or the patient. Certain embodiments are therefore directed to a method for transfecting a cell in vivo, wherein the transfection medium is conditioned, and wherein the feeders are derived from the same individual as the cell being transfected. Other embodiments are directed to a method for reprogramming a cell in vivo, wherein the medium is conditioned, and wherein the feeders are derived from the same individual as the cell being reprogrammed.

Several molecules can be added to media by conditioning. Certain embodiments are therefore directed to a medium that is supplemented with one or more molecules that are present in a conditioned medium. In one embodiment, the medium is supplemented with Wnt1, Wnt2, Wnt3, Wnt3a or a biologically active fragment, analogue, variant, agonist, or family-member thereof. In another embodiment, the medium is supplemented with TGF-β or a biologically active fragment, analogue, variant, agonist, or family-member thereof. In yet another embodiment, a cell in vivo is reprogrammed according to the method of the present invention, wherein the medium is not supplemented with TGF-β for between about 1 and about 5 days, and is then supplemented with TGF-β for at least about 2 days. In yet another embodiment, the medium is supplemented with IL-6, IL-6R or a biologically active fragment, analogue, variant, agonist, or family-member thereof. In yet another embodiment, the medium is supplemented with a sphingolipid or a fatty acid. In still another embodiment, the sphingolipid is lysophosphatidic acid, lysosphingomyelin, sphingosine-1-phosphate or a biologically active analogue, variant or derivative thereof.

In addition to mitotically inactivating cells, under certain conditions, irradiation can change the gene expression of cells, causing cells to produce less of certain proteins and more of certain other proteins than non-irradiated cells, for example, members of the Wnt family of proteins. In addition, certain members of the Wnt family of proteins can promote the growth and transformation of cells. In embodiments, the efficiency of reprogramming can be greatly increased by contacting a cell in vivo with a medium that is conditioned using irradiated feeders instead of mitomycin-c-treated feeders. In embodiments, the increase in reprogramming efficiency observed when using irradiated feeders is caused in part by Wnt proteins that are secreted by the feeders. Certain embodiments are therefore directed to a method for reprogramming a cell in vivo, wherein the cell is contacted with Wnt1, Wnt2, Wnt3, Wnt3a or a biologically active fragment, analogue, variant, family-member or agonist thereof, including agonists of downstream targets of Wnt proteins, and/or agents that mimic one or more of the biological effects of Wnt proteins, for example, 2-amino-4-[3,4-(methylenedioxy)benzylamino]-6-(3-methoxyphenyl)pyrimidine.

Because of the low efficiency of many DNA-based reprogramming methods, these methods may be difficult or impossible to use with cells derived from patient samples, which may contain only a small number of cells. In contrast, the high efficiency of certain embodiments of the present invention can allow reliable reprogramming of a small number of cells, including single cells. Certain embodiments are directed to a method for reprogramming a small number of cells. Other embodiments are directed to a method for reprogramming a single cell. In one embodiment, the cell is contacted with one or more enzymes. In another embodiment, the enzyme is collagenase. In yet another embodiment, the collagenase is animal-component free. In one embodiment, the collagenase is present at a concentration of between about 0.1 mg/mL and about 10 mg/mL, or between about 0.5 mg/mL and about 5 mg/mL. In another embodiment, the cell is a blood cell. In yet another embodiment, the cell is contacted with a medium containing one or more proteins that is derived from the patient's blood. In still another embodiment, the cell is contacted with a medium comprising: DMEM/F12+2 mM L-alanyl-L-glutamine+between about 5% and about 25% patient-derived serum, or between about 10% and about 20% patient-derived serum, or about 20% patient-derived serum.

In embodiments, transfecting cells in vivo with a mixture of RNA encoding Oct4, Sox2, Klf4, and c-Myc using the medium of the present invention can cause the rate of proliferation of the cells to increase. When the amount of RNA delivered to the cells is too low to ensure that all of the cells are transfected, only a fraction of the cells may show an increased proliferation rate. In certain situations, such as when generating a personalized therapeutic, increasing the proliferation rate of cells may be desirable, in part because doing so can reduce the time necessary to generate the therapeutic, and therefore can reduce the cost of the therapeutic. Certain embodiments are therefore directed to a method for transfecting a cell in vivo with a mixture of RNA encoding Oct4, Sox2, Klf4, and c-Myc. In one embodiment, the cell exhibits an increased proliferation rate. In another embodiment, the cell is reprogrammed.

While detailed examples are provided herein for the production of specific types of cells and for the production of therapeutics comprising specific types of cells, it is recognized that the methods of the present invention can be used to produce many other types of cells, and to produce therapeutics comprising one or more of many other types of cells, for example, by reprogramming a cell according to the methods of the present invention, and culturing the cell under conditions that mimic one or more aspects of development by providing conditions that resemble the conditions present in the cellular microenvironment during development.

Other embodiments are directed to a method for reprogramming a cell in vivo. In one embodiment, the cell is reprogrammed by contacting the cell with one or more nucleic acids. In one embodiment, the cell is contacted with a plurality of nucleic acids encoding at least one of Oct4 protein, Sox2 protein, Klf4 protein, c-Myc protein, Lin28 protein or a biologically active fragment, variant or derivative thereof. In another embodiment, the cell is contacted with a plurality of nucleic acids encoding a plurality of proteins including: Oct4 protein, Sox2 protein, Klf4 protein, and c-Myc protein or one or more biologically active fragments, variants or derivatives thereof.

Illustrative subjects or patients refers to any vertebrate including, without limitation, humans and other primates (e.g., chimpanzees and other apes and monkey species), farm animals (e.g., cattle, sheep, pigs, goats, and horses), domestic mammals (e.g., dogs and cats), laboratory animals (e.g., rodents such as mice, rats, and guinea pigs), and birds (e.g., domestic, wild and game birds such as chickens, turkeys and other gallinaceous birds, ducks, geese, and the like). In some embodiments, the subject is a mammal. In some embodiments, the subject is a human.

In some embodiments, a synthetic RNA molecule is used to reprogram iPSCs into MSCs. In embodiments, the synthetic RNA molecule is mRNA. In embodiments, the synthetic RNA molecule is in vitro transcribed.

In some embodiments, the synthetic RNA molecule contains one or more non-canonical nucleotides that include one or more substitutions at the 2C and/or 4C and/or 5C positions in the case of a pyrimidine or the 6C and/or 7N and/or 8C positions in the case of a purine can be less toxic than synthetic RNA molecules containing only canonical nucleotides, due in part to the ability of substitutions at these positions to interfere with recognition of synthetic RNA molecules by proteins that detect exogenous nucleic acids, and furthermore, that substitutions at these positions can have minimal impact on the efficiency with which the synthetic RNA molecules can be translated into protein, due in part to the lack of interference of substitutions at these positions with base-pairing and base-stacking interactions.

In embodiments, the synthetic RNA molecule is mRNA comprising one or more non-canonical nucleotides selected from 2-thiouridine, 5-azauridine, pseudouridine, 4-thiouridine, 5-methyluridine, 5-methylpseudouridine, 5-aminouridine, 5-aminopseudouridine, 5-hydroxyuridine, 5-hydroxypseudouridine, 5-methoxyuridine, 5-methoxypseudouridine, 5-ethoxyuridine, 5-ethoxypseudouridine, 5-hydroxymethyluridine, 5-hydroxymethylpseudouridine, 5-carboxyuridine, 5-carboxypseudouridine, 5-formyluridine, 5-formylpseudouridine, 5-methyl-5-azauridine, 5-amino-5-azauridine, 5-hydroxy-5-azauridine, 5-methylpseudouridine, 5-aminopseudouridine, 5-hydroxypseudouridine, 4-thio-5-azauridine, 4-thiopseudouridine, 4-thio-5-methyluridine, 4-thio-5-aminouridine, 4-thio-5-hydroxyuridine, 4-thio-5-methyl-5-azauridine, 4-thio-5-amino-5-azauridine, 4-thio-5-hydroxy-5-azauridine, 4-thio-5-methylpseudouridine, 4-thio-5-aminopseudouridine, 4-thio-5-hydroxypseudouridine, 2-thiocytidine, 5-azacytidine, pseudoisocytidine, N4-methylcytidine, N4-aminocytidine, N4-hydroxycytidine, 5-methylcytidine, 5-aminocytidine, 5-hydroxycytidine, 5-methoxycytidine, 5-ethoxycytidine, 5-hydroxymethylcytidine, 5-carboxycytidine, 5-formylcytydine, 5-methyl-5-azacytidine, 5-amino-5-azacytidine, 5-hydroxy-5-azacytidine, 5-methylpseudoisocytidine, 5-aminopseudoisocytidine, 5-hydroxypseudoisocytidine, N4-methyl-5-azacytidine, N4-methylpseudoisocytidine, 2-thio-5-azacytidine, 2-thiopseudoisocytidine, 2-thio-N4-methylcytidine, 2-thio-N4-aminocytidine, 2-thio-N4-hydroxycytidine, 2-thio-5-methylcytidine, 2-thio-5-aminocytidine, 2-thio-5-hydroxycytidine, 2-thio-5-methyl-5-azacytidine, 2-thio-5-amino-5-azacytidine, 2-thio-5-hydroxy-5-azacytidine, 2-thio-5-methylpseudoisocytidine, 2-thio-5-aminopseudoisocytidine, 2-thio-5-hydroxypseudoisocytidine, 2-thio-N4-methyl-5-azacytidine, 2-thio-N4-methylpseudoisocytidine, N4-methyl-5-methylcytidine, N4-methyl-5-aminocytidine, N4-methyl-5-hydroxycytidine, N4-methyl-5-methyl-5-azacytidine, N4-methyl-5-amino-5-azacytidine, N4-methyl-5-hydroxy-5-azacytidine, N4-methyl-5-methylpseudoisocytidine, N4-methyl-5-aminopseudoisocytidine, N4-methyl-5-hydroxypseudoisocytidine, N4-amino-5-azacytidine, N4-aminopseudoisocytidine, N4-amino-5-methylcytidine, N4-amino-5-aminocytidine, N4-amino-5-hydroxycytidine, N4-amino-5-methyl-5-azacytidine, N4-amino-5-amino-5-azacytidine, N4-amino-5-hydroxy-5-azacytidine, N4-amino-5-methylpseudoisocytidine, N4-amino-5-aminopseudoisocytidine, N4-amino-5-hydroxypseudoisocytidine, N4-hydroxy-5-azacytidine, N4-hydroxypseudoisocytidine, N4-hydroxy-5-methylcytidine, N4-hydroxy-5-aminocytidine, N4-hydroxy-5-hydroxycytidine, N4-hydroxy-5-methyl-5-azacytidine, N4-hydroxy-5-amino-5-azacytidine, N4-hydroxy-5-hydroxy-5-azacytidine, N4-hydroxy-5-methylpseudoisocytidine, N4-hydroxy-5-aminopseudoisocytidine, N4-hydroxy-5-hydroxypseudoisocytidine, 2-thio-N4-methyl-5-methylcytidine, 2-thio-N4-methyl-5-aminocytidine, 2-thio-N4-methyl-5-hydroxycytidine, 2-thio-N4-methyl-5-methyl-5-azacytidine, 2-thio-N4-methyl-5-amino-5-azacytidine, 2-thio-N4-methyl-5-hydroxy-5-azacytidine, 2-thio-N4-methyl-5-methylpseudoisocytidine, 2-thio-N4-methyl-5-aminopseudoisocytidine, 2-thio-N4-methyl-5-hydroxypseudoisocytidine, 2-thio-N4-amino-5-azacytidine, 2-thio-N4-aminopseudoisocytidine, 2-thio-N4-amino-5-methylcytidine, 2-thio-N4-amino-5-aminocytidine, 2-thio-N4-amino-5-hydroxycytidine, 2-thio-N4-amino-5-methyl-5-azacytidine, 2-thio-N4-amino-5-amino-5-azacytidine, 2-thio-N4-amino-5-hydroxy-5-azacytidine, 2-thio-N4-amino-5-methylpseudoisocytidine, 2-thio-N4-amino-5-aminopseudoisocytidine, 2-thio-N4-amino-5-hydroxypseudoisocytidine, 2-thio-N4-hydroxy-5-azacytidine, 2-thio-N4-hydroxypseudoisocytidine, 2-thio-N4-hydroxy-5-methylcytidine, N4-hydroxy-5-aminocytidine, 2-thio-N4-hydroxy-5-hydroxycytidine, 2-thio-N4-hydroxy-5-methyl-5-azacytidine, 2-thio-N4-hydroxy-5-amino-5-azacytidine, 2-thio-N4-hydroxy-5-hydroxy-5-azacytidine, 2-thio-N4-hydroxy-5-methylpseudoisocytidine, 2-thio-N4-hydroxy-5-aminopseudoisocytidine, 2-thio-N4-hydroxy-5-hydroxypseudoisocytidine, N6-methyladenosine, N6-aminoadenosine, N6-hydroxyadenosine, 7-deazaadenosine, 8-azaadenosine, N6-methyl-7-deazaadenosine, N6-methyl-8-azaadenosine, 7-deaza-8-azaadenosine, N6-methyl-7-deaza-8-azaadenosine, N6-amino-7-deazaadenosine, N6-amino-8-azaadenosine, N6-amino-7-deaza-8-azaadenosine, N6-hydroxyadenosine, N6-hydroxy-7-deazaadenosine, N6-hydroxy-8-azaadenosine, N6-hydroxy-7-deaza-8-azaadenosine, 6-thioguanosine, 7-deazaguanosine, 8-azaguanosine, 6-thio-7-deazaguanosine, 6-thio-8-azaguanosine, 7-deaza-8-azaguanosine, and 6-thio-7-deaza-8-azaguanosine.

In some embodiments, the one or more non-canonical nucleotides are selected from 5-hydroxycytidine, 5-methylcytidine, 5-hydroxymethylcytidine, 5-carboxycytidine, 5-formylcytidine, 5-methoxycytidine, 5-hydroxyuridine, 5-hydroxymethyluridine, 5-carboxyuridine, 5-formyluridine, 5-methoxyuridine, pseudouridine, 5-hydroxypseudouridine, 5-methylpseudouridine, 5-hydroxymethylpseudouridine, 5-carboxypseudouridine, 5-formylpseudouridine, and 5-methoxypseudouridine. In some embodiments, at least 50%, or at least 55%, or at least 60%, or at least 65%, or at least 70%, or at least 75%, or at least 80%, or at least 85%, or at least 90%, or at least 95%, or 100% of the non-canonical nucleotides are one or more of 5-hydroxycytidine, 5-methylcytidine, 5-hydroxymethylcytidine, 5-carboxycytidine, 5-formylcytidine, 5-methoxycytidine, 5-hydroxyuridine, 5-methyluridine, 5-hydroxymethyluridine, 5-carboxyuridine, 5-formyluridine, 5-methoxyuridine, pseudouridine, 5-hydroxypseudouridine, 5-methylpseudouridine, 5-hydroxymethylpseudouridine, 5-carboxypseudouridine, 5-formylpseudouridine, and 5-methoxypseudouridine.

In some embodiments, at least about 50%, or at least about 55%%, or at least 60%, or at least 65%, or at least 70%, or at least 75%, or at least 80%, or at least 85%, or at least 90%, or at least 95%, or 100% of cytidine residues are non-canonical nucleotides selected from 5-hydroxycytidine, 5-methylcytidine, 5-hydroxymethylcytidine, 5-carboxycytidine, 5-formylcytidine, 5-methoxycytidine.

In some embodiments, at least about 20%, or about 30%, or about 40%, or about 50%, or at least about 55%, or at least 60%, or at least 65%, or at least 70%, or at least 75%, or at least 80%, or at least 85%, or at least 90%, or at least 95%, or 100% of uridine residues are non-canonical nucleotides selected from 5-hydroxyuridine, 5-methyluridine, 5-hydroxymethyluridine, 5-carboxyuridine, 5-formyluridine, 5-methoxyuridine, pseudouridine, 5-hydroxypseudouridine, 5-methylpseudouridine, 5-hydroxymethylpseudouridine, 5-carboxypseudouridine, 5-formylpseudouridine, and 5-methoxypseudouridine.

In some embodiments, at least about 10% (e.g. 10%, or about 20%, or about 30%, or about 40%, or about 50%) of guanosine residues are non-canonical nucleotides, and the non-canonical nucleotide is optionally 7-deazaguanosine. In some embodiments, the RNA contains no more than about 50% 7-deazaguanosine in place of guanosine residues.

In some embodiments, the synthetic RNA molecule does not contain non-canonical nucleotides in place of adenosine residues.

Note that alternative naming schemes exist for certain non-canonical nucleotides. For example, in certain situations, 5-methylpseudouridine can be referred to as “3-methylpseudouridine” or “N3-methylpseudouridine” or “1-methylpseudouridine” or “N1-methylpseudouridine”. Nucleotides that contain the prefix “amino” can refer to any nucleotide that contains a nitrogen atom bound to the atom at the stated position of the nucleotide, for example, 5-aminocytidine can refer to 5-aminocytidine, 5-methylaminocytidine, and 5-nitrocytidine. Similarly, nucleotides that contain the prefix “methyl” can refer to any nucleotide that contains a carbon atom bound to the atom at the stated position of the nucleotide, for example, 5-methylcytidine can refer to 5-methylcytidine, 5-ethylcytidine, and 5-hydroxymethylcytidine, nucleotides that contain the prefix “thio” can refer to any nucleotide that contains a sulfur atom bound to the atom at the given position of the nucleotide, and nucleotides that contain the prefix “hydroxy” can refer to any nucleotide that contains an oxygen atom bound to the atom at the given position of the nucleotide, for example, 5-hydroxyuridine can refer to 5-hydroxyuridine and uridine with a methyl group bound to an oxygen atom, wherein the oxygen atom is bound to the atom at the 5C position of the uridine.

Certain non-canonical nucleotides can be incorporated more efficiently than other non-canonical nucleotides into RNA molecules by RNA polymerases that are commonly used for in vitro transcription, due in part to the tendency of these certain non-canonical nucleotides to participate in standard base-pairing interactions and base-stacking interactions, and to interact with the RNA polymerase in a manner similar to that in which the corresponding canonical nucleotide interacts with the RNA polymerase. As a result, certain nucleotide mixtures containing one or more non-canonical nucleotides can be beneficial in part because in vitro-transcription reactions containing these nucleotide mixtures can yield a large quantity of RNA. Certain embodiments are therefore directed to a nucleotide mixture containing one or more nucleotides that includes one or more substitutions at the 2C and/or 4C and/or 5C positions in the case of a pyrimidine or the 6C and/or 7N and/or 8C positions in the case of a purine. Nucleotide mixtures include, but are not limited to (numbers preceding each nucleotide indicate an exemplary fraction of the non-canonical nucleotide triphosphate in an in vitro-transcription reaction, for example, 0.2 pseudoisocytidine refers to a reaction containing adenosine-5′-triphosphate, guanosine-5′-triphosphate, uridine-5′-triphosphate, cytidine-5′-triphosphate, and pseudoisocytidine-5′-triphosphate, wherein pseudoisocytidine-5′-triphosphate is present in the reaction at an amount approximately equal to 0.2 times the total amount of pseudoisocytidine-5′-triphosphate+cytidine-5′-triphosphate that is present in the reaction, with amounts measured either on a molar or mass basis, and wherein more than one number preceding a nucleoside indicates a range of exemplary fractions): 1.0 pseudouridine, 0.1-0.8 2-thiouridine, 0.1-0.8 5-methyluridine, 0.2-1.0 5-hydroxyuridine, 0.2-1.0 5-methoxyuridine, 0.1-1.0 5-aminouridine, 0.1-1.0 4-thiouridine, 0.1-1.0 2-thiopseudouridine, 0.1-1.0 4-thiopseudouridine, 0.1-1.0 5-hydroxypseudouridine, 0.2-1 5-methylpseudouridine, 0.2-1.0 5-methoxypseudouridine, 0.1-1.0 5-aminopseudouridine, 0.2-1.0 2-thiocytidine, 0.1-0.8 pseudoisocytidine, 0.2-1.0 5-methylcytidine, 0.2-1.0 5-hydroxycytidine, 0.2-1.0 5-hydroxymethylcytidine, 0.2-1.0 5-methoxycytidine, 0.1-1.0 5-aminocytidine, 0.2-1.0 N4-methylcytidine, 0.2-1.0 5-methylpseudoisocytidine, 0.2-1.0 5-hydroxypseudoisocytidine, 0.2-1.0 5-aminopseudoisocytidine, 0.2-1.0 N4-methylpseudoisocytidine, 0.2-1.0 2-thiopseudoisocytidine, 0.2-1.0 7-deazaguanosine, 0.2-1.0 6-thioguanosine, 0.2-1.0 6-thio-7-deazaguanosine, 0.2-1.0 8-azaguanosine, 0.2-1.0 7-deaza-8-azaguanosine, 0.2-1.0 6-thio-8-azaguanosine, 0.1-0.5 7-deazaadenosine, and 0.1-0.5 N6-methyladenosine.

In some embodiments, the RNA comprising one or more non-canonical nucleotides composition or synthetic polynucleotide composition (e.g., which may be prepared by in vitro transcription) contains substantially or entirely the canonical nucleotide at positions having adenine or “A” in the genetic code. The term “substantially” in this context refers to at least 90%. In these embodiments, the RNA composition or synthetic polynucleotide composition may further contain (e.g., consist of) 7-deazaguanosine at positions with “G” in the genetic code as well as the corresponding canonical nucleotide “G”, and the canonical and non-canonical nucleotide at positions with G may be in the range of 5:1 to 1:5, or in some embodiments in the range of 2:1 to 1:2. In these embodiments, the RNA composition or synthetic polynucleotide composition may further contain (e.g., consist of) one or more (e.g., two, three or four) of 5-hydroxycytidine, 5-methylcytidine, 5-hydroxymethylcytidine, 5-carboxycytidine, 5-formylcytidine, 5-methoxycytidine at positions with “C” in the genetic code as well as the canonical nucleotide “C”, and the canonical and non-canonical nucleotide at positions with C may be in the range of 5:1 to 1:5, or in some embodiments in the range of 2:1 to 1:2. In some embodiments, the level of non-canonical nucleotide at positions of “C” are as described in the preceding paragraph. In these embodiments, the RNA composition or synthetic polynucleotide composition may further contain (e.g., consist of) one or more (e.g., two, three, or four) of 5-hydroxyuridine, 5-methyluridine, 5-hydroxymethyluridine, 5-carboxyuridine, 5-formyluridine, 5-methoxyuridine, pseudouridine, 5-hydroxypseudouridine, 5-methylpseudouridine, 5-hydroxymethylpseudouridine, 5-carboxypseudouridine, 5-formylpseudouridine, and 5-methoxypseudouridine at positions with “U” in the genetic code as well as the canonical nucleotide “U”, and the canonical and non-canonical nucleotide at positions with “U” may be in the range of 5:1 to 1:5, or in some embodiments in the range of 2:1 to 1:2. In some embodiments, the level of non-canonical nucleotide at positions of “U” are as described in the preceding paragraph.

In embodiments, combining certain non-canonical nucleotides can be beneficial in part because the contribution of non-canonical nucleotides to lowering the toxicity of RNA molecules can be additive. Certain embodiments are therefore directed to a nucleotide mixture, wherein the nucleotide mixture contains more than one of the non-canonical nucleotides listed above, for example, the nucleotide mixture contains both pseudoisocytidine and 7-deazaguanosine or the nucleotide mixture contains both N4-methylcytidine and 7-deazaguanosine, etc. In one embodiment, the nucleotide mixture contains more than one of the non-canonical nucleotides listed above, and each of the non-canonical nucleotides is present in the mixture at the fraction listed above, for example, the nucleotide mixture contains 0.1-0.8 pseudoisocytidine and 0.2-1.0 7-deazaguanosine or the nucleotide mixture contains 0.2-1.0 N4-methylcytidine and 0.2-1.0 7-deazaguanosine, etc.

In certain situations, for example, when it may not be necessary or desirable to maximize the yield of an in vitro-transcription reaction, nucleotide fractions other than those given above may be used. The exemplary fractions and ranges of fractions listed above relate to nucleotide-triphosphate solutions of typical purity (greater than 90% purity). Larger fractions of these and other nucleotides can be used by using nucleotide-triphosphate solutions of greater purity, for example, greater than about 95% purity or greater than about 98% purity or greater than about 99% purity or greater than about 99.5% purity, which can be achieved, for example, by purifying the nucleotide triphosphate solution using existing chemical-purification technologies such as high-pressure liquid chromatography (HPLC) or by other means. In one embodiment, nucleotides with multiple isomers are purified to enrich the desired isomer.

In some embodiments, the one or more non-canonical nucleotides avoids substantial cellular toxicity.

In some embodiments, the non-canonical nucleotides comprise one or more of 5-hydroxycytidine, 5-methylcytidine, 5-hydroxymethylcytidine, 5-carboxycytidine, 5-formylcytidine, 5-methoxycytidine, pseudouridine, 5-hydroxyuridine, 5-methyluridine, 5-hydroxymethyluridine, 5-carboxyuridine, 5-formyluridine, 5-methoxyuridine, 5-hydroxypseudouridine, 5-methylpseudouridine, 5-hydroxymethylpseudouridine, 5-carboxypseudouridine, 5-formylpseudouridine, and 5-methoxypseudouridine, optionally at an amount of at least 50%, or at least 60%, or at least 70%, or at least 80%, or at least 90%, or 100% of the non-canonical nucleotides.

In some embodiments, at least about 50% of cytidine residues are non-canonical nucleotides, and which are selected from 5-hydroxycytidine, 5-methylcytidine, 5-hydroxymethylcytidine, 5-carboxycytidine, 5-formylcytidine, and 5-methoxycytidine.

In some embodiments, at least about 75% or at least about 90% of cytidine residues are non-canonical nucleotides, and the non-canonical nucleotides are selected from 5-hydroxycytidine, 5-methylcytidine, 5-hydroxymethylcytidine, 5-carboxycytidine, 5-formylcytidine, and 5-methoxycytidine.

In some embodiments, at least about 20% of uridine, or at least about 40%, or at least about 50%, or at least about 75%, or at about least 90% of uridine residues are non-canonical nucleotides, and the non-canonical are selected from pseudouridine, 5-hydroxyuridine, 5-methyluridine, 5-hydroxymethyluridine, 5-carboxyuridine, 5-formyluridine, 5-methoxyuridine, 5-hydroxypseudouridine, 5-methylpseudouridine, 5-hydroxymethylpseudouridine, 5-carboxypseudouridine, 5-formylpseudouridine, and 5-methoxypseudouridine.

In some embodiments, at least about 40%, or at least about 50%, or at least about 75%, or at about least 90% of uridine residues are non-canonical nucleotides, and the non-canonical nucleotides are selected from pseudouridine, 5-hydroxyuridine, 5-methyluridine, 5-hydroxymethyluridine, 5-carboxyuridine, 5-formyluridine, 5-methoxyuridine, 5-hydroxypseudouridine, 5-methylpseudouridine, 5-hydroxymethylpseudouridine, 5-carboxypseudouridine, 5-formylpseudouridine, and 5-methoxypseudouridine.

In some embodiments, at least about 10% of guanine residues are non-canonical nucleotides, and the non-canonical nucleotide is optionally 7-deazaguanosine. In some embodiments, the synthetic RNA comprises no more than about 50% 7-deazaguanosine in place of guanosine residues. In some embodiments, the synthetic RNA does not comprise non-canonical nucleotides in place of adenosine residues.

In some embodiments, the synthetic RNA comprises a 5′ cap structure. In some embodiments, the synthetic RNA comprises a Kozak consensus sequence. In some embodiments, the synthetic RNA comprises a 5′-UTR which comprises a sequence that increases RNA stability in vivo, and the 5′-UTR optionally comprises an alpha-globin or beta-globin 5′-UTR. In some embodiments, the synthetic RNA comprises a 3′-UTR which comprises a sequence that increases RNA stability in vivo, and the 3′-UTR optionally comprises an alpha-globin or beta-globin 3′-UTR. In some embodiments, the synthetic RNA comprises a 5′-UTR which comprises a microRNA binding site that modulates RNA stability in a cell type-specific manner. In some embodiments, the synthetic RNA comprises a 3′-UTR which comprises a microRNA binding site that modulates RNA stability in a cell type-specific manner. In some embodiments, the synthetic RNA comprises a 3′ poly(A) tail. In some embodiments, the synthetic RNA comprises a 3′ poly(A) tail which comprises from about 20 nucleotides to about 250 nucleotides.

In some embodiments, the synthetic RNA comprises about 200 nucleotides to about 5000 nucleotides. In some embodiments, the synthetic RNA comprises from about 500 to about 2000 nucleotides, or about 500 to about 1500 nucleotides, or about 500 to about 1000 nucleotides.

### Methods of Treatment

In various embodiments, the present methods and compositions find use in methods of treating, preventing, or ameliorating a disease, disorder, and/or condition. For instance, in some embodiments, the described methods of in vivo delivery, including administration strategies, and formulations are used in a method of treatment. In some methods, the described methods reduce symptoms associated with a disease. In some embodiments, the methods eliminate the underlying cause of the disease. In some embodiments, the methods are used in the treatment of a disease requiring immunosuppression. In some embodiments, the methods reduce inflammation. In some embodiments, the methods reduce immune response.

In embodiments, the disclosed composition is suitable for use in the treatment of amyotrophic lateral sclerosis (ALS), spinal cord injury, degenerative disc disease, coronary artery disease, acute myocardial infarction, alcoholic liver cirrhosis, hepatitis C virus (HCV)-induced cirrhosis, multiple sclerosis (MS), osteoarthritis (OA), osteoarthritis of the knee, kidney allograft, critical limb ischemia, ischemic cardiomyopathy, Crohn's disease, idiopathic pulmonary fibrosis, anal fistula, spinal cord injury, systemic lupus erythematosus (SLE), acute respiratory distress syndrome (ARDS), acute graft-versus-host disease (aGvHD), preterm bronchopulmonary dysplasia (BPD), autism nonischemic heart failure, and/or Type 2 diabetes mellitus.

In embodiments, the present methods relate to therapeutic use in autoimmune diseases or disorders.

Examples of autoimmune diseases or disorders that may be treated or prevented by the present invention include, but are not limited to, alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre syndrome, Hashimoto's thyroiditis, idiopathic pulmonary fibrosis, idiopathic thrombocytopenia purpura (ITP), irritable bowel disease (IBD), IgA neuropathy, juvenile arthritis, lichen planus, lupus erthematosus, Meniere's disease, mixed connective tissue disease, multiple sclerosis, type 1 or immune-mediated diabetes mellitus, myasthenia gravis, pemphigus vulgaris, pernicious anemia, polyarteritis nodosa, polychrondritis, polyglandular syndromes, polymyalgia rheumatica, polymyositis and dermatomyositis, primary agammaglobulinemia, primary biliary cirrhosis, psoriasis, psoriatic arthritis, Raynauld's phenomenon, Reiter's syndrome, Rheumatoid arthritis, sarcoidosis, scleroderma, stiff-man syndrome, systemic lupus erythematosus, lupus erythematosus, takayasu arteritis, temporal arteristis, giant cell arteritis, ulcerative colitis, uveitis, vitiligo and Wegener's granulomatosis. Preferably autoimmune disorders that may be treated or prevented by the present compositions include rheumatoid arthritis, type 1 diabetes mellitus, multiple sclerosis, systemic lupus erythematosus, and atopy.

In embodiments, the present methods relate to therapeutic use in degenerative diseases or disorders.

A degenerative diseases or disorders is a disease in which the function or structure of the affected tissues or organs will progressively deteriorate over time. Examples of degenerative diseases that can be treated or prevented with the present invention include Amyotrophic Lateral Sclerosis (ALS), Alzheimer's disease, Parkinson's Disease, Multiple system atrophy, Niemann Pick disease, Atherosclerosis, Progressive supranuclear palsy, Tay-Sachs Disease, Diabetes, Heart Disease, Keratoconus, Inflammatory Bowel Disease (IBD), Prostatitis, Osteoarthritis, Osteoporosis, Rheumatoid Arthritis, Huntington's Disease, Chronic traumatic encephalopathy, Epilepsy, Dementia, Renal failure, Multiple sclerosis, Malaria with CNS degeneration, Neuro-AIDS, Lysosomal storage diseases, Encephalatis of viral, bacterial or autoimmune origin.

In embodiments, the present methods relate to therapeutic use in a lung diseases or disorders.

In embodiments, the lung disease or disorder is a lung disease or disorder that would benefit therapeutically from suppression of immune responses in the lung. In some embodiments, inflammation is associated with the lung disease or disorder.

In some embodiments, the lung disease or disorder is selected from Asbestosis, Asthma, Bronchiectasis, Bronchitis, Chronic Cough, Chronic Obstructive Pulmonary Disease (COPD), Common Cold, Croup, Cystic Fibrosis, Hantavirus, Idiopathic Pulmonary Fibrosis, Influenza, Lung Cancer, Pandemic Flu, Pertussis, Pleurisy, Pneumonia, Pulmonary Embolism, Pulmonary Hypertension, Respiratory Syncytial Virus (RSV), Sarcoidosis, Sleep Apnea, Spirometry, Sudden Infant Death Syndrome (SIDS), and Tuberculosis.

In some embodiments, the lung disease or disorder is chronic obstructive pulmonary disease (COPD), reactive airway disease such as asthma, bronchiolitis, acute lung injury, lung allograft rejection (acute or chronic), pulmonary fibrosis, interstitial lung disease or hypersensitivity pneumonitis. In embodiments, the disease or disorder is an acute lung injury (ALI). In embodiments, the ALI is a pulmonary disorder that can be induced directly by inhalation of chemicals (chemical induced acute lung injury) or other means (e.g. infection) or can be induced indirectly by systemic injury (e.g. infection). Acute lung injury includes subcategories of respiratory distress syndromes including infant respiratory distress syndrome (IRDS), hyaline membrane disease (HMD), neonatal respiratory distress syndrome (NRDS), respiratory distress syndrome of newborn (RDSN), surfactant deficiency disorder (SDD), acute respiratory distress syndrome (ARDS), respiratory complication from systemic inflammatory response syndrome (SIRS), or severe acute respiratory syndrome (SARS).

In embodiments, the present invention relates to the therapeutic use of the present MSCs for the treatment of one or more symptoms associated with a viral infection.

In embodiments, the composition is suitable for use in the treatment of an infectious disease, optionally selected from an infection with a pathogen, optionally selected from a bacterium, virus, fungus, or parasite.

In embodiments, the pathogen is a virus. In embodiments, the virus is: (a) an influenza virus, optionally selected from Type A, Type B, Type C, and Type D influenza viruses, or (b) a member of the Coronaviridae family, optionally selected from (i) a betacoronavirus, optionally selected from Severe acute respiratory syndrome coronavirus 2 (SARS-CoV-2), SARS-CoV, Middle East Respiratory Syndrome-Corona Virus (MERS-CoV), HCoV-HKU1, and HCoV-OC43 or (ii) an alphacoronavirus, optionally selected from HCoV-NL63 and HCoV-229E.

In embodiments, the virus is SARS-CoV-2. In embodiments, the virus is SARS-CoV-2, which has caused COVID-19. In embodiments, the COVID-19 is characterized by one or more of fever, cough, shortness of breath, diarrhea, upper respiratory symptoms, lower respiratory symptoms, pneumonia, and respiratory distress.

In some embodiments, the composition is suitable for use in the treatment of an infection, wherein the infection is a coronavirus infection. In some embodiments, the coronavirus infection is one or more of Severe acute respiratory syndrome coronavirus 2 (SARS-CoV-2), SARS-CoV, Middle East Respiratory Syndrome-Corona Virus (MERS-CoV), HCoV-HKU1, HCoV-OC43, HCoV-NL63, and HCoV-229E. In various embodiments, the coronavirus infection is SARS or COVID-19. In further embodiments, the subject is infected by SARS-CoV-2.

In embodiments, the therapy prevents or mitigates development of acute respiratory distress syndrome (ARDS) in a patient when administered. In embodiments, the therapy improves oxygenation in a patient when administered. In embodiments, the therapy improves systemic blood pressure oxygenation in a patient when administered, e.g. reducing or mitigating shock, e.g. requiring less pressor support. In embodiments, the therapy improves lung and/or alveolar permeability in a patient when administered.

In embodiments, the therapy prevents or mitigates a transition from respiratory distress to cytokine imbalance in a patient when administered. In embodiments, the therapy reverses or prevents a cytokine storm in a patient when administered. In embodiments, the therapy reverses or prevents a cytokine storm in the lungs or systemically in a patient when administered. In embodiments, the cytokine storm is selected from one or more of systemic inflammatory response syndrome, cytokine release syndrome, macrophage activation syndrome, and hemophagocytic lymphohistiocytosis.

In embodiments, the therapy reverses or prevents excessive production of one or more inflammatory cytokines in a patient when administered. In embodiments, the inflammatory cytokine is one or more of IL-6, IL-1, IL-1 receptor antagonist (IL-1ra), IL-2ra, IL-10, IL-18, TNFα, interferon-γ, CXCL10, and CCL7.

In embodiments, the present invention relates to the therapeutic use of the present MSCs for the treatment of one or more symptoms associated with a coronavirus infection.

Coronaviruses (CoVs) are members of the family Coronaviridae, including betacoronavirus and alphacoronavirus—respiratory pathogens that have relatively recently become known to invade humans. The Coronaviridae family includes such betacoronavirus as Severe acute respiratory syndrome coronavirus 2 (SARS-CoV-2), SARS-CoV, Middle East Respiratory Syndrome-Corona Virus (MERS-CoV), HCoV-HKU1, and HCoV-OC43. Alphacoronavirus includes, e.g., HCoV-NL63 and HCoV-229E. In embodiments, the present invention relates to the therapeutic use of the present MSCs for the treatment of one or more symptoms of infection with any of Severe acute respiratory syndrome coronavirus 2 (SARS-CoV-2), SARS-CoV, Middle East Respiratory Syndrome-Corona Virus (MERS-CoV), HCoV-HKU1, and HCoV-OC43. Alphacoronavirus includes, e.g., HCoV-NL63 and HCoV-229E.

Without wishing to be bound by theory, coronaviruses invade cells through utilization of their “spike” surface glycoprotein that is responsible for viral recognition of Angiotensin Converting Enzyme 2 (ACE2), a transmembrane receptor on mammalian hosts that facilitate viral entrance into host cells. (Zhou et al., A pneumonia outbreak associated with a new coronavirus of probable bat origin, Nature 2020).

Symptoms associated with coronavirus infections include, but are not limited to, fever, tiredness, dry cough, aches and pains, shortness of breath and other breathing difficulties, diarrhea, upper respiratory symptoms (e.g. sneezing, runny nose, nasal congestion, cough, sore throat), and/or pneumonia. In embodiments, the present compositions and methods are useful in treating or mitigating any of these symptoms.

In embodiments, the present invention relates to the therapeutic use of the present MSCs for the treatment of one or more symptoms of infection with SARS-CoV-2, including Coronavirus infection 2019 (COVID-19), caused by SARS-CoV-2 (e.g., 2019-nCoV).

In some settings, including subjects afflicted with coronavirus infections, it is possible that the morbidity and mortality of pulmonary viral infection is related to an exaggerated or overwhelming inflammatory response. In varying clinical circumstances this response can be described as “cytokine response syndrome”, “cytokine storm”, or “secondary hemophagocytic lymphohistiocytosis” (sHLH). In embodiments, the present compositions and methods are useful in treating or mitigating any of these exaggerated or overwhelming inflammatory responses. Collectively it is surmised that these highly proinflammatory states can lead to death due to pulmonary collapse such as acute respiratory distress syndrome (ARDS) or systemic, multi-organ failure affecting organs such as liver, kidney, heart and brain. In embodiments, the present MSCs treat or mitigate a “cytokine response syndrome”, “cytokine storm”, or “secondary hemophagocytic lymphohistiocytosis” (sHLH).

In embodiments, COVID-19 is characterized, in part, by elevation of Interleukin-2 (IL-2), Interleukin-7 (IL-7), granulocyte colony stimulating factor (GCSF), interferon-gamma inducible protein 10, monocyte chemoattractant protein-1 (MCP-1), macrophage inflammatory protein 1-alpha (MIP1a), and tumor necrosis factor-alpha (TNFα). In embodiments, the present compositions and methods are useful in treating or mitigating increases of any of these factors.

In embodiments, the present MSCs prevent a COVID-19 patient from having a disease that develops from respiratory distress to cytokine storm.

In embodiments, the present MSCs treat or mitigate ARDS.

In some embodiments, a cytokine storm is associated with COVID-19 and is treated or mitigated via a method comprising administering to a subject in need thereof an effective amount of MSCs effective for the treatment of a coronavirus infection and/or a cytokine storm associated with a coronavirus infection, wherein the subject has abnormal (e.g. increased or decreased) expression or activity of one or more of IL-6, IL-1, TNF, interferon-γ, CXCL10, CCL7, IL-1 receptor antagonist (IL-1ra), IL-2ra, IL-10, IL-18, CCL2/MCP-1, CCL5/RANTES, CCL7/MCP-3, MCP-2, tumour necrosis factor-alpha (TNFα), interferon-γ (IFNg), CXCL10, CXC3, Granulocyte colony stimulatory factor (GCSF), Macrophage inflammatory protein 1 alpha (MIP-1a), IL-22, and Interferon gamma induced protein 10 (IP-10).

In some embodiments, the subject has a modulated (e.g. decreased or increased) expression or activity of one or more of IL-6, IL-1, TNF, interferon-γ, CXCL10, CCL7, IL-1 receptor antagonist (IL-1ra), IL-2ra, IL-10, IL-18, CCL2/MCP-1, CCL5/RANTES, CCL7/MCP-3, MCP-2, tumour necrosis factor-alpha (TNFα), interferon-γ (IFNg), CXCL10, CXC3, Granulocyte colony stimulatory factor (GCSF), Macrophage inflammatory protein 1 alpha (MIP-1a), IL-22, and Interferon gamma induced protein 10 (IP-10).

In an aspect there is provided a method of treating cancer, comprising: (a) obtaining a mesenchymal stem cell (MSC), the MSC having been obtained from reprogramming an induced pluripotent stem cell (iPSC), the reprogramming comprising contacting the iPSC with one or more synthetic RNA molecules encoding a reprogramming factor and having a protein secretion signature, the protein secretion signature comprising an increased secretion of one or more proteins selected from MIP-1 alpha, SDF-1 alpha, IL-27, LIF, IL-1 beta, IL-2, IL-5, IL-12p70, IL-13, IL-17A, IL-31, G-CSF/CSF-3, IFN-gamma, TNF-alpha, HGF, MCP-1, IL-9, bNGF, MIP-3 alpha, Gro-alpha/KC, IL-1alpha, IL-23, MMP-1, IL-18, M-CSF, IL-21, M-CSF, IL-21, CD40L, IL-22, VEGF-A, BLC, Tweak, ENA-78 (LIX), MCP-3, MIF, and Eotaxin-3 and/or a decreased secretion of one or more proteins selected from IL-6, IL-8, and IL-4, wherein the increased and/or decreased secretion is relative to a bone marrow-derived MSC; and (b) administering an effective amount of the MSC substantially having the protein secretion signature for therapy to a patient in need thereof.

In embodiments, the MSC is further engineered to express and/or secrete a soluble protein. In embodiments, the soluble protein is a TNF family receptor ligand, optionally selected from TRAIL/TNFSF10 and TNFα. In embodiments, the soluble protein is an interleukin, optionally selected from IL-2, IL-6, IL-7, IL-12, IL-15, IL-18, and IL-21, inclusive or a chimeric protein thereof (e.g. linked with a glycine/serine linker or self-cleaving peptide). In embodiments, the soluble protein is Flt-3 ligand.

In embodiments, the cancer is one or more of basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema, and Meigs' syndrome

In embodiments, the iPSC is derived from a human. In embodiments, the iPSC is derived from a subject who is not intended to receive the therapy. In embodiments, the iPSC is allogeneic to the patient intended to receive the therapy. In embodiments, the iPSC is from a master cell bank. In embodiments, the MSC is characterized by low or reduced inflammation. In embodiments, the MSC is characterized by low or reduced immunogenicity. In embodiments, the MSC is self-renewing. In embodiments, the MSC is multipotent. In embodiments, the MSC is immune inhibitory. In embodiments, the MSC is suitable for in vitro expansion without substantial loss of immunosuppressive properties. In embodiments, the MSC substantially expresses one or more of CD73, CD90, and CD105. In embodiments, the MSC substantially does not express one or more of CD14, CD34 and CD45.

In embodiments, the synthetic RNA molecule is mRNA. In embodiments, the synthetic RNA molecule is mRNA comprising one or more non-canonical nucleotides selected from 2-thiouridine, 5-azauridine, pseudouridine, 4-thiouridine, 5-methyluridine, 5-methylpseudouridine, 5-aminouridine, 5-aminopseudouridine, 5-hydroxyuridine, 5-hydroxypseudouridine, 5-methoxyuridine, 5-methoxypseudouridine, 5-ethoxyuridine, 5-ethoxypseudouridine, 5-hydroxymethyluridine, 5-hydroxymethylpseudouridine, 5-carboxyuridine, 5-carboxypseudouridine, 5-formyluridine, 5-formylpseudouridine, 5-methyl-5-azauridine, 5-amino-5-azauridine, 5-hydroxy-5-azauridine, 5-methylpseudouridine, 5-aminopseudouridine, 5-hydroxypseudouridine, 4-thio-5-azauridine, 4-thiopseudouridine, 4-thio-5-methyluridine, 4-thio-5-aminouridine, 4-thio-5-hydroxyuridine, 4-thio-5-methyl-5-azauridine, 4-thio-5-amino-5-azauridine, 4-thio-5-hydroxy-5-azauridine, 4-thio-5-methylpseudouridine, 4-thio-5-aminopseudouridine, 4-thio-5-hydroxypseudouridine, 2-thiocytidine, 5-azacytidine, pseudoisocytidine, N4-methylcytidine, N4-aminocytidine, N4-hydroxycytidine, 5-methylcytidine, 5-aminocytidine, 5-hydroxycytidine, 5-methoxycytidine, 5-ethoxycytidine, 5-hydroxymethylcytidine, 5-carboxycytidine, 5-formylcytydine, 5-methyl-5-azacytidine, 5-amino-5-azacytidine, 5-hydroxy-5-azacytidine, 5-methylpseudoisocytidine, 5-aminopseudoisocytidine, 5-hydroxypseudoisocytidine, N4-methyl-5-azacytidine, N4-methylpseudoisocytidine, 2-thio-5-azacytidine, 2-thiopseudoisocytidine, 2-thio-N4-methylcytidine, 2-thio-N4-aminocytidine, 2-thio-N4-hydroxycytidine, 2-thio-5-methylcytidine, 2-thio-5-aminocytidine, 2-thio-5-hydroxycytidine, 2-thio-5-methyl-5-azacytidine, 2-thio-5-amino-5-azacytidine, 2-thio-5-hydroxy-5-azacytidine, 2-thio-5-methylpseudoisocytidine, 2-thio-5-aminopseudoisocytidine, 2-thio-5-hydroxypseudoisocytidine, 2-thio-N4-methyl-5-azacytidine, 2-thio-N4-methylpseudoisocytidine, N4-methyl-5-methylcytidine, N4-methyl-5-aminocytidine, N4-methyl-5-hydroxycytidine, N4-methyl-5-methyl-5-azacytidine, N4-methyl-5-amino-5-azacytidine, N4-methyl-5-hydroxy-5-azacytidine, N4-methyl-5-methylpseudoisocytidine, N4-methyl-5-aminopseudoisocytidine, N4-methyl-5-hydroxypseudoisocytidine, N4-amino-5-azacytidine, N4-aminopseudoisocytidine, N4-amino-5-methylcytidine, N4-amino-5-aminocytidine, N4-amino-5-hydroxycytidine, N4-amino-5-methyl-5-azacytidine, N4-amino-5-amino-5-azacytidine, N4-amino-5-hydroxy-5-azacytidine, N4-amino-5-methylpseudoisocytidine, N4-amino-5-aminopseudoisocytidine, N4-amino-5-hydroxypseudoisocytidine, N4-hydroxy-5-azacytidine, N4-hydroxypseudoisocytidine, N4-hydroxy-5-methylcytidine, N4-hydroxy-5-aminocytidine, N4-hydroxy-5-hydroxycytidine, N4-hydroxy-5-methyl-5-azacytidine, N4-hydroxy-5-amino-5-azacytidine, N4-hydroxy-5-hydroxy-5-azacytidine, N4-hydroxy-5-methylpseudoisocytidine, N4-hydroxy-5-aminopseudoisocytidine, N4-hydroxy-5-hydroxypseudoisocytidine, 2-thio-N4-methyl-5-methylcytidine, 2-thio-N4-methyl-5-aminocytidine, 2-thio-N4-methyl-5-hydroxycytidine, 2-thio-N4-methyl-5-methyl-5-azacytidine, 2-thio-N4-methyl-5-amino-5-azacytidine, 2-thio-N4-methyl-5-hydroxy-5-azacytidine, 2-thio-N4-methyl-5-methylpseudoisocytidine, 2-thio-N4-methyl-5-aminopseudoisocytidine, 2-thio-N4-methyl-5-hydroxypseudoisocytidine, 2-thio-N4-amino-5-azacytidine, 2-thio-N4-aminopseudoisocytidine, 2-thio-N4-amino-5-methylcytidine, 2-thio-N4-amino-5-aminocytidine, 2-thio-N4-amino-5-hydroxycytidine, 2-thio-N4-amino-5-methyl-5-azacytidine, 2-thio-N4-amino-5-amino-5-azacytidine, 2-thio-N4-amino-5-hydroxy-5-azacytidine, 2-thio-N4-amino-5-methylpseudoisocytidine, 2-thio-N4-amino-5-aminopseudoisocytidine, 2-thio-N4-amino-5-hydroxypseudoisocytidine, 2-thio-N4-hydroxy-5-azacytidine, 2-thio-N4-hydroxypseudoisocytidine, 2-thio-N4-hydroxy-5-methylcytidine, N4-hydroxy-5-aminocytidine, 2-thio-N4-hydroxy-5-hydroxycytidine, 2-thio-N4-hydroxy-5-methyl-5-azacytidine, 2-thio-N4-hydroxy-5-amino-5-azacytidine, 2-thio-N4-hydroxy-5-hydroxy-5-azacytidine, 2-thio-N4-hydroxy-5-methylpseudoisocytidine, 2-thio-N4-hydroxy-5-aminopseudoisocytidine, 2-thio-N4-hydroxy-5-hydroxypseudoisocytidine, N6-methyladenosine, N6-aminoadenosine, N6-hydroxyadenosine, 7-deazaadenosine, 8-azaadenosine, N6-methyl-7-deazaadenosine, N6-methyl-8-azaadenosine, 7-deaza-8-azaadenosine, N6-methyl-7-deaza-8-azaadenosine, N6-amino-7-deazaadenosine, N6-amino-8-azaadenosine, N6-amino-7-deaza-8-azaadenosine, N6-hydroxyadenosine, N6-hydroxy deazaadenosine, N6-hydroxy-8-azaadenosine, N6-hydroxy-7-deaza-8-azaadenosine, 6-thioguanosine, 7-deazaguanosine, 8-azaguanosine, 6-thio-7-deazaguanosine, 6-thio-8-azaguanosine, 7-deaza-8-azaguanosine, and 6-thio-7-deaza-8-azaguanosine. In embodiments, the synthetic RNA molecule is in vitro transcribed.

In embodiments, the reprogramming is non-viral. In embodiments, the reprogramming factor is one or more of Oct4, Sox2, Klf4, c-Myc, I-Myc, Tert, Nanog, and Lin28.

### Administration and Formulations

Therapeutic treatments comprise the use of one or more routes of administration and of one or more formulations that are designed to achieve a therapeutic effect at an effective dose, while minimizing toxicity to the subject to which treatment is administered.

In various embodiments, the effective dose is an amount that substantially avoids cell toxicity in vivo. In various embodiments, the effective dose is an amount that substantially avoids an immune reaction in a human subject. For example, the immune reaction may be an immune response mediated by the innate immune system. Immune response can be monitored using markers known in the art (e.g. cytokines, interferons, TLRs). In some embodiments, the effective dose obviates the need for treatment of the human subject with immune suppressants agents (e.g. B18R) used to moderate the residual toxicity.

Upon formulation, solutions may be administered in a manner compatible with the dosage formulation and in such amount as is therapeutically effective, as described herein. The formulations may easily be administered in a variety of dosage forms such as injectable solutions and the like. For parenteral administration in an aqueous solution, for example, the solution generally is suitably buffered and the liquid diluent first rendered isotonic with, for example, sufficient saline or glucose. Such aqueous solutions may be used, for example, for intravenous, intramuscular, subcutaneous and intraperitoneal administration. Preferably, sterile aqueous media are employed as is known to those of skill in the art.

Pharmaceutical preparations may additionally comprise delivery reagents (a.k.a. “transfection reagents”, a.k.a. “vehicles”, a.k.a. “delivery vehicles”) and/or excipients. Pharmaceutically acceptable delivery reagents, excipients, and methods of preparation and use thereof, including methods for preparing and administering pharmaceutical preparations to patients (a.k.a. “subjects”) are well known in the art, and are set forth in numerous publications, including, for example, in US Patent Appl. Pub. No. US 2008/0213377, the entirety of which is incorporated herein by reference.

For example, the present compositions can be in the form of pharmaceutically acceptable salts. Such salts include those listed in, for example, J. Pharma. Sci. 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety. Non-limiting examples of pharmaceutically acceptable salts include: sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, pamoate, phenylacetate, trifluoroacetate, acrylate, chlorobenzoate, di nitrobenzoate, hydroxybenzoate, methoxybenzoate, methylbenzoate, o-acetoxybenzoate, naphthalene-2-benzoate, isobutyrate, phenylbutyrate, a-hydroxybutyrate, butyne-1,4-dicarboxylate, hexyne-1,4-dicarboxylate, caprate, caprylate, cinnamate, glycollate, heptanoate, hippurate, malate, hydroxymaleate, malonate, mandelate, mesylate, nicotinate, phthalate, teraphthalate, propiolate, propionate, phenylpropionate, sebacate, suberate, p-bromobenzenesulfonate, chlorobenzenesulfonate, ethylsulfonate, 2-hydroxyethylsulfonate, methylsulfonate, naphthalene-1-sulfonate, naphthalene-2-sulfonate, naphthalene-1,5-sulfonate, xylenesulfonate, tartarate salts, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or tri-alkylamines, dicyclohexylamine; tributyl amine; pyridine; N-methyl, N-ethylamine; diethylamine; triethylamine; mono-, bis-, or tris-(2-0H-lower alkylamines), such as mono-; bis-, or tris-(2-hydroxyethyl)amine, 2-hydroxy-tert-butylamine, or tris-(hydroxymethyl)methylamine, N,N-di-lower alkyl-N-(hydroxyl-lower alkyl)-amines, such as N,N-dimethyl-N-(2-hydroxyethyl)amine or tri-(2-hydroxyethyl)amine; N-methyl-D-glucamine; and amino acids such as arginine, lysine, and the like.

The present pharmaceutical compositions can comprise excipients, including liquids such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In one embodiment, the pharmaceutically acceptable excipients are sterile when administered to a subject. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent described herein, if desired, can also comprise minor amounts of wetting or emulsifying agents, or pH buffering agents.

In embodiments, the composition is formulated for one or more of intrathecal, intra-lesional, intra-coronary, intravenous (IV), intra-articular, intramuscular, and intra-endobronchial administration and administration via intrapancreatic endovascular injection, intra-nucleus pulposus, lumbar puncture, intra-myocardium, transendocardium, intra-fistula tract, intermedullary space, intradural space and leg injection.

In embodiments, the composition is formulated for infusion. In some embodiments, the composition is formulated for infusion, wherein the composition is delivered to the bloodstream of a subject or patient through a needle in a vein of the subject or patient through a peripheral line, a central line, a tunneled line, an implantable port, and/or a catheter. In some embodiments, the subject or patient may also receive supportive medications or treatments, such as hydration, by infusion. In some embodiments, the composition is formulated for intravenous infusion. In some embodiments, the infusion is continuous infusion, secondary intravenous therapy (IV), and/or IV push. In some embodiments, the infusion of the composition may be administered through the use of equipment selected from one or more of an infusion pump, hypodermic needle, drip chamber, peripheral cannula, and pressure bag.

In some embodiments, the composition is formulated for inhalation. In some embodiments, the inhalation comprises breathing in the composition directly into the lungs. In some embodiments, the inhalation of the composition may be administered through the use of one or more of a metered-dose inhaler, dry powder inhaler, nebulizer, and soft mist inhaler.

### Definitions

By “synthetic RNA molecule” is meant an RNA molecule that is produced outside of a cell or that is produced inside of a cell using bioengineering, by way of non-limiting example, an RNA molecule that is produced in an in vitro-transcription reaction, an RNA molecule that is produced by direct chemical synthesis or an RNA molecule that is produced in a genetically-engineered E. coli cell.

By “medium” is meant a solvent or a solution comprising a solvent and a solute, by way of non-limiting example, Dulbecco's Modified Eagle's Medium (DMEM), DMEM+10% fetal bovine serum (FBS), saline or water.

By “transfection medium” is meant a medium that can be used for transfection, by way of non-limiting example, Dulbecco's Modified Eagle's Medium (DMEM), DMEM/F12, saline or water.

By “Oct4 protein” is meant a protein that is encoded by the POU5F1 gene, or a natural or engineered variant, family-member, orthologue, fragment or fusion construct thereof, by way of non-limiting example, human Oct4 protein (SEQ ID NO: 1), mouse Oct4 protein, Oct1 protein, a protein encoded by POU5F1 pseudogene 2, a DNA-binding domain of Oct4 protein or an Oct4-GFP fusion protein. In some embodiments the Oct4 protein comprises an amino acid sequence that has at least 70% identity with SEQ ID NO: 1, or in other embodiments, at least 75%, 80%, 85%, 90%, or 95% identity with SEQ ID NO: 1. In some embodiments, the Oct4 protein comprises an amino acid sequence having from 1 to 20 amino acid insertions, deletions, or substitutions (collectively) with respect to SEQ ID NO: 1. Or in other embodiments, the Oct4 protein comprises an amino acid sequence having from 1 to 15 or from 1 to 10 amino acid insertions, deletions, or substitutions (collectively) with respect to SEQ ID NO: 1.

SEQ ID NO: 1 is:
Met Ala Gly His Leu Ala Ser Asp Phe Ala
Phe Ser Pro Pro Pro Gly Gly Gly Gly Asp
Gly Pro Gly Gly Pro Glu Pro Gly Trp Val
Asp Pro Arg Thr Trp Leu Ser Phe Gln Gly
Pro Pro Gly Gly Pro Gly Ile Gly Pro Gly
Val Gly Pro Gly Ser Glu Val Trp Gly Ile
Pro Pro Cys Pro Pro Pro Tyr Glu Phe Cys
Gly Gly Met Ala Tyr Cys Gly Pro Gln Val
Gly Val Gly Leu Val Pro Gln Gly Gly Leu
Glu Thr Ser Gln Pro Glu Gly Glu Ala Gly
Val Gly Val Glu Ser Asn Ser Asp Gly Ala
Ser Pro Glu Pro Cys Thr Val Thr Pro Gly
Ala Val Lys Leu Glu Lys Glu Lys Leu Glu
Gln Asn Pro Glu Glu Ser Gln Asp Ile Lys
Ala Leu Gln Lys Glu Leu Glu Gln Phe Ala
Lys Leu Leu Lys Gln Lys Arg Ile Thr Leu
Gly Tyr Thr Gln Ala Asp Val Gly Leu Thr
Leu Gly Val Leu Phe Gly Lys Val Phe Ser
Gln Thr Thr Ile Cys Arg Phe Glu Ala Leu
Gln Leu Ser Phe Lys Asn Met Cys Lys Leu
Arg Pro Leu Leu Gln Lys Trp Val Glu Glu
Ala Asp Asn Asn Glu Asn Leu Gln Glu Ile
Cys Lys Ala Glu Thr Leu Val Gln Ala Arg
Lys Arg Lys Arg Thr Ser Ile Glu Asn Arg
Val Arg Gly Asn Leu Glu Asn Leu Phe Leu
Gln Cys Pro Lys Pro Thr Leu Gln Gln Ile
Ser His Ile Ala Gln Gln Leu Gly Leu Glu
Lys Asp Val Val Arg Val Trp Phe Cys Asn
Arg Arg Gln Lys Gly Lys Arg Ser Ser Ser
Asp Tyr Ala Gln Arg Glu Asp Phe Glu Ala
Ala Gly Ser Pro Phe Ser Gly Gly Pro Val
Ser Phe Pro Leu Ala Pro Gly Pro His Phe
Gly Thr Pro Gly Tyr Gly Ser Pro His Phe
Thr Ala Leu Tyr Ser Ser Val Pro Phe Pro
Glu Gly Glu Ala Phe Pro Pro Val Ser Val
Thr Thr Leu Gly Ser Pro Met His Ser Asn

By “Sox2 protein” is meant a protein that is encoded by the SOX2 gene, or a natural or engineered variant, family-member, orthologue, fragment or fusion construct thereof, by way of non-limiting example, human Sox2 protein (SEQ ID NO: 2), mouse Sox2 protein, a DNA-binding domain of Sox2 protein or a Sox2-GFP fusion protein. In some embodiments the Sox2 protein comprises an amino acid sequence that has at least 70% identity with SEQ ID NO: 2, or in other embodiments, at least 75%, 80%, 85%, 90%, or 95% identity with SEQ ID NO: 2. In some embodiments, the Sox2 protein comprises an amino acid sequence having from 1 to 20 amino acid insertions, deletions, or substitutions (collectively) with respect to SEQ ID NO: 2. Or in other embodiments, the Sox2 protein comprises an amino acid sequence having from 1 to 15 or from 1 to 10 amino acid insertions, deletions, or substitutions (collectively) with respect to SEQ ID NO: 2.

SEQ ID NO: 2 is:
Met Tyr Asn Met Met Glu Thr Glu Leu Lys
Pro Pro Gly Pro Gln Gln Thr Ser Gly Gly
Gly Gly Gly Asn Ser Thr Ala Ala Ala Ala
Gly Gly Asn Gln Lys Asn Ser Pro Asp Arg
Val Lys Arg Pro Met Asn Ala Phe Met Val
Trp Ser Arg Gly Gln Arg Arg Lys Met Ala
Gln Glu Asn Pro Lys Met His Asn Ser Glu
Ile Ser Lys Arg Leu Gly Ala Glu Trp Lys
Leu Leu Ser Glu Thr Glu Lys Arg Pro Phe
Ile Asp Glu Ala Lys Arg Leu Arg Ala Leu
His Met Lys Glu His Pro Asp Tyr Lys Tyr
Arg Pro Arg Arg Lys Thr Lys Thr Leu Met
Lys Lys Asp Lys Tyr Thr Leu Pro Gly Gly
Leu Leu Ala Pro Gly Gly Asn Ser Met Ala
Ser Gly Val Gly Val Gly Ala Gly Leu Gly
Ala Gly Val Asn Gln Arg Met Asp Ser Tyr
Ala His Met Asn Gly Trp Ser Asn Gly Ser
Tyr Ser Met Met Gln Asp Gln Leu Gly Tyr
Pro Gln His Pro Gly Leu Asn Ala His Gly
Ala Ala Gln Met Gln Pro Met His Arg Tyr
Asp Val Ser Ala Leu Gln Tyr Asn Ser Met
Thr Ser Ser Gln Thr Tyr Met Asn Gly Ser
Pro Thr Tyr Ser Met Ser Tyr Ser Gln Gln
Gly Thr Pro Gly Met Ala Leu Gly Ser Met
Gly Ser Val Val Lys Ser Glu Ala Ser Ser
Ser Pro Pro Val Val Thr Ser Ser Ser His
Ser Arg Ala Pro Cys Gln Ala Gly Asp Leu
Arg Asp Met Ile Ser Met Tyr Leu Pro Gly
Ala Glu Val Pro Glu Pro Ala Ala Pro Ser
Arg Leu His Met Ser Gln His Tyr Gln Ser
Gly Pro Val Pro Gly Thr Ala Ile Asn Gly
Thr Leu Pro Leu Ser His Met

By “Klf4 protein” is meant a protein that is encoded by the KLF4 gene, or a natural or engineered variant, family-member, orthologue, fragment or fusion construct thereof, by way of non-limiting example, human Klf4 protein (SEQ ID NO: 3), mouse Klf4 protein, a DNA-binding domain of Klf4 protein or a Klf4-GFP fusion protein. In some embodiments the Klf4 protein comprises an amino acid sequence that has at least 70% identity with SEQ ID NO: 3, or in other embodiments, at least 75%, 80%, 85%, 90%, or 95% identity with SEQ ID NO: 13. In some embodiments, the Klf4 protein comprises an amino acid sequence having from 1 to 20 amino acid insertions, deletions, or substitutions (collectively) with respect to SEQ ID NO: 3. Or in other embodiments, the Klf4 protein comprises an amino acid sequence having from 1 to 15 or from 1 to 10 amino acid insertions, deletions, or substitutions (collectively) with respect to SEQ ID NO: 3.

SEQ ID NO: 3 is:
Met Arg Gln Pro Pro Gly Glu Ser Asp Met
Ala Val Ser Asp Ala Leu Leu Pro Ser Phe
Ser Thr Phe Ala Ser Gly Pro Ala Gly Arg
Glu Lys Thr Leu Arg Gln Ala Gly Ala Pro
Asn Asn Arg Trp Arg Glu Glu Leu Ser His
Met Lys Arg Leu Pro Pro Val Leu Pro Gly
Arg Pro Tyr Asp Leu Ala Ala Ala Thr Val
Ala Thr Asp Leu Glu Ser Gly Gly Ala Gly
Ala Ala Cys Gly Gly Ser Asn Leu Ala Pro
Leu Pro Arg Arg Glu Thr Glu Glu Phe Asn
Asp Leu Leu Asp Leu Asp Phe Ile Leu Ser
Asn Ser Leu Thr His Pro Pro Glu Ser Val
Ala Ala Thr Val Ser Ser Ser Ala Ser Ala
Ser Ser Ser Ser Ser Pro Ser Ser Ser Gly
Pro Ala Ser Ala Pro Ser Thr Cys Ser Phe
Thr Tyr Pro Ile Arg Ala Gly Asn Asp Pro
Gly Val Ala Pro Gly Gly Thr Gly Gly Gly
Leu Leu Tyr Gly Arg Glu Ser Ala Pro Pro
Pro Thr Ala Pro Phe Asn Leu Ala Asp Ile
Asn Asp Val Ser Pro Ser Gly Gly Phe Val
Ala Glu Leu Leu Arg Pro Glu Leu Asp Pro
Val Tyr Ile Pro Pro Gln Gln Pro Gln Pro
Pro Gly Gly Gly Leu Met Gly Lys Phe Val
Leu Lys Ala Ser Leu Ser Ala Pro Gly Ser
Glu Tyr Gly Ser Pro Ser Val Ile Ser Val
Ser Lys Gly Ser Pro Asp Gly Ser His Pro
Val Val Val Ala Pro Tyr Asn Gly Gly Pro
Pro Arg Thr Cys Pro Lys Ile Lys Gln Glu
Ala Val Ser Ser Cys Thr His Leu Gly Ala
Gly Pro Pro Leu Ser Asn Gly His Arg Pro
Ala Ala His Asp Phe Pro Leu Gly Arg Gln
Leu Pro Ser Arg Thr Thr Pro Thr Leu Gly
Leu Glu Glu Val Leu Ser Ser Arg Asp Cys
His Pro Ala Leu Pro Leu Pro Pro Gly Phe
His Pro His Pro Gly Pro Asn Tyr Pro Ser
Phe Leu Pro Asp Gln Met Gln Pro Gln Val
Pro Pro Leu His Tyr Gln Glu Leu Met Pro
Pro Gly Ser Cys Met Pro Glu Glu Pro Lys
Pro Lys Arg Gly Arg Arg Ser Trp Pro Arg
Lys Arg Thr Ala Thr His Thr Cys Asp Tyr
Ala Gly Cys Gly Lys Thr Tyr Thr Lys Ser
Ser His Leu Lys Ala His Leu Arg Thr His
Thr Gly Glu Lys Pro Tyr His Cys Asp Trp
Asp Gly Cys Gly Trp Lys Phe Ala Arg Ser
Asp Glu Leu Thr Arg His Tyr Arg Lys His
Thr Gly His Arg Pro Phe Gln Cys Gln Lys
Cys Asp Arg Ala Phe Ser Arg Ser Asp His
Leu Ala Leu His Met Lys Arg His Phe

By “c-Myc protein” is meant a protein that is encoded by the MYC gene, or a natural or engineered variant, family-member, orthologue, fragment or fusion construct thereof, by way of non-limiting example, human c-Myc protein (SEQ ID NO: 4), mouse c-Myc protein, I-Myc protein, c-Myc (T58A) protein, a DNA-binding domain of c-Myc protein or a c-Myc-GFP fusion protein. In some embodiments the c-Myc protein comprises an amino acid sequence that has at least 70% identity with SEQ ID NO: 4, or in other embodiments, at least 75%, 80%, 85%, 90%, or 95% identity with SEQ ID NO: 4. In some embodiments, the c-Myc protein comprises an amino acid having from 1 to 20 amino acid insertions, deletions, or substitutions (collectively) with respect to SEQ ID NO: 4. Or in other embodiments, the c-Myc protein comprises an amino acid sequence having from 1 to 15 or from 1 to 10 amino acid insertions, deletions, or substitutions (collectively) with respect to SEQ ID NO: 4.

SEQ ID NO: 4 is:
Met Asp Phe Phe Arg Val Val Glu Asn Gln
Gln Pro Pro Ala Thr Met Pro Leu Asn Val
Ser Phe Thr Asn Arg Asn Tyr Asp Leu Asp
Tyr Asp Ser Val Gln Pro Tyr Phe Tyr Cys
Asp Glu Glu Glu Asn Phe Tyr Gln Gln Gln
Gln Gln Ser Glu Leu Gln Pro Pro Ala Pro
Ser Glu Asp Ile Trp Lys Lys Phe Glu Leu
Leu Pro Thr Pro Pro Leu Ser Pro Ser Arg
Arg Ser Gly Leu Cys Ser Pro Ser Tyr Val
Ala Val Thr Pro Phe Ser Leu Arg Gly Asp
Asn Asp Gly Gly Gly Gly Ser Phe Ser Thr
Ala Asp Gln Leu Glu Met Val Thr Glu Leu
Leu Gly Gly Asp Met Val Asn Gln Ser Phe
Ile Cys Asp Pro Asp Asp Glu Thr Phe Ile
Lys Asn Ile Ile Ile Gln Asp Cys Met Trp
Ser Gly Phe Ser Ala Ala Ala Lys Leu Val
Ser Glu Lys Leu Ala Ser Tyr Gln Ala Ala
Arg Lys Asp Ser Gly Ser Pro Asn Pro Ala
Arg Gly His Ser Val Cys Ser Thr Ser Ser
Leu Tyr Leu Gln Asp Leu Ser Ala Ala Ala
Ser Glu Cys Ile Asp Pro Ser Val Val Phe
Pro Tyr Pro Leu Asn Asp Ser Ser Ser Pro
Lys Ser Cys Ala Ser Gln Asp Ser Ser Ala
Phe Ser Pro Ser Ser Asp Ser Leu Leu Ser
Ser Thr Glu Ser Ser Pro Gln Gly Ser Pro
Glu Pro Leu Val Leu His Glu Glu Thr Pro
Pro Thr Thr Ser Ser Asp Ser Glu Glu Glu
Gln Glu Asp Glu Glu Glu Ile Asp Val Val
Ser Val Glu Lys Arg Gln Ala Pro Gly Lys
Arg Ser Glu Ser Gly Ser Pro Ser Ala Gly
Gly His Ser Lys Pro Pro His Ser Pro Leu
Val Leu Lys Arg Cys His Val Ser Thr His
Gln His Asn Tyr Ala Ala Pro Pro Ser Thr
Arg Lys Asp Tyr Pro Ala Ala Lys Arg Val
Lys Leu Asp Ser Val Arg Val Leu Arg Gln
Ile Ser Asn Asn Arg Lys Cys Thr Ser Pro
Arg Ser Ser Asp Thr Glu Glu Asn Val Lys
Arg Arg Thr His Asn Val Leu Glu Arg Gln
Arg Arg Asn Glu Leu Lys Arg Ser Phe Phe
Ala Leu Arg Asp Gln Ile Pro Glu Leu Glu
Asn Asn Glu Lys Ala Pro Lys Val Val Ile
Leu Lys Lys Ala Thr Ala Tyr Ile Leu Ser
Val Gln Ala Glu Glu Gln Lys Leu Ile Ser
Glu Glu Asp Leu Leu Arg Lys Arg Arg Glu
Gln Leu Lys

This invention is further illustrated by the following non-limiting examples.

### SEQUENCES

In embodiments, e.g. those involving further engineering the MSC to express and/or secrete a soluble protein, the MSC cells is transduced or electroporated with one or more of the following sequences, or a sequence having at about 95%, or at least about 97%, or at least about 98% identity thereto, or a codon-optimized version thereof.

>TRAIL
(SEQ ID NO: 5)
ATGGCCATGATGGAAGTGCAGGGTGGGCCCTCCCT
CGGGCAGACGTGCGTGCTCATAGTCATTTTCACCG
TGCTCCTCCAGAGCCTGTGCGTCGCCGTCACGTAC
GTGTACTTCACCAACGAGCTGAAGCAGATGCAGGA
CAAGTACAGCAAGTCGGGGATCGCCTGCTTCCTGA
AGGAGGACGACTCCTACTGGGACCCGAACGACGAG
GAGTCCATGAACTCCCCGTGCTGGCAGGTCAAGTG
GCAGCTGCGGCAGCTGGTCCGCAAGATGATCCTCC
GGACCAGCGAGGAGACTATCAGCACCGTCCAGGAG
AAGCAGCAGAACATCTCCCCGCTCGTCCGGGAGCG
AGGTCCCCAGCGGGTAGCGGCTCATATCACCGGCA
CCCGCGGGAGGTCGAACACGCTCAGCTCGCCGAAC
TCCAAGAACGAGAAGGCCCTCGGCCGCAAGATCAA
CTCCTGGGAGAGCTCGCGGAGCGGGCACTCGTTCC
TGTCGAACCTGCATCTGCGGAACGGCGAGCTCGTG
ATCCACGAGAAGGGCTTCTACTACATCTACAGCCA
GACCTACTTCCGGTTCCAGGAGGAGATCAAGGAGA
ACACCAAGAACGACAAGCAGATGGTGCAGTACATC
TACAAGTACACCAGCTACCCCGACCCGATCCTCCT
GATGAAGTCCGCCCGCAACAGCTGCTGGAGCAAGG
ACGCGGAGTACGGCCTGTACAGCATCTACCAGGGC
GGCATCTTCGAGCTCAAGGAGAACGACCGCATCTT
CGTGAGCGTGACGAACGAGCACCTGATCGACATGG
ACCACGAGGCCTCCTTCTTCGGGGCGTTCCTGGTC
GGTTGA
>FLT3LG
(SEQ ID NO: 6)
ATGACAGTGCTGGCGCCCGCCTGGTCCCCCACAAC
CTACCTCCTCCTGCTGCTGTTGCTGTCGTCGGGCC
TGAGCGGGACGCAGGACTGCTCGTTCCAGCACAGC
CCGATCTCCTCCGACTTCGCGGTCAAGATCCGGGA
GCTGAGCGACTACCTGTTGCAGGACTACCCCGTCA
CCGTCGCCTCCAACCTGCAGGACGAGGAGCTCTGC
GGTGGCTTGTGGCGTCTGGTGTTGGCTCAGCGCTG
GATGGAGCGGCTCAAGACGGTCGCGGGGTCCAAGA
TGCAGGGCCTGCTCGAGCGCGTGAACACGGAGATC
CACTTCGTCACCAAGTGCGCCTTCCAGCCGCCACC
CAGCTGCCTGCGGTTCGTCCAGACCAACATCTCCC
GCCTCCTGCAGGAGACATCCGAGCAGCTGGTGGCG
CTGAAGCCCTGGATCACGCGCCAGAACTTCTCCCG
ATGCCTGGAGCTGCAGTGCCAGCCTGACTCCTCAA
CCCTGCCACCTCCATGGAGTCCCCGGCCTTTGGAG
GCCACCGCGCCCACTGCTCCGCAGCCCCCACTCCT
CTTGCTACTCCTGCTCCCCGTAGGCCTTCTACTGC
TGGCCGCAGCCTGGTGCCTACATTGGCAAAGAACG
CGGCGACGTACACCCCGCCCTGGGGAGCAGGTGCC
CCCAGTCCCCAGTCCCCAGGACCTGCTGCTTGTGG
AGCACTGA
>IL7-(G4S)2-IL15
(SEQ ID NO: 7)
ATGTTCCACGTGTCGTTCCGGTACATCTTCGGCCT
CCCGCCGCTCATCCTGGTCCTGCTCCCCGTGGCGT
CCTCGGACTGCGACATCGAGGGCAAGGACGGGAAG
CAGTACGAGTCGGTGCTGATGGTGTCCATCGACCA
GCTGCTCGACTCCATGAAGGAGATCGGGTCGAACT
GCCTCAACAACGAGTTCAACTTCTTCAAGCGCCAC
ATCTGCGACGCGAACAAGGAGGGGATGTTCCTCTT
CCGGGCGGCCCGCAAGCTCCGGCAGTTCCTCAAGA
TGAACAGCACGGGGGACTTCGACCTGCACCTGCTG
AAGGTGTCGGAGGGCACCACCATCCTCCTCAACTG
CACCGGCCAGGTCAAGGGCCGGAAGCCCGCCGCCC
TGGGCGAGGCGCAGCCCACGAAGTCCCTCGAGGAG
AACAAGTCCCTCAAGGAGCAGAAGAAGCTCAACGA
CCTGTGCTTCCTCAAGCGGCTCCTCCAGGAGATCA
AGACCTGCTGGAACAAGATCCTCATGGGCACCAAG
GAGCACGGGGGCGGCGGGAGCGGCGGGGGTGGCTC
CAACTGGGTGAACGTCATCTCGGACCTCAAGAAGA
TCGAGGACCTGATCCAGAGCATGCACATCGACGCC
ACGCTCTACACCGAGTCGGACGTCCACCCGTCGTG
CAAGGTCACCGCCATGAAGTGCTTCCTCCTGGAGC
TGCAGGTCATCTCGCTGGAGTCCGGCGACGCCAGC
ATCCACGACACGGTCGAGAACCTCATCATCCTCGC
GAACAACTCCCTCAGCTCGAACGGCAACGTGACGG
AGAGCGGGTGCAAGGAGTGCGAGGAGCTGGAGGAG
AAGAACATCAAGGAGTTCCTGCAGAGCTTCGTGCA
CATCGTGCAGATGTTCATCAACACCAGCTGA.

### EXAMPLES

### Example 1: Generation of MSCs

Induced pluripotent stem cells (iPSCs) were obtained and MSCs were generated via cell reprogramming with non-immunogenic messenger RNA (mRNA) encoding one or more reprogramming factors in a defined, animal-component-free process.

iPSCs were generated from adult human dermal fibroblasts using a high-efficiency, immunosuppressant-free mRNA-based protocol. iPSCs were then differentiated into MSCs using a 21-day high-yield monolayer protocol. rtPCR analysis showed downregulation of Nanog and Oct4 and upregulation of CD73 and CD105 in the differentiated MSCs. Multipotency was confirmed by differentiation into adipocytes, osteoblasts, and chondrocytes. iPSC MSCs had approximately 13 kb long telomeres compared to 7 kb long telomeres of bone marrow-derived MSCs (BM MSCs), as measured by Southern analysis of terminal restriction fragments. When serially passaged, iPSC MSCs underwent >70 population doublings before senescence, compared to <20 population doublings for BM MSCs. See FIG. 1.

### Example 2: Gene Expression and Protein Secretion Profiles of MSCs

The MSCs of Example 1 were characterized for gene and secreted protein signatures.

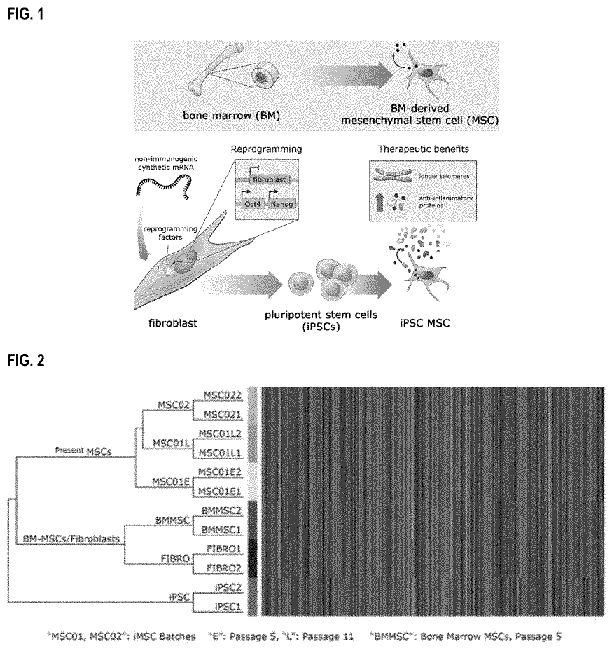

FIG. 2 shows a gene expression-based cell signature. Donor-derived MSCs (BM-MSCs) mimic immunologically inert fibroblasts. By contrast, the present MSCs cluster together (multiple batches, early and late passages). This indicates that the present MSCs are immunologically active.

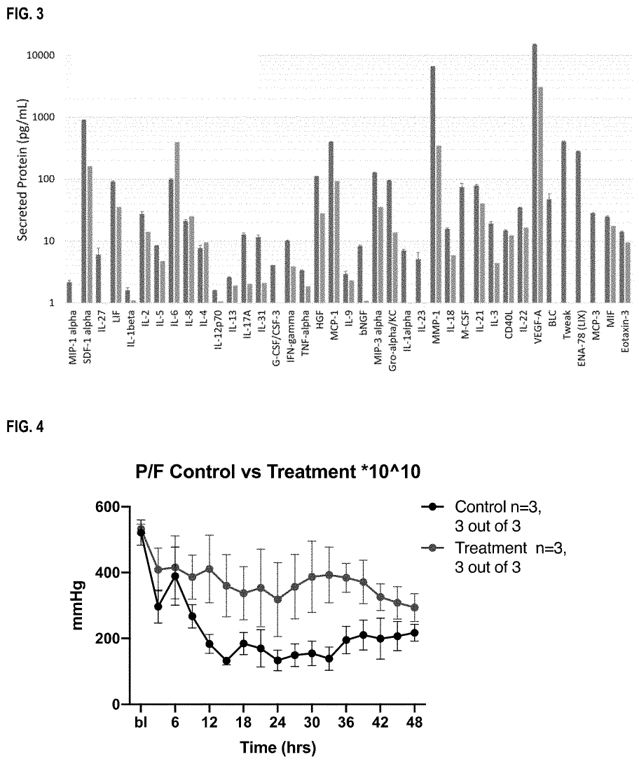

FIG. 3 shows a protein secretion-based cell signature. The present MSCs show increased secretion of MIP-1 alpha, SDF-1 alpha, IL-27, LIF, IL-1 beta, IL-2, IL-5, IL-12p70, IL-13, IL-17A, IL-31, G-CSF/CSF-3, IFN-gamma, TNF-alpha, HGF, MCP-1, IL-9, bNGF, MIP-3 alpha, Gro-alpha/KC, IL-1alpha, IL-23, MMP-1, IL-18, M-CSF, IL-21, M-CSF, IL-21, CD40L, IL-22, VEGF-A, BLC, Tweak, ENA-78 (LIX), MCP-3, MIF, and Eotaxin-3 relative to a bone marrow-derived MSCs The present MSCs show decreased secretion of IL-6, IL-8, and IL-4 relative to a bone marrow-derived MSCs. See also Table 1 for quantitation of secretion data.

### Example 3: In Vivo Efficacy in a ARDS Model

In vivo efficacy of the present MSCs was evaluated in a sheep acute respiratory distress syndrome (ARDS) model. In this ovine model of severe ARDS the animals receiving MSCs demonstrated clear improvement over control animals in several parameters, including oxygenation; systemic blood pressure; and lung/alveolar permeability.

Adult Merino female sheep weighing 30 to 40 kg were obtained. For the studies, 20 female sheep total; N=8 sheep/group were used. The study was randomized and occurred in a large animal intensive care setting. The study duration for each sheep was 48 hr and the dosing regimen was 2 doses of either MSCs (10 million cells/kg) or control (PlasmaLite A). NC-MSCs or control were be administered at 1 hr and 24 hr following acute lung injury. The MSCs or control were administered by IV infusion (central line) for 1 hr starting 1 hr after the injury and dosing was repeated in a similar fashion at 24 hr following injury.

After, at least, 14 days quarantine time, sheep were surgically prepared for measurement of heart rate, systemic arterial pressure, pulmonary arterial pressure (including cardiac output), pulmonary arterial wedge pressure, left atrial pressure and central venous pressure. For the pulmonary transvascular fluid flux assessment, the efferent vessel of caudal mediastinal lymph node was cannulated in some sheep. These instrumental procedures were performed under isoflurane anesthesia via endotracheal tube.

After 5 to 7 days of surgical recovery, baseline measurements of respiratory and hemodynamic variables were taken twice with a 30 min interval in awake, unanesthetized sheep. Baseline variables include cardiopulmonary hemodynamics and arterial and venous blood gas analysis.

Under deep anesthesia (10 to 15 mg/kg of IV ketamine) and analgesia (buprenorphineSR 0.1 to 0.27 mg/kg), a tracheostomy tube was placed and anesthesia was supported by inhaled isoflurane (2 to 5%). When, the coughing reflex was inhibited, Pseudomonas aeruginosa was instilled into the lungs by bronchoscope. Live Pseudomonas aeruginosa (2.5 to 3.5×1011 CFU total) was suspended in 30 mL of 0.9% normal saline and instilled by direct vision into the right middle lobe and right lower lobe and the left lower lobe of the lung (10 ml each) by fiberoptic bronchoscopy. After the bacterial instillation, anesthesia was continued with isoflurane for approximately 10 minutes to prevent the premature expectoration of the instilled bacteria by coughing.

Additionally, a Foley catheter was inserted into the bladder and baseline urine samples were taken.

After the injury, sheep were placed on mechanical ventilator (Avea APVcmv mode: RR 20 b/m, TV 12 mL/kg, PEEP 5 cmH₂O). Immediately, after the placing the sheep on the ventilator, pulmonary mechanic baseline variables, peak and pause airway pressures, and compliance were taken.

Study assessments following treatment were performed in the same manner as baseline variables. Study assessments include hemodynamic monitoring, pulmonary gas exchange, and complete blood cell count, lung lymph flow and its protein, among others.

After euthanasia (performed either upon euthanasia criteria being reached or completion of the 48-hr study period), lung tissue was removed and a bronchoalveolar lavage was performed in the left lung.

The PaO₂/FiO₂ ratio is the ratio of arterial oxygen partial pressure (PaO₂ in mmHg) to fractional inspired oxygen (FiO₂ expressed as a fraction, not a percentage). FIG. 4 shows, inter alia, improved oxygenation in the MSC treatment group, as compared to controls.

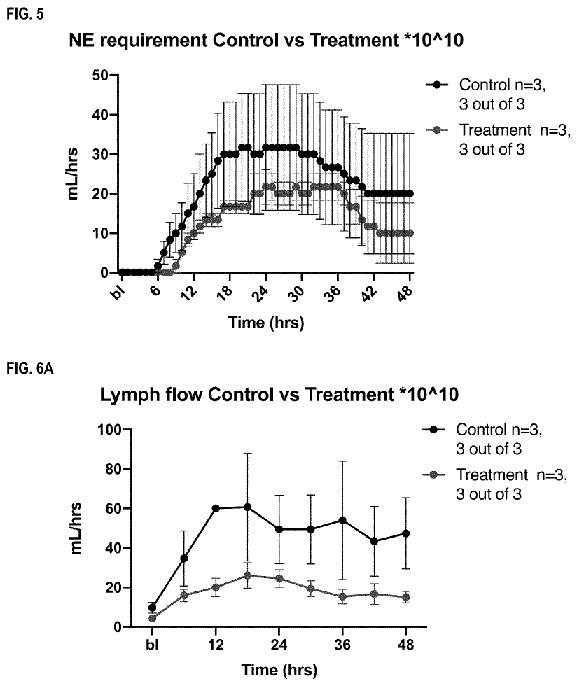

FIG. 5 shows the norepinephrine requirement in the ARDS model sheep that received the present MSCs versus control. This data indicates improved systemic blood pressure, e.g. less shock/less pressor support, in the MSC treatment group, as compared to controls.

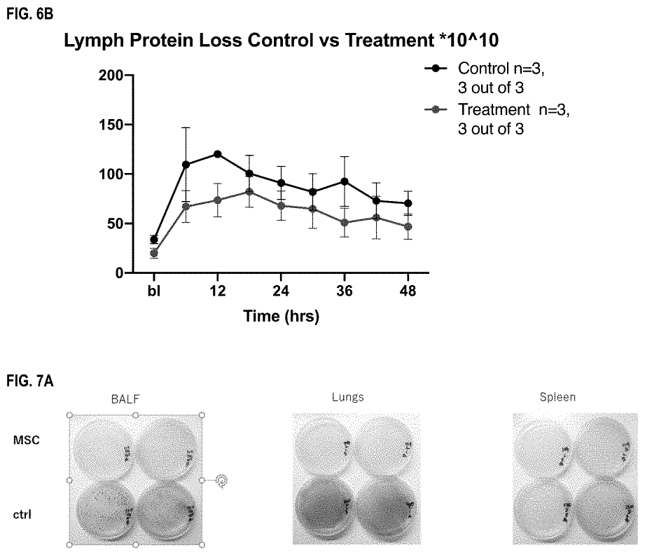

FIGS. 6A-B show reduced lung vascular injury in the MSC treatment group, as compared to controls. Specifically FIG. 6A shows pulmonary lymph flow. FIG. 6B shows pulmonary lymph protein loss. lung/alveolar permeability. Accordingly, FIG. 6A-B shows, inter alia, a decrease in production of excess lung lymph flow and associated protein loss.

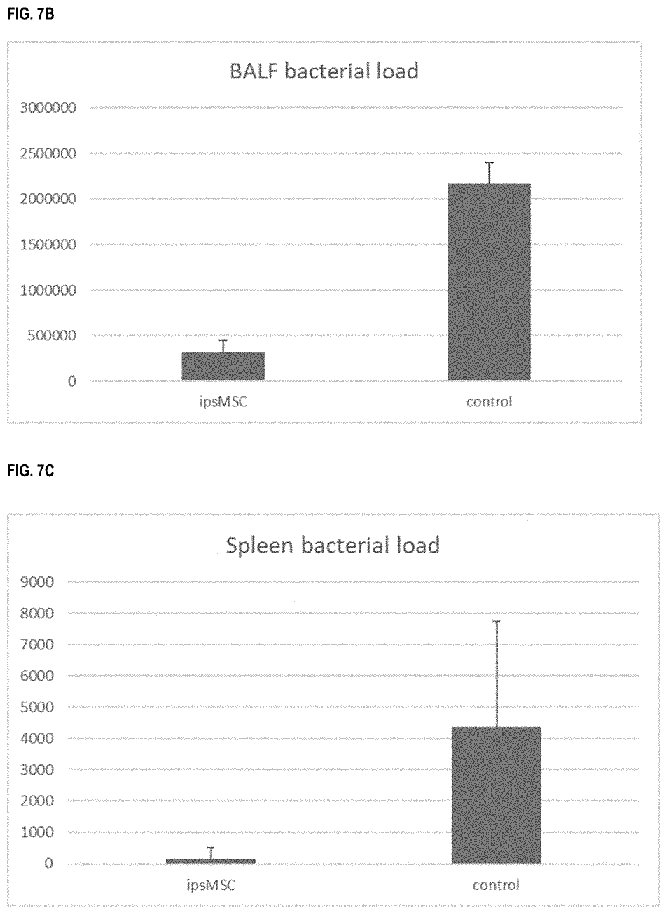

FIGS. 7A-C show tissue bacterial count in the MSC treatment group, as compared to controls in the ARDS model sheep. FIG. 7A shows bacterial growth in MSC versus control settings for bronchoalveolar lavage fluid (BALF), lungs and spleen. FIG. 7B shows BALF bacterial load counts in the MSC treatment group, as compared to controls. FIG. 7C shows spleen bacterial load counts in the MSC treatment group, as compared to controls.

### Example 4: MSC Secretion of Agents for Oncology Uses

MSCs were engineered to express and secrete desirable proteins for, e.g. use in cancer treatments.

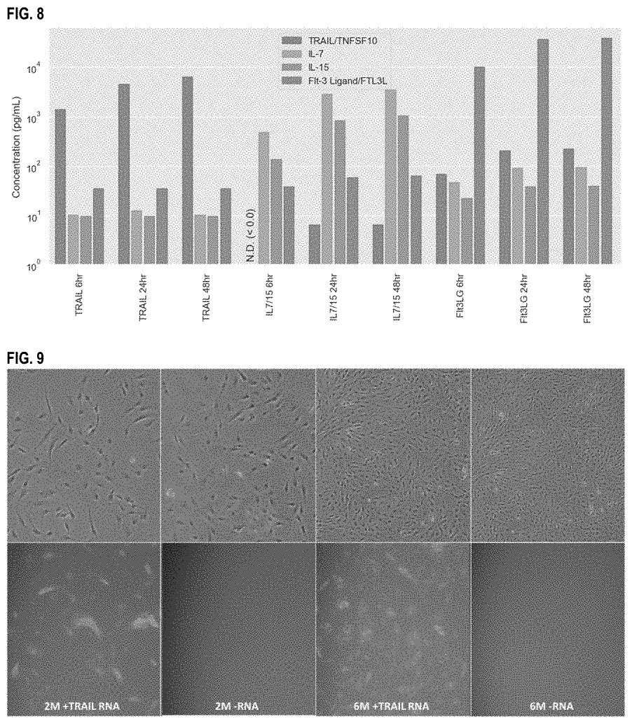

FIG. 8 shows cytokine expression in mesenchymal stem cells (MSCs) following mRNA electroporation.

200,000 Mesenchymal stem cells were electroporated with 2 μg RNA encoding: TRAIL, IL7-(G₄S)₂-IL15 or Flt3 ligand. Media was sampled at 6, 24 h and 48 h following electroporation and the protein concentration was measured using the Luminex MagPix system with a custom panel.

FIG. 9 shows TRAIL expression in MSCs following electroporation and freezing. Two million (2M) and six million (6M) MSCs derived from induced pluripotent stem cells were electroporated with mRNA encoding TRAIL (1 μg RNA/100,000 cells) and frozen immediately. Cells were thawed, plated, and left for 24 h before fixing and staining using a rabbit anti-hTRAIL primary antibody (C92B9, Cell Signaling Technology) and AlexaFluor 488 donkey anti-rabbit secondary antibody (A21206, Invitrogen).

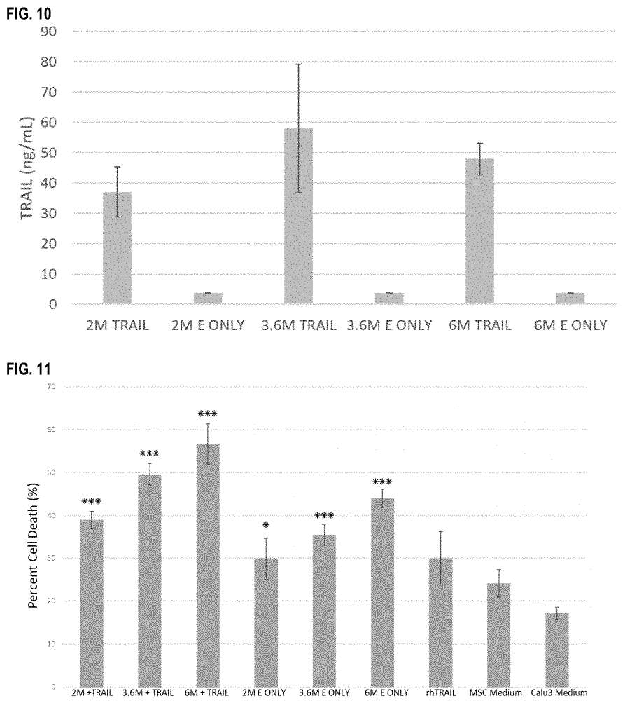

FIG. 10 shows TRAIL secretion following electroporation of iPSC-derived MSCs with RNA encoding TRAIL. 2×10⁶, 3.6×10⁶, or 6×10⁶ cells were electroporated with 1 μg RNA/100,000 cells and the concentration of TRAIL in the medium was measured 24 h after electroporation via ELISA (CD253, Abcam). “E ONLY” indicates electroporation of MSCs without RNA added.

FIG. 11 shows cancer cell killing with MSC supernatant. Calu3 human lung adenocarcinoma cells were growth to approximately 80% confluency and dissociated using TrypLE Select CTS. Cells were pelleted and resuspended in 400 μL supernatant from MSCs electroporated with the indicated conditions, recombinant hTRAIL (310-04, Peprotech) in MSC culture medium (Mesencult, Stem Cell Technologies), MSC culture medium, or Calu3 culture medium (aMEM+10% FBS). Cells were plated for 24 h, dissociated with TrypLE Select CTS, stained with Annexin V and propium iodide to identify apoptotic and necrotic cells. Cells were counted via flow cytometry. Stars indicate significance relative to Calu3 medium samples determined using an unpaired two-sided t-test. *P<0.05; **P<0.01; ***P<0.005

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

What is claimed is:

1. A method of making a composition comprising a therapeutic cell, comprising:
(a) reprogramming an induced pluripotent stem cell (iPSC) into a mesenchymal stem cell (MSC), the reprogramming comprising contacting the iPSC with one or more synthetic RNA molecules encoding a reprogramming factor;
(b) assaying the MSC for a protein secretion signature, the protein secretion signature comprising an increased secretion of one or more proteins selected from MIP-1 alpha, SDF-1 alpha, IL-27, LIF, IL-1 beta, IL-2, IL-5, IL-12p70, IL-13, IL-17A, IL-31, G-CSF/CSF-3, IFN-gamma, TNF-alpha, HGF, MCP-1, IL-9, bNGF, MIP-3 alpha, Gro-alpha/KC, IL-1alpha, IL-23, MMP-1, IL-18, M-CSF, IL-21, M-CSF, IL-21, CD40L, IL-22, VEGF-A, BLC, Tweak, ENA-78 (LIX), MCP-3, MIF, and Eotaxin-3 and/or a decreased secretion of one or more proteins selected from IL-6, IL-8, and IL-4,
wherein the increased and/or decreased secretion is relative to a bone marrow-derived MSC; and
(c) formulating the MSC substantially having the protein secretion signature for therapy.

2. The method of claim 1, wherein the iPSC is derived from a human.

3. The method of claim 2, wherein the iPSC is derived from a subject who is not intended to receive the therapy.

4. The method of any one of the preceding claims, wherein the iPSC is allogeneic to a patient intended to receive the therapy.

5. The method of any one of the preceding claims, wherein the iPSC is from a master cell bank.

6. The method of any one of the preceding claims, wherein the MSC is characterized by low or reduced inflammation.

7. The method of any one of the preceding claims, wherein the MSC is characterized by low or reduced immunogenicity.

8. The method of any one of the preceding claims, wherein the MSC is self-renewing.

9. The method of any one of the preceding claims, wherein the MSC is multipotent.

10. The method of any one of the preceding claims, wherein the MSC is immune inhibitory.

11. The method of any one of the preceding claims, wherein the MSC reduces the proliferation, quantity, and/or activity of an immune cell, optionally selected from a T cell and NK cell.

12. The method of any one of the preceding claims, wherein the MSC is suitable for in vitro expansion without substantial loss of immunosuppressive properties.

13. The method of any one of the preceding claims, wherein the MSC substantially expresses one or more of CD73, CD90, and CD105.

14. The method of any one of the preceding claims, wherein the MSC substantially does not express one or more of CD14, CD34 and CD45.

15. The method of any one of the preceding claims, wherein the synthetic RNA molecule is mRNA.

16. The method of claim 15, wherein the synthetic RNA molecule is mRNA comprising one or more non-canonical nucleotides selected from 2-thiouridine, 5-azauridine, pseudouridine, 4-thiouridine, 5-methyluridine, 5-methylpseudouridine, 5-aminouridine, 5-aminopseudouridine, 5-hydroxyuridine, 5-hydroxypseudouridine, 5-methoxyuridine, 5-methoxypseudouridine, 5-ethoxyuridine, 5-ethoxypseudouridine, 5-hydroxymethyluridine, 5-hydroxymethylpseudouridine, 5-carboxyuridine, 5-carboxypseudouridine, 5-formyluridine, 5-formylpseudouridine, 5-methyl-5-azauridine, 5-amino-5-azauridine, 5-hydroxy-5-azauridine, 5-methylpseudouridine, 5-aminopseudouridine, 5-hydroxypseudouridine, 4-thio-5-azauridine, 4-thiopseudouridine, 4-thio-5-methyluridine, 4-thio-5-aminouridine, 4-thio-5-hydroxyuridine, 4-thio-5-methyl-5-azauridine, 4-thio-5-amino-5-azauridine, 4-thio-5-hydroxy-5-azauridine, 4-thio-5-methylpseudouridine, 4-thio-5-aminopseudouridine, 4-thio-5-hydroxypseudouridine, 2-thiocytidine, 5-azacytidine, pseudoisocytidine, N4-methylcytidine, N4-aminocytidine, N4-hydroxycytidine, 5-methylcytidine, 5-aminocytidine, 5-hydroxycytidine, 5-methoxycytidine, 5-ethoxycytidine, 5-hydroxymethylcytidine, 5-carboxycytidine, 5-formylcytydine, 5-methyl-5-azacytidine, 5-amino-5-azacytidine, 5-hydroxy-5-azacytidine, 5-methylpseudoisocytidine, 5-aminopseudoisocytidine, 5-hydroxypseudoisocytidine, N4-methyl-5-azacytidine, N4-methylpseudoisocytidine, 2-thio-5-azacytidine, 2-thiopseudoisocytidine, 2-thio-N4-methylcytidine, 2-thio-N4-aminocytidine, 2-thio-N4-hydroxycytidine, 2-thio-5-methylcytidine, 2-thio-5-aminocytidine, 2-thio-5-hydroxycytidine, 2-thio-5-methyl-5-azacytidine, 2-thio-5-amino-5-azacytidine, 2-thio-5-hydroxy-5-azacytidine, 2-thio-5-methylpseudoisocytidine, 2-thio-5-aminopseudoisocytidine, 2-thio-5-hydroxypseudoisocytidine, 2-thio-N4-methyl-5-azacytidine, 2-thio-N4-methylpseudoisocytidine, N4-methyl-5-methylcytidine, N4-methyl-5-aminocytidine, N4-methyl-5-hydroxycytidine, N4-methyl-5-methyl-5-azacytidine, N4-methyl-5-amino-5-azacytidine, N4-methyl-5-hydroxy-5-azacytidine, N4-methyl-5-methylpseudoisocytidine, N4-methyl-5-aminopseudoisocytidine, N4-methyl-5-hydroxypseudoisocytidine, N4-amino-5-azacytidine, N4-aminopseudoisocytidine, N4-amino-5-methylcytidine, N4-amino-5-aminocytidine, N4-amino-5-hydroxycytidine, N4-amino-5-methyl-5-azacytidine, N4-amino-5-amino azacytidine, N4-amino-5-hydroxy-5-azacytidine, N4-amino-5-methylpseudoisocytidine, N4-amino-5-aminopseudoisocytidine, N4-amino-5-hydroxypseudoisocytidine, N4-hydroxy-5-azacytidine, N4-hydroxypseudoisocytidine, N4-hydroxy-5-methylcytidine, N4-hydroxy-5-aminocytidine, N4-hydroxy-5-hydroxycytidine, N4-hydroxy-5-methyl-5-azacytidine, N4-hydroxy-5-amino-5-azacytidine, N4-hydroxy-5-hydroxy-5-azacytidine, N4-hydroxy-5-methylpseudoisocytidine, N4-hydroxy-5-aminopseudoisocytidine, N4-hydroxy-5-hydroxypseudoisocytidine, 2-thio-N4-methyl-5-methylcytidine, 2-thio-N4-methyl-5-aminocytidine, 2-thio-N4-methyl-5-hydroxycytidine, 2-thio-N4-methyl-5-methyl-5-azacytidine, 2-thio-N4-methyl-5-amino-5-azacytidine, 2-thio-N4-methyl-5-hydroxy-5-azacytidine, 2-thio-N4-methyl-5-methylpseudoisocytidine, 2-thio-N4-methyl-5-aminopseudoisocytidine, 2-thio-N4-methyl-5-hydroxypseudoisocytidine, 2-thio-N4-amino-5-azacytidine, 2-thio-N4-aminopseudoisocytidine, 2-thio-N4-amino-5-methylcytidine, 2-thio-N4-amino-5-aminocytidine, 2-thio-N4-amino-5-hydroxycytidine, 2-thio-N4-amino-5-methyl-5-azacytidine, 2-thio-N4-amino-5-amino-5-azacytidine, 2-thio-N4-amino-5-hydroxy-5-azacytidine, 2-thio-N4-amino-5-methylpseudoisocytidine, 2-thio-N4-amino-5-aminopseudoisocytidine, 2-thio-N4-amino-5-hydroxypseudoisocytidine, 2-thio-N4-hydroxy-5-azacytidine, 2-thio-N4-hydroxypseudoisocytidine, 2-thio-N4-hydroxy-5-methylcytidine, N4-hydroxy-5-aminocytidine, 2-thio-N4-hydroxy-5-hydroxycytidine, 2-thio-N4-hydroxy-5-methyl-5-azacytidine, 2-thio-N4-hydroxy-5-amino-5-azacytidine, 2-thio-N4-hydroxy-5-hydroxy-5-azacytidine, 2-thio-N4-hydroxy-5-methylpseudoisocytidine, 2-thio-N4-hydroxy-5-aminopseudoisocytidine, 2-thio-N4-hydroxy-5-hydroxypseudoisocytidine, N6-methyladenosine, N6-aminoadenosine, N6-hydroxyadenosine, 7-deazaadenosine, 8-azaadenosine, N6-methyl-7-deazaadenosine, N6-methyl-8-azaadenosine, 7-deaza-8-azaadenosine, N6-methyl-7-deaza-8-azaadenosine, N6-amino-7-deazaadenosine, N6-amino-8-azaadenosine, N6-amino-7-deaza-8-azaadenosine, N6-hydroxyadenosine, N6-hydroxy-7-deazaadenosine, N6-hydroxy-8-azaadenosine, N6-hydroxy-7-deaza-8-azaadenosine, 6-thioguanosine, 7-deazaguanosine, 8-azaguanosine, 6-thio-7-deazaguanosine, 6-thio-8-azaguanosine, 7-deaza-8-azaguanosine, and 6-thio-7-deaza-8-azaguanosine.

17. The method of any one of the preceding claims, wherein the synthetic RNA molecule is in vitro transcribed.

18. The method of any one of the preceding claims, wherein the reprogramming is non-viral.

19. The method of any one of the preceding claims, wherein the reprogramming factor is one or more of Oct4, Sox2, Klf4, c-Myc, I-Myc, Tert, Nanog, and Lin28.

20. The method of any one of the preceding claims, wherein the composition is suitable for use in the treatment of amyotrophic lateral sclerosis (ALS), spinal cord injury, degenerative disc disease, coronary artery disease, acute myocardial infarction, alcoholic liver cirrhosis, hepatitis C virus (HCV)-induced cirrhosis, multiple sclerosis (MS), osteoarthritis (OA), osteoarthritis of the knee, kidney allograft, critical limb ischemia, ischemic cardiomyopathy, Crohn's disease, idiopathic pulmonary fibrosis, anal fistula, spinal cord injury, systemic lupus erythematosus (SLE), acute respiratory distress syndrome (ARDS), acute graft-versus-host disease (aGvHD), preterm bronchopulmonary dysplasia (BPD), autism nonischemic heart failure, and/or Type 2 diabetes mellitus.

21. The method of any one of the preceding claims, wherein the composition is suitable for use in the treatment of an infectious disease, optionally selected from an infection with a pathogen, optionally selected from a bacterium, virus, fungus, or parasite.

22. The method of claim 21, wherein the pathogen is a virus.

23. The method of claim 22, wherein the virus is:
(a) an influenza virus, optionally selected from Type A, Type B, Type C, and Type D influenza viruses, or
(b) a member of the Coronaviridae family, optionally selected from
(i) a betacoronavirus, optionally selected from Severe acute respiratory syndrome coronavirus 2 (SARS-CoV-2), SARS-CoV, Middle East Respiratory Syndrome-Corona Virus (MERS-CoV), HCoV-HKU1, and HCoV-OC43 or
(ii) an alphacoronavirus, optionally selected from HCoV-NL63 and HCoV-229E.

24. The method of claim 23, wherein the virus is SARS-CoV-2.

25. The method of any one of claims 21-24, wherein the virus is SARS-CoV-2, which has caused COVID-19.

26. The method of claim 25, wherein the COVID-19 is characterized by one or more of fever, cough, shortness of breath, diarrhea, upper respiratory symptoms, lower respiratory symptoms, pneumonia, and respiratory distress.

27. The method of any one of claims 21-26, wherein the therapy prevents or mitigates development of acute respiratory distress syndrome (ARDS) in a patient when administered.

28. The method of any one of claims 21-27, wherein the therapy improves oxygenation in a patient when administered.

29. The method of any one of claims 21-28, wherein the therapy prevents or mitigates a transition from respiratory distress to cytokine imbalance in a patient when administered

30. The method of any one of claims 21-29, wherein the therapy reverses or prevents a cytokine storm in a patient when administered.

31. The method of any one of the preceding claims, wherein the therapy reverses or prevents a cytokine storm in the lungs or systemically in a patient when administered.

32. The method of claim 30 or 31, wherein the cytokine storm is selected from one or more of systemic inflammatory response syndrome, cytokine release syndrome, macrophage activation syndrome, and hemophagocytic lymphohistiocytosis.

33. The method of any one of claim 30-32, wherein the therapy reverses or prevents excessive production of one or more inflammatory cytokines in a patient when administered.

34. The method of claim 33, wherein the inflammatory cytokine is one or more of IL-6, IL-1, IL-1 receptor antagonist (IL-1ra), IL-2ra, IL-10, IL-18, TNFα, interferon-γ, CXCL10, and CCL7.

35. The method of any one of the preceding claims, wherein the composition is formulated for one or more of intrathecal, intra-lesional, intra-coronary, intravenous (IV), intra-articular, intramuscular, and intra-endobronchial administration and administration via intrapancreatic endovascular injection, intra-nucleus pulposus, lumbar puncture, intra-myocardium, transendocardium, intra-fistula tract, intermedullary space, intradural space and leg injection.

36. The method of any one of the preceding claims, wherein the composition is formulated for infusion.

37. The method of any one of the preceding claims, wherein the protein secretion signature is increased secretion of about 5, or about 10, or about 15, or about 20, or about 25, or about 30, or about 35, or all of MIP-1 alpha, SDF-1 alpha, IL-27, LIF, IL-1 beta, IL-2, IL-5, IL-12p70, IL-13, IL-17A, IL-31, G-CSF/CSF-3, IFN-gamma, TNF-alpha, HGF, MCP-1, IL-9, bNGF, MIP-3 alpha, Gro-alpha/KC, IL-1alpha, IL-23, MMP-1, IL-18, M-CSF, IL-21, M-CSF, IL-21, CD40L, IL-22, VEGF-A, BLC, Tweak, ENA-78 (LIX), MCP-3, MIF, and Eotaxin-3.

38. The method of any one of the preceding claims, wherein the protein secretion signature is decreased secretion of IL-6, IL-8, and/or IL-4.

39. The method of any one of the preceding claims, wherein the protein secretion signature is
(a) increased secretion of about 5, or about 10, or about 15, or about 20, or about 25, or about 30, or about 35, or all of MIP-1 alpha, SDF-1 alpha, IL-27, LIF, IL-1 beta, IL-2, IL-5, IL-12p70, IL-13, IL-17A, IL-31, G-CSF/CSF-3, IFN-gamma, TNF-alpha, HGF, MCP-1, IL-9, bNGF, MIP-3 alpha, Gro-alpha/KC, IL-1alpha, IL-23, MMP-1, IL-18, M-CSF, IL-21, M-CSF, IL-21, CD40L, IL-22, VEGF-A, BLC, Tweak, ENA-78 (LIX), MCP-3, MIF, and Eotaxin-3 and
(b) decreased secretion of IL-6, IL-8, and/or IL-4.

40. The method of any one of the preceding claims, wherein:
the protein secretion signature is secretion of one, or two, or three, or four, or five, or six, or all of MIP-1 alpha, G-CSF/CSF-3, M-CSF, BLC, Tweak, ENA-78 (LIX), and MCP-3, or
the protein secretion signature is decreased secretion, relative to the IL-6 secretion by a bone marrow-derived MSC, of about 5, or about 10, or about 15, or about 20, or about 25, or about 30, or all of MIP-1 alpha, IL-27, LIF, IL-1 beta, IL-2, IL-5, IL-12p70, IL-13, IL-17A, IL-31, G-CSF/CSF-3, IFN-gamma, TNF-alpha, HGF, IL-9, bNGF, MIP-3 alpha, Gro-alpha/KC, IL-1alpha, IL-23, IL-18, M-CSF, IL-21, M-CSF, IL-21, CD40L, IL-22, BLC, ENA-78 (LIX), MCP-3, MIF, and Eotaxin-3.

41. A cell produced by the method of any of the preceding claims.

42. A method of treating an inflammatory and/or immunomodulatory disease or disorder, comprising:
(a) obtaining a mesenchymal stem cell (MSC),
the MSC having been obtained from reprogramming an induced pluripotent stem cell (iPSC), the reprogramming comprising contacting the iPSC with one or more synthetic RNA molecules encoding a reprogramming factor and
having a protein secretion signature, the protein secretion signature comprising an increased secretion of one or more proteins selected from MIP-1 alpha, SDF-1 alpha, IL-27, LIF, IL-1 beta, IL-2, IL-5, IL-12p70, IL-13, IL-17A, IL-31, G-CSF/CSF-3, IFN-gamma, TNF-alpha, HGF, MCP-1, IL-9, bNGF, MIP-3 alpha, Gro-alpha/KC, IL-1alpha, IL-23, MMP-1, IL-18, M-CSF, IL-21, M-CSF, IL-21, CD40L, IL-22, VEGF-A, BLC, Tweak, ENA-78 (LIX), MCP-3, MIF, and Eotaxin-3 and/or a decreased secretion of one or more proteins selected from IL-6, IL-8, and IL-4, wherein the increased and/or decreased secretion is relative to a bone marrow-derived MSC; and
(b) administering an effective amount of the MSC substantially having the protein secretion signature for therapy to a patient in need thereof.

43. The method of claim 42, wherein the iPSC is derived from a human.

44. The method of claim 43, wherein the iPSC is derived from a subject who is not intended to receive the therapy.

45. The method of any one of claims 42-44, wherein the iPSC is allogeneic to the patient intended to receive the therapy.

46. The method of any one of claims 42-45, wherein the iPSC is from a master cell bank.

47. The method of any one of claims 42-46, wherein the MSC is characterized by low or reduced inflammation.

48. The method of any one of claims 42-47, wherein the MSC is characterized by low or reduced immunogenicity.

49. The method of any one of claims 42-48, wherein the MSC is self-renewing.

50. The method of any one of claims 42-49, wherein the MSC is multipotent.

51. The method of any one of claims 42-50, wherein the MSC is immune inhibitory.

52. The method of any one of claims 42-51, wherein the MSC reduces the proliferation, quantity, and/or activity of an immune cell, optionally selected from a T cell and NK cell.

53. The method of any one of claims 42-52, wherein the MSC is suitable for in vitro expansion without substantial loss of immunosuppressive properties.

54. The method of any one of claims 42-53, wherein the MSC substantially expresses one or more of CD73, CD90, and CD105.

55. The method of any one of claims 42-54, wherein the MSC substantially does not express one or more of CD14, CD34 and CD45.

56. The method of any one of claims 42-55, wherein the synthetic RNA molecule is mRNA.

57. The method of claim 56, wherein the synthetic RNA molecule is mRNA comprising one or more non-canonical nucleotides selected from 2-thiouridine, 5-azauridine, pseudouridine, 4-thiouridine, 5-methyluridine, 5-methylpseudouridine, 5-aminouridine, 5-aminopseudouridine, 5-hydroxyuridine, 5-hydroxypseudouridine, 5-methoxyuridine, 5-methoxypseudouridine, 5-ethoxyuridine, 5-ethoxypseudouridine, 5-hydroxymethyluridine, 5-hydroxymethylpseudouridine, 5-carboxyuridine, 5-carboxypseudouridine, 5-formyluridine, 5-formylpseudouridine, 5-methyl-5-azauridine, 5-amino azauridine, 5-hydroxy-5-azauridine, 5-methylpseudouridine, 5-aminopseudouridine, 5-hydroxypseudouridine, 4-thio-5-azauridine, 4-thiopseudouridine, 4-thio-5-methyluridine, 4-thio-5-aminouridine, 4-thio-5-hydroxyuridine, 4-thio-5-methyl-5-azauridine, 4-thio-5-amino-5-azauridine, 4-thio-5-hydroxy-5-azauridine, 4-thio-5-methylpseudouridine, 4-thio-5-aminopseudouridine, 4-thio-5-hydroxypseudouridine, 2-thiocytidine, 5-azacytidine, pseudoisocytidine, N4-methylcytidine, N4-aminocytidine, N4-hydroxycytidine, 5-methylcytidine, 5-aminocytidine, 5-hydroxycytidine, 5-methoxycytidine, 5-ethoxycytidine, 5-hydroxymethylcytidine, 5-carboxycytidine, 5-formylcytydine, 5-methyl-5-azacytidine, 5-amino-5-azacytidine, 5-hydroxy-5-azacytidine, 5-methylpseudoisocytidine, 5-aminopseudoisocytidine, 5-hydroxypseudoisocytidine, N4-methyl-5-azacytidine, N4-methylpseudoisocytidine, 2-thio-5-azacytidine, 2-thiopseudoisocytidine, 2-thio-N4-methylcytidine, 2-thio-N4-aminocytidine, 2-thio-N4-hydroxycytidine, 2-thio-5-methylcytidine, 2-thio-5-aminocytidine, 2-thio-5-hydroxycytidine, 2-thio-5-methyl-5-azacytidine, 2-thio-5-amino-5-azacytidine, 2-thio-5-hydroxy-5-azacytidine, 2-thio-5-methylpseudoisocytidine, 2-thio-5-aminopseudoisocytidine, 2-thio-5-hydroxypseudoisocytidine, 2-thio-N4-methyl-5-azacytidine, 2-thio-N4-methylpseudoisocytidine, N4-methyl-5-methylcytidine, N4-methyl-5-aminocytidine, N4-methyl-5-hydroxycytidine, N4-methyl-5-methyl-5-azacytidine, N4-methyl-5-amino-5-azacytidine, N4-methyl-5-hydroxy-5-azacytidine, N4-methyl-5-methylpseudoisocytidine, N4-methyl-5-aminopseudoisocytidine, N4-methyl-5-hydroxypseudoisocytidine, N4-amino-5-azacytidine, N4-aminopseudoisocytidine, N4-amino-5-methylcytidine, N4-amino-5-aminocytidine, N4-amino-5-hydroxycytidine, N4-amino-5-methyl-5-azacytidine, N4-amino-5-amino-5-azacytidine, N4-amino-5-hydroxy-5-azacytidine, N4-amino-5-methylpseudoisocytidine, N4-amino-5-aminopseudoisocytidine, N4-amino-5-hydroxypseudoisocytidine, N4-hydroxy-5-azacytidine, N4-hydroxypseudoisocytidine, N4-hydroxy-5-methylcytidine, N4-hydroxy-5-aminocytidine, N4-hydroxy-5-hydroxycytidine, N4-hydroxy-5-methyl-5-azacytidine, N4-hydroxy-5-amino-5-azacytidine, N4-hydroxy-5-hydroxy-5-azacytidine, N4-hydroxy-5-methylpseudoisocytidine, N4-hydroxy-5-aminopseudoisocytidine, N4-hydroxy-5-hydroxypseudoisocytidine, 2-thio-N4-methyl-5-methylcytidine, 2-thio-N4-methyl-5-aminocytidine, 2-thio-N4-methyl-5-hydroxycytidine, 2-thio-N4-methyl-5-methyl-5-azacytidine, 2-thio-N4-methyl-5-amino-5-azacytidine, 2-thio-N4-methyl-5-hydroxy-5-azacytidine, 2-thio-N4-methyl-5-methylpseudoisocytidine, 2-thio-N4-methyl-5-aminopseudoisocytidine, 2-thio-N4-methyl-5-hydroxypseudoisocytidine, 2-thio-N4-amino-5-azacytidine, 2-thio-N4-aminopseudoisocytidine, 2-thio-N4-amino-5-methylcytidine, 2-thio-N4-amino-5-aminocytidine, 2-thio-N4-amino-5-hydroxycytidine, 2-thio-N4-amino-5-methyl-5-azacytidine, 2-thio-N4-amino-5-amino-5-azacytidine, 2-thio-N4-amino-5-hydroxy-5-azacytidine, 2-thio-N4-amino-5-methylpseudoisocytidine, 2-thio-N4-amino-5-aminopseudoisocytidine, 2-thio-N4-amino-5-hydroxypseudoisocytidine, 2-thio-N4-hydroxy azacytidine, 2-thio-N4-hydroxypseudoisocytidine, 2-thio-N4-hydroxy-5-methylcytidine, N4-hydroxy aminocytidine, 2-thio-N4-hydroxy-5-hydroxycytidine, 2-thio-N4-hydroxy-5-methyl-5-azacytidine, 2-thio-N4-hydroxy-5-amino-5-azacytidine, 2-thio-N4-hydroxy-5-hydroxy-5-azacytidine, 2-thio-N4-hydroxy-5-methylpseudoisocytidine, 2-thio-N4-hydroxy-5-aminopseudoisocytidine, 2-thio-N4-hydroxy-5-hydroxypseudoisocytidine, N6-methyladenosine, N6-aminoadenosine, N6-hydroxyadenosine, 7-deazaadenosine, 8-azaadenosine, N6-methyl-7-deazaadenosine, N6-methyl-8-azaadenosine, 7-deaza-8-azaadenosine, N6-methyl-7-deaza-8-azaadenosine, N6-amino-7-deazaadenosine, N6-amino azaadenosine, N6-amino-7-deaza-8-azaadenosine, N6-hydroxyadenosine, N6-hydroxy-7-deazaadenosine, N6-hydroxy-8-azaadenosine, N6-hydroxy-7-deaza-8-azaadenosine, 6-thioguanosine, 7-deazaguanosine, 8-azaguanosine, 6-thio-7-deazaguanosine, 6-thio-8-azaguanosine, 7-deaza-8-azaguanosine, and 6-thio-7-deaza-8-azaguanosine.

58. The method of any one of claims 42-57, wherein the synthetic RNA molecule is in vitro transcribed.

59. The method of any one of claims 42-58, wherein the reprogramming is non-viral.

60. The method of any one of claims 42-59, wherein the reprogramming factor is one or more of Oct4, Sox2, Klf4, c-Myc, I-Myc, Tert, Nanog, and Lin28.

61. The method of any one of claims 42-60, wherein the composition is suitable for use in the treatment of amyotrophic lateral sclerosis (ALS), spinal cord injury, degenerative disc disease, coronary artery disease, acute myocardial infarction, alcoholic liver cirrhosis, hepatitis C virus (HCV)-induced cirrhosis, multiple sclerosis (MS), osteoarthritis (OA), osteoarthritis of the knee, kidney allograft, critical limb ischemia, ischemic cardiomyopathy, Crohn's disease, idiopathic pulmonary fibrosis, anal fistula, spinal cord injury, systemic lupus erythematosus (SLE), acute respiratory distress syndrome (ARDS), acute graft-versus-host disease (aGvHD), preterm bronchopulmonary dysplasia (BPD), autism nonischemic heart failure, and/or Type 2 diabetes mellitus.

62. The method of any one of claims 42-61, wherein the composition is suitable for use in the treatment of an infectious disease, optionally selected from an infection with a pathogen, optionally selected from a bacterium, virus, fungus, or parasite.

63. The method of claim 62, wherein the pathogen is a virus.

64. The method of claim 63, wherein the virus is:
(a) an influenza virus, optionally selected from Type A, Type B, Type C, and Type D influenza viruses, or
(b) a member of the Coronaviridae family, optionally selected from
(i) a betacoronavirus, optionally selected from Severe acute respiratory syndrome coronavirus 2 (SARS-CoV-2), SARS-CoV, Middle East Respiratory Syndrome-Corona Virus (MERS-CoV), HCoV-HKU1, and HCoV-OC43 or
(ii) an alphacoronavirus, optionally selected from HCoV-NL63 and HCoV-229E.

65. The method of claim 64, wherein the virus is SARS-CoV-2.

66. The method of any one of claims 62-65, wherein the virus is SARS-CoV-2, which has caused COVID-19.

67. The method of claim 66, wherein the COVID-19 is characterized by one or more of fever, cough, shortness of breath, diarrhea, upper respiratory symptoms, lower respiratory symptoms, pneumonia, and respiratory distress.

68. The method of any one of claims to 62-67, wherein the therapy prevents or mitigates development of acute respiratory distress syndrome (ARDS) in the patient when administered.

69. The method of any one of claims to 62-68, wherein the therapy improves oxygenation in the patient when administered.

70. The method of any one of claims to 62-69, wherein the therapy prevents or mitigates a transition from respiratory distress to cytokine imbalance in the patient when administered.

71. The method of any one of claims 62-70, wherein the therapy reverses or prevents a cytokine storm in the patient when administered.

72. The method of any one of claims 42-71, wherein the therapy reverses or prevents a cytokine storm in the lungs or systemically in the patient when administered.

73. The method of claim 71 or 72, wherein the cytokine storm is selected from one or more of systemic inflammatory response syndrome, cytokine release syndrome, macrophage activation syndrome, and hemophagocytic lymphohistiocytosis.

74. The method of any one of claims 71-73, wherein the therapy reverses or prevents excessive production of one or more inflammatory cytokines in the patient when administered.

75. The method of claim 74, wherein the inflammatory cytokine is one or more of IL-6, IL-1, IL-1 receptor antagonist (IL-1ra), IL-2ra, IL-10, IL-18, TNFα, interferon-γ, CXCL10, and CCL7.

76. The method of any one of claims 42-75, wherein the composition is formulated for one or more of intrathecal, intra-lesional, intra-coronary, intravenous (IV), intra-articular, intramuscular, and intra-endobronchial administration and administration via intrapancreatic endovascular injection, intra-nucleus pulposus, lumbar puncture, intra-myocardium, transendocardium, intra-fistula tract, intermedullary space, intradural space and leg injection.

77. The method of any one of claims 42-76, wherein the composition is formulated for infusion.

78. The method of any one of claims 42-77, wherein the protein secretion signature is increased secretion of about 5, or about 10, or about 15, or about 20, or about 25, or about 30, or about 35, or all of MIP-1 alpha, SDF-1 alpha, IL-27, LIF, IL-1 beta, IL-2, IL-5, IL-12p70, IL-13, IL-17A, IL-31, G-CSF/CSF-3, IFN-gamma, TNF-alpha, HGF, MCP-1, IL-9, bNGF, MIP-3 alpha, Gro-alpha/KC, IL-1alpha, IL-23, MMP-1, IL-18, M-CSF, IL-21, M-CSF, IL-21, CD40L, IL-22, VEGF-A, BLC, Tweak, ENA-78 (LIX), MCP-3, MIF, and Eotaxin-3.

79. The method of any one of claims 42-78, wherein the protein secretion signature is decreased secretion of IL-6, IL-8, and/or IL-4.

80. The method of any one of claims 42-79, wherein the protein secretion signature is
(a) increased secretion of about 5, or about 10, or about 15, or about 20, or about 25, or about 30, or about 35, or all of MIP-1 alpha, SDF-1 alpha, IL-27, LIF, IL-1 beta, IL-2, IL-5, IL-12p70, IL-13, IL-17A, IL-31, G-CSF/CSF-3, IFN-gamma, TNF-alpha, HGF, MCP-1, IL-9, bNGF, MIP-3 alpha, Gro-alpha/KC, IL-1alpha, IL-23, MMP-1, IL-18, M-CSF, IL-21, M-CSF, IL-21, CD40L, IL-22, VEGF-A, BLC, Tweak, ENA-78 (LIX), MCP-3, MIF, and Eotaxin-3 and
(b) decreased secretion of IL-6, IL-8, and/or IL-4.

81. The method of any one of claims 42-80, wherein the protein secretion signature is secretion of one, or two, or three, or four, or five, or six, or all of MIP-1 alpha, G-CSF/CSF-3, M-CSF, BLC, Tweak, ENA-78 (LIX), and MCP-3.

82. The method of any one of claims 42-81, wherein the protein secretion signature is decreased secretion, relative to the IL-6 secretion by a bone marrow-derived MSC, of about 5, or about 10, or about 15, or about 20, or about 25, or about 30, or all of MIP-1 alpha, IL-27, LIF, IL-1 beta, IL-2, IL-5, IL-12p70, IL-13, IL-17A, IL-31, G-CSF/CSF-3, IFN-gamma, TNF-alpha, HGF, IL-9, bNGF, MIP-3 alpha, Gro-alpha/KC, IL-1alpha, IL-23, IL-18, M-CSF, IL-21, M-CSF, IL-21, CD40L, IL-22, BLC, ENA-78 (LIX), MCP-3, MIF, and Eotaxin-3.

83. A method of treating or mitigating a respiratory distress associated with an infection, comprising:
(a) obtaining a mesenchymal stem cell (MSC),
the MSC having been obtained from reprogramming an induced pluripotent stem cell (iPSC), the reprogramming comprising contacting the iPSC with one or more synthetic RNA molecules encoding a reprogramming factor and
having a protein secretion signature, the protein secretion signature comprising an increased secretion of one or more proteins selected from MIP-1 alpha, SDF-1 alpha, IL-27, LIF, IL-1 beta, IL-2, IL-5, IL-12p70, IL-13, IL-17A, IL-31, G-CSF/CSF-3, IFN-gamma, TNF-alpha, HGF, MCP-1, IL-9, bNGF, MIP-3 alpha, Gro-alpha/KC, IL-1alpha, IL-23, MMP-1, IL-18, M-CSF, IL-21, M-CSF, IL-21, CD40L, IL-22, VEGF-A, BLC, Tweak, ENA-78 (LIX), MCP-3, MIF, and Eotaxin-3 and/or a decreased secretion of one or more proteins selected from IL-6, IL-8, and IL-4, wherein the increased and/or decreased secretion is relative to a bone marrow-derived MSC; and
(b) administering an effective amount of the MSC substantially having the protein secretion signature for therapy to a patient in need thereof.

84. The method of claim 83, wherein the iPSC is derived from a human.

85. The method of claim 84, wherein the iPSC is derived from a subject who is not intended to receive the therapy.

86. The method of any one of claims 83-85, wherein the iPSC is allogeneic to the patient intended to receive the therapy.

87. The method of any one of claims 83-86, wherein the iPSC is from a master cell bank.

88. The method of any one of claims 83-87, wherein the MSC is characterized by low or reduced inflammation.

89. The method of any one of claims 83-88, wherein the MSC is characterized by low or reduced immunogenicity.

90. The method of any one of claims 83-89, wherein the MSC is self-renewing.

91. The method of any one of claims 83-90, wherein the MSC is multipotent.

92. The method of any one of claims 83-91, wherein the MSC is immune inhibitory.

93. The method of any one of claims 83-92, wherein the MSC reduces the proliferation, quantity, and/or activity of an immune cell, optionally selected from a T cell and NK cell.

94. The method of any one of claims 83-93, wherein the MSC is suitable for in vitro expansion without substantial loss of immunosuppressive properties.

95. The method of any one of claims 83-94, wherein the MSC substantially expresses one or more of CD73, CD90, and CD105.

96. The method of any one of claims 83-95, wherein the MSC substantially does not express one or more of CD14, CD34 and CD45.

97. The method of any one of claims 83-96, wherein the synthetic RNA molecule is mRNA.

98. The method of claim 97, wherein the synthetic RNA molecule is mRNA comprising one or more non-canonical nucleotides selected from 2-thiouridine, 5-azauridine, pseudouridine, 4-thiouridine, 5-methyluridine, 5-methylpseudouridine, 5-aminouridine, 5-aminopseudouridine, 5-hydroxyuridine, 5-hydroxypseudouridine, 5-methoxyuridine, 5-methoxypseudouridine, 5-ethoxyuridine, 5-ethoxypseudouridine, 5-hydroxymethyluridine, 5-hydroxymethylpseudouridine, 5-carboxyuridine, 5-carboxypseudouridine, 5-formyluridine, 5-formylpseudouridine, 5-methyl-5-azauridine, 5-amino-5-azauridine, 5-hydroxy-5-azauridine, 5-methylpseudouridine, 5-aminopseudouridine, 5-hydroxypseudouridine, 4-thio-5-azauridine, 4-thiopseudouridine, 4-thio-5-methyluridine, 4-thio-5-aminouridine, 4-thio-5-hydroxyuridine, 4-thio-5-methyl-5-azauridine, 4-thio-5-amino-5-azauridine, 4-thio-5-hydroxy-5-azauridine, 4-thio-5-methylpseudouridine, 4-thio-5-aminopseudouridine, 4-thio-5-hydroxypseudouridine, 2-thiocytidine, 5-azacytidine, pseudoisocytidine, N4-methylcytidine, N4-aminocytidine, N4-hydroxycytidine, 5-methylcytidine, 5-aminocytidine, 5-hydroxycytidine, 5-methoxycytidine, 5-ethoxycytidine, 5-hydroxymethylcytidine, 5-carboxycytidine, 5-formylcytydine, 5-methyl-5-azacytidine, 5-amino-5-azacytidine, 5-hydroxy-5-azacytidine, 5-methylpseudoisocytidine, 5-aminopseudoisocytidine, 5-hydroxypseudoisocytidine, N4-methyl-5-azacytidine, N4-methylpseudoisocytidine, 2-thio-5-azacytidine, 2-thiopseudoisocytidine, 2-thio-N4-methylcytidine, 2-thio-N4-aminocytidine, 2-thio-N4-hydroxycytidine, 2-thio-5-methylcytidine, 2-thio-5-aminocytidine, 2-thio-5-hydroxycytidine, 2-thio-5-methyl-5-azacytidine, 2-thio-5-amino-5-azacytidine, 2-thio-5-hydroxy-5-azacytidine, 2-thio-5-methylpseudoisocytidine, 2-thio-5-aminopseudoisocytidine, 2-thio-5-hydroxypseudoisocytidine, 2-thio-N4-methyl-5-azacytidine, 2-thio-N4-methylpseudoisocytidine, N4-methyl-5-methylcytidine, N4-methyl-5-aminocytidine, N4-methyl-5-hydroxycytidine, N4-methyl-5-methyl-5-azacytidine, N4-methyl-5-amino-5-azacytidine, N4-methyl-5-hydroxy-5-azacytidine, N4-methyl-5-methylpseudoisocytidine, N4-methyl-5-aminopseudoisocytidine, N4-methyl-5-hydroxypseudoisocytidine, N4-amino-5-azacytidine, N4-aminopseudoisocytidine, N4-amino-5-methylcytidine, N4-amino-5-aminocytidine, N4-amino-5-hydroxycytidine, N4-amino-5-methyl-5-azacytidine, N4-amino-5-amino-5-azacytidine, N4-amino-5-hydroxy-5-azacytidine, N4-amino-5-methylpseudoisocytidine, N4-amino-5-aminopseudoisocytidine, N4-amino-5-hydroxypseudoisocytidine, N4-hydroxy-5-azacytidine, N4-hydroxypseudoisocytidine, N4-hydroxy-5-methylcytidine, N4-hydroxy-5-aminocytidine, N4-hydroxy-5-hydroxycytidine, N4-hydroxy-5-methyl-5-azacytidine, N4-hydroxy-5-amino-5-azacytidine, N4-hydroxy-5-hydroxy-5-azacytidine, N4-hydroxy-5-methylpseudoisocytidine, N4-hydroxy-5-aminopseudoisocytidine, N4-hydroxy-5-hydroxypseudoisocytidine, 2-thio-N4-methyl-5-methylcytidine, 2-thio-N4-methyl-5-aminocytidine, 2-thio-N4-methyl-5-hydroxycytidine, 2-thio-N4-methyl-5-methyl-5-azacytidine, 2-thio-N4-methyl-5-amino-5-azacytidine, 2-thio-N4-methyl-5-hydroxy-5-azacytidine, 2-thio-N4-methyl-5-methylpseudoisocytidine, 2-thio-N4-methyl-5-aminopseudoisocytidine, 2-thio-N4-methyl-5-hydroxypseudoisocytidine, 2-thio-N4-amino-5-azacytidine, 2-thio-N4-aminopseudoisocytidine, 2-thio-N4-amino-5-methylcytidine, 2-thio-N4-amino-5-aminocytidine, 2-thio-N4-amino-5-hydroxycytidine, 2-thio-N4-amino-5-methyl-5-azacytidine, 2-thio-N4-amino-5-amino-5-azacytidine, 2-thio-N4-amino-5-hydroxy-5-azacytidine, 2-thio-N4-amino-5-methylpseudoisocytidine, 2-thio-N4-amino-5-aminopseudoisocytidine, 2-thio-N4-amino-5-hydroxypseudoisocytidine, 2-thio-N4-hydroxy-5-azacytidine, 2-thio-N4-hydroxypseudoisocytidine, 2-thio-N4-hydroxy-5-methylcytidine, N4-hydroxy-5-aminocytidine, 2-thio-N4-hydroxy-5-hydroxycytidine, 2-thio-N4-hydroxy-5-methyl-5-azacytidine, 2-thio-N4-hydroxy-5-amino-5-azacytidine, 2-thio-N4-hydroxy-5-hydroxy-5-azacytidine, 2-thio-N4-hydroxy-5-methylpseudoisocytidine, 2-thio-N4-hydroxy-5-aminopseudoisocytidine, 2-thio-N4-hydroxy-5-hydroxypseudoisocytidine, N6-methyladenosine, N6-aminoadenosine, N6-hydroxyadenosine, 7-deazaadenosine, 8-azaadenosine, N6-methyl-7-deazaadenosine, N6-methyl-8-azaadenosine, 7-deaza-8-azaadenosine, N6-methyl-7-deaza-8-azaadenosine, N6-amino-7-deazaadenosine, N6-amino-8-azaadenosine, N6-amino-7-deaza-8-azaadenosine, N6-hydroxyadenosine, N6-hydroxy-7-deazaadenosine, N6-hydroxy-8-azaadenosine, N6-hydroxy-7-deaza-8-azaadenosine, 6-thioguanosine, 7-deazaguanosine, 8-azaguanosine, 6-thio-7-deazaguanosine, 6-thio-8-azaguanosine, 7-deaza-8-azaguanosine, and 6-thio-7-deaza-8-azaguanosine.

99. The method of any one of claims 83-98, wherein the synthetic RNA molecule is in vitro transcribed.

100. The method of any one of claims 83-99, wherein the reprogramming is non-viral.

101. The method of any one of claims 83-100, wherein the reprogramming factor is one or more of Oct4, Sox2, Klf4, c-Myc, I-Myc, Tert, Nanog, and Lin28.

102. The method of any one of claims 83-101, wherein the composition is suitable for use in the treatment of amyotrophic lateral sclerosis (ALS), spinal cord injury, degenerative disc disease, coronary artery disease, acute myocardial infarction, alcoholic liver cirrhosis, hepatitis C virus (HCV)-induced cirrhosis, multiple sclerosis (MS), osteoarthritis (OA), osteoarthritis of the knee, kidney allograft, critical limb ischemia, ischemic cardiomyopathy, Crohn's disease, idiopathic pulmonary fibrosis, anal fistula, spinal cord injury, systemic lupus erythematosus (SLE), acute respiratory distress syndrome (ARDS), acute graft-versus-host disease (aGvHD), preterm bronchopulmonary dysplasia (BPD), autism nonischemic heart failure, and/or Type 2 diabetes mellitus.

103. The method of any one of claims 83-102, wherein the composition is suitable for use in the treatment of an infectious disease, optionally selected from an infection with a pathogen, optionally selected from a bacterium, virus, fungus, or parasite.

104. The method of claim 103, wherein the pathogen is a virus.

105. The method of claim 104, wherein the virus is:
(a) an influenza virus, optionally selected from Type A, Type B, Type C, and Type D influenza viruses, or
(b) a member of the Coronaviridae family, optionally selected from
(i) a betacoronavirus, optionally selected from Severe acute respiratory syndrome coronavirus 2 (SARS-CoV-2), SARS-CoV, Middle East Respiratory Syndrome-Corona Virus (MERS-CoV), HCoV-HKU1, and HCoV-OC43 or
(ii) an alphacoronavirus, optionally selected from HCoV-NL63 and HCoV-229E.

106. The method of claim 105, wherein the virus is SARS-CoV-2.

107. The method of any one of claims 103-106, wherein the virus is SARS-CoV-2, which has caused COVID-19.

108. The method of claim 107, wherein the COVID-19 is characterized by the respiratory distress and one or more of fever, cough, shortness of breath, diarrhea, upper respiratory symptoms, lower respiratory symptoms, and pneumonia.

109. The method of any one of claims 103-108, wherein the therapy prevents or mitigates development of acute respiratory distress syndrome (ARDS) in the patient when administered.

110. The method of any one of claims 103-109, wherein the therapy improves oxygenation in the patient when administered.

111. The method of any one of claims 103-110, wherein the therapy prevents or mitigates a transition from respiratory distress to cytokine imbalance in the patient when administered

112. The method of any one of claims 103-111, wherein the therapy reverses or prevents a cytokine storm in the patient when administered.

113. The method of any one of claims 83-112, wherein the therapy reverses or prevents a cytokine storm in the lungs or systemically in the patient when administered.

114. The method of claim 112 or 113, wherein the cytokine storm is selected from one or more of systemic inflammatory response syndrome, cytokine release syndrome, macrophage activation syndrome, and hemophagocytic lymphohistiocytosis.

115. The method of any one of claims 112-114, wherein the therapy reverses or prevents excessive production of one or more inflammatory cytokines in the patient when administered.

116. The method of claim 115, wherein the inflammatory cytokine is one or more of IL-6, IL-1, IL-1 receptor antagonist (IL-1ra), IL-2ra, IL-10, IL-18, TNFα, interferon-γ, CXCL10, and CCL7.

117. The method of any one of claims 83-116, wherein the composition is formulated for one or more of intrathecal, intra-lesional, intra-coronary, intravenous (IV), intra-articular, intramuscular, and intra-endobronchial administration and administration via intrapancreatic endovascular injection, intra-nucleus pulposus, lumbar puncture, intra-myocardium, transendocardium, intra-fistula tract, intermedullary space, intradural space and leg injection.

118. The method of any one of claims 83-117, wherein the composition is formulated for infusion.

119. The method of any one of claims 83-118, wherein the protein secretion signature is increased secretion of about 5, or about 10, or about 15, or about 20, or about 25, or about 30, or about 35, or all of MIP-1 alpha, SDF-1 alpha, IL-27, LIF, IL-1 beta, IL-2, IL-5, IL-12p70, IL-13, IL-17A, IL-31, G-CSF/CSF-3, IFN-gamma, TNF-alpha, HGF, MCP-1, IL-9, bNGF, MIP-3 alpha, Gro-alpha/KC, IL-1alpha, IL-23, MMP-1, IL-18, M-CSF, IL-21, M-CSF, IL-21, CD40L, IL-22, VEGF-A, BLC, Tweak, ENA-78 (LIX), MCP-3, MIF, and Eotaxin-3.

120. The method of any one of claims 83-119, wherein the protein secretion signature is decreased secretion of IL-6, IL-8, and/or IL-4.

121. The method of any one of claims 83-120, wherein the protein secretion signature is
(a) increased secretion of about 5, or about 10, or about 15, or about 20, or about 25, or about 30, or about 35, or all of MIP-1 alpha, SDF-1 alpha, IL-27, LIF, IL-1 beta, IL-2, IL-5, IL-12p70, IL-13, IL-17A, IL-31, G-CSF/CSF-3, IFN-gamma, TNF-alpha, HGF, MCP-1, IL-9, bNGF, MIP-3 alpha, Gro-alpha/KC, IL-1alpha, IL-23, MMP-1, IL-18, M-CSF, IL-21, M-CSF, IL-21, CD40L, IL-22, VEGF-A, BLC, Tweak, ENA-78 (LIX), MCP-3, MIF, and Eotaxin-3 and
(b) decreased secretion of IL-6, IL-8, and/or IL-4.

122. The method of any one of claims 83-121, wherein the protein secretion signature is secretion of one, or two, or three, or four, or five, or six, or all of MIP-1 alpha, G-CSF/CSF-3, M-CSF, BLC, Tweak, ENA-78 (LIX), and MCP-3.

123. The method of any one of claims 83-122, wherein the protein secretion signature is decreased secretion, relative to the IL-6 secretion by a bone marrow-derived MSC, of about 5, or about 10, or about 15, or about 20, or about 25, or about 30, or all of MIP-1 alpha, IL-27, LIF, IL-1 beta, IL-2, IL-5, IL-12p70, IL-13, IL-17A, IL-31, G-CSF/CSF-3, IFN-gamma, TNF-alpha, HGF, IL-9, bNGF, MIP-3 alpha, Gro-alpha/KC, IL-1alpha, IL-23, IL-18, M-CSF, IL-21, M-CSF, IL-21, CD40L, IL-22, BLC, ENA-78 (LIX), MCP-3, MIF, and Eotaxin-3.

124. A method of treating acute respiratory distress syndrome (ARDS) associated with a SARS-CoV-2 infection, comprising:
(a) obtaining a mesenchymal stem cell (MSC),
the MSC having been obtained from reprogramming an induced pluripotent stem cell (iPSC), the reprogramming comprising contacting the iPSC with one or more synthetic RNA molecules encoding a reprogramming factor and
having a protein secretion signature, the protein secretion signature comprising an increased secretion of one or more proteins selected from MIP-1 alpha, SDF-1 alpha, IL-27, LIF, IL-1 beta, IL-2, IL-5, IL-12p70, IL-13, IL-17A, IL-31, G-CSF/CSF-3, IFN-gamma, TNF-alpha, HGF, MCP-1, IL-9, bNGF, MIP-3 alpha, Gro-alpha/KC, IL-1alpha, IL-23, MMP-1, IL-18, M-CSF, IL-21, M-CSF, IL-21, CD40L, IL-22, VEGF-A, BLC, Tweak, ENA-78 (LIX), MCP-3, MIF, and Eotaxin-3 and/or a decreased secretion of one or more proteins selected from IL-6, IL-8, and IL-4, wherein the increased and/or decreased secretion is relative to a bone marrow-derived MSC; and
(b) administering an effective amount of the MSC substantially having the protein secretion signature for therapy to a patient in need thereof.

125. The method of claim 124, wherein the iPSC is derived from a human.

126. The method of claim 125, wherein the iPSC is derived from a subject who is not intended to receive the therapy.

127. The method of any one of claims 124-126, wherein the iPSC is allogeneic to the patient intended to receive the therapy.

128. The method of any one of claims 124-127, wherein the iPSC is from a master cell bank.

129. The method of any one of claims 124-128, wherein the MSC is characterized by low or reduced inflammation.

130. The method of any one of claims 124-129, wherein the MSC is characterized by low or reduced immunogenicity.

131. The method of any one of claims 124-130, wherein the MSC is self-renewing.

132. The method of any one of claims 124-131, wherein the MSC is multipotent.

133. The method of any one of claims 124-132, wherein the MSC is immune inhibitory.

134. The method of any one of claims 124-133, wherein the MSC reduces the proliferation, quantity, and/or activity of an immune cell, optionally selected from a T cell and NK cell.

135. The method of any one of claims 124-134, wherein the MSC is suitable for in vitro expansion without substantial loss of immunosuppressive properties.

136. The method of any one of claims 124-135, wherein the MSC substantially expresses one or more of CD73, CD90, and CD105.

137. The method of any one of claims 124-136, wherein the MSC substantially does not express one or more of CD14, CD34 and CD45.

138. The method of any one of claims 124-137, wherein the synthetic RNA molecule is mRNA.

139. The method of claim 138, wherein the synthetic RNA molecule is mRNA comprising one or more non-canonical nucleotides selected from 2-thiouridine, 5-azauridine, pseudouridine, 4-thiouridine, 5-methyluridine, 5-methylpseudouridine, 5-aminouridine, 5-aminopseudouridine, 5-hydroxyuridine, 5-hydroxypseudouridine, 5-methoxyuridine, 5-methoxypseudouridine, 5-ethoxyuridine, 5-ethoxypseudouridine, 5-hydroxymethyluridine, 5-hydroxymethylpseudouridine, 5-carboxyuridine, 5-carboxypseudouridine, 5-formyluridine, 5-formylpseudouridine, 5-methyl-5-azauridine, 5-amino-5-azauridine, 5-hydroxy-5-azauridine, 5-methylpseudouridine, 5-aminopseudouridine, 5-hydroxypseudouridine, 4-thio-5-azauridine, 4-thiopseudouridine, 4-thio-5-methyluridine, 4-thio-5-aminouridine, 4-thio-5-hydroxyuridine, 4-thio-5-methyl-5-azauridine, 4-thio-5-amino-5-azauridine, 4-thio-5-hydroxy-5-azauridine, 4-thio-5-methylpseudouridine, 4-thio-5-aminopseudouridine, 4-thio-5-hydroxypseudouridine, 2-thiocytidine, 5-azacytidine, pseudoisocytidine, N4-methylcytidine, N4-aminocytidine, N4-hydroxycytidine, 5-methylcytidine, 5-aminocytidine, 5-hydroxycytidine, 5-methoxycytidine, 5-ethoxycytidine, 5-hydroxymethylcytidine, 5-carboxycytidine, 5-formylcytydine, 5-methyl-5-azacytidine, 5-amino-5-azacytidine, 5-hydroxy-5-azacytidine, 5-methylpseudoisocytidine, 5-aminopseudoisocytidine, 5-hydroxypseudoisocytidine, N4-methyl-5-azacytidine, N4-methylpseudoisocytidine, 2-thio-5-azacytidine, 2-thiopseudoisocytidine, 2-thio-N4-methylcytidine, 2-thio-N4-aminocytidine, 2-thio-N4-hydroxycytidine, 2-thio-5-methylcytidine, 2-thio-5-aminocytidine, 2-thio-5-hydroxycytidine, 2-thio-5-methyl-5-azacytidine, 2-thio-5-amino-5-azacytidine, 2-thio-5-hydroxy-5-azacytidine, 2-thio-5-methylpseudoisocytidine, 2-thio-5-aminopseudoisocytidine, 2-thio-5-hydroxypseudoisocytidine, 2-thio-N4-methyl-5-azacytidine, 2-thio-N4-methylpseudoisocytidine, N4-methyl-5-methylcytidine, N4-methyl-5-aminocytidine, N4-methyl-5-hydroxycytidine, N4-methyl-5-methyl-5-azacytidine, N4-methyl-5-amino-5-azacytidine, N4-methyl-5-hydroxy-5-azacytidine, N4-methyl-5-methylpseudoisocytidine, N4-methyl-5-aminopseudoisocytidine, N4-methyl-5-hydroxypseudoisocytidine, N4-amino-5-azacytidine, N4-aminopseudoisocytidine, N4-amino-5-methylcytidine, N4-amino-5-aminocytidine, N4-amino-5-hydroxycytidine, N4-amino-5-methyl-5-azacytidine, N4-amino-5-amino-5-azacytidine, N4-amino-5-hydroxy-5-azacytidine, N4-amino-5-methylpseudoisocytidine, N4-amino-5-aminopseudoisocytidine, N4-amino-5-hydroxypseudoisocytidine, N4-hydroxy-5-azacytidine, N4-hydroxypseudoisocytidine, N4-hydroxy-5-methylcytidine, N4-hydroxy-5-aminocytidine, N4-hydroxy-5-hydroxycytidine, N4-hydroxy-5-methyl-5-azacytidine, N4-hydroxy-5-amino-5-azacytidine, N4-hydroxy-5-hydroxy-5-azacytidine, N4-hydroxy-5-methylpseudoisocytidine, N4-hydroxy-5-aminopseudoisocytidine, N4-hydroxy-5-hydroxypseudoisocytidine, 2-thio-N4-methyl-5-methylcytidine, 2-thio-N4-methyl-5-aminocytidine, 2-thio-N4-methyl-5-hydroxycytidine, 2-thio-N4-methyl-5-methyl-5-azacytidine, 2-thio-N4-methyl-5-amino-5-azacytidine, 2-thio-N4-methyl-5-hydroxy-5-azacytidine, 2-thio-N4-methyl-5-methylpseudoisocytidine, 2-thio-N4-methyl-5-aminopseudoisocytidine, 2-thio-N4-methyl-5-hydroxypseudoisocytidine, 2-thio-N4-amino-5-azacytidine, 2-thio-N4-aminopseudoisocytidine, 2-thio-N4-amino-5-methylcytidine, 2-thio-N4-amino-5-aminocytidine, 2-thio-N4-amino-5-hydroxycytidine, 2-thio-N4-amino-5-methyl-5-azacytidine, 2-thio-N4-amino-5-amino-5-azacytidine, 2-thio-N4-amino-5-hydroxy-5-azacytidine, 2-thio-N4-amino-5-methylpseudoisocytidine, 2-thio-N4-amino-5-aminopseudoisocytidine, 2-thio-N4-amino-5-hydroxypseudoisocytidine, 2-thio-N4-hydroxy-5-azacytidine, 2-thio-N4-hydroxypseudoisocytidine, 2-thio-N4-hydroxy-5-methylcytidine, N4-hydroxy-5-aminocytidine, 2-thio-N4-hydroxy-5-hydroxycytidine, 2-thio-N4-hydroxy-5-methyl-5-azacytidine, 2-thio-N4-hydroxy-5-amino-5-azacytidine, 2-thio-N4-hydroxy-5-hydroxy-5-azacytidine, 2-thio-N4-hydroxy-5-methylpseudoisocytidine, 2-thio-N4-hydroxy-5-aminopseudoisocytidine, 2-thio-N4-hydroxy-5-hydroxypseudoisocytidine, N6-methyladenosine, N6-aminoadenosine, N6-hydroxyadenosine, 7-deazaadenosine, 8-azaadenosine, N6-methyl-7-deazaadenosine, N6-methyl-8-azaadenosine, 7-deaza-8-azaadenosine, N6-methyl-7-deaza-8-azaadenosine, N6-amino-7-deazaadenosine, N6-amino-8-azaadenosine, N6-amino-7-deaza-8-azaadenosine, N6-hydroxyadenosine, N6-hydroxy-7-deazaadenosine, N6-hydroxy-8-azaadenosine, N6-hydroxy-7-deaza-8-azaadenosine, 6-thioguanosine, 7-deazaguanosine, 8-azaguanosine, 6-thio-7-deazaguanosine, 6-thio-8-azaguanosine, 7-deaza-8-azaguanosine, and 6-thio-7-deaza-8-azaguanosine.

140. The method of any one of claims 124-139, wherein the synthetic RNA molecule is in vitro transcribed.

141. The method of any one of claims 124-140, wherein the reprogramming is non-viral.

142. The method of any one of claims 124-141, wherein the reprogramming factor is one or more of Oct4, Sox2, Klf4, c-Myc, I-Myc, Tert, Nanog, and Lin28.

143. The method of any one of claims 124-142, wherein the composition is suitable for use in the treatment of amyotrophic lateral sclerosis (ALS), spinal cord injury, degenerative disc disease, coronary artery disease, acute myocardial infarction, alcoholic liver cirrhosis, hepatitis C virus (HCV)-induced cirrhosis, multiple sclerosis (MS), osteoarthritis (OA), osteoarthritis of the knee, kidney allograft, critical limb ischemia, ischemic cardiomyopathy, Crohn's disease, idiopathic pulmonary fibrosis, anal fistula, spinal cord injury, systemic lupus erythematosus (SLE), acute respiratory distress syndrome (ARDS), acute graft-versus-host disease (aGvHD), preterm bronchopulmonary dysplasia (BPD), autism nonischemic heart failure, and/or Type 2 diabetes mellitus.

144. The method of any one of claims 124-143, wherein the composition is suitable for use in the treatment of an infectious disease, optionally selected from an infection with a pathogen, optionally selected from a bacterium, virus, fungus, or parasite.

145. The method of claim 144, wherein the pathogen is a virus.

146. The method of claim 145, wherein the virus is:
(a) an influenza virus, optionally selected from Type A, Type B, Type C, and Type D influenza viruses, or
(b) a member of the Coronaviridae family, optionally selected from
(i) a betacoronavirus, optionally selected from Severe acute respiratory syndrome coronavirus 2 (SARS-CoV-2), SARS-CoV, Middle East Respiratory Syndrome-Corona Virus (MERS-CoV), HCoV-HKU1, and HCoV-OC43 or
(ii) an alphacoronavirus, optionally selected from HCoV-NL63 and HCoV-229E.

147. The method of claim 146, wherein the virus is SARS-CoV-2.

148. The method of any one of claims 144-147, wherein the virus is SARS-CoV-2, which has caused COVID-19.

149. The method of claim 148, wherein the COVID-19 is characterized by one or more of fever, cough, shortness of breath, diarrhea, upper respiratory symptoms, lower respiratory symptoms, pneumonia, and respiratory distress.

150. The method of any one of claims 144-149, wherein the therapy prevents or mitigates development of the acute respiratory distress syndrome (ARDS) in the patient when administered.

151. The method of any one of claims 144-150, wherein the therapy improves oxygenation in the patient when administered.

152. The method of any one of claims 144-151, wherein the therapy prevents or mitigates a transition from respiratory distress to cytokine imbalance in the patient when administered

153. The method of any one of claims 144-152, wherein the therapy reverses or prevents a cytokine storm in the patient when administered.

154. The method of any one of claims 124-153, wherein the therapy reverses or prevents a cytokine storm in the lungs or systemically in the patient when administered.

155. The method of claim 153 or 154, wherein the cytokine storm is selected from one or more of systemic inflammatory response syndrome, cytokine release syndrome, macrophage activation syndrome, and hemophagocytic lymphohistiocytosis.

156. The method of any one of claims 153-155, wherein the therapy reverses or prevents excessive production of one or more inflammatory cytokines in the patient when administered.

157. The method of claim 156, wherein the inflammatory cytokine is one or more of IL-6, IL-1, IL-1 receptor antagonist (IL-1ra), IL-2ra, IL-10, IL-18, TNFα, interferon-γ, CXCL10, and CCL7.

158. The method of any one of claims 124-157, wherein the composition is formulated for one or more of intrathecal, intra-lesional, intra-coronary, intravenous (IV), intra-articular, intramuscular, and intra-endobronchial administration and administration via intrapancreatic endovascular injection, intra-nucleus pulposus, lumbar puncture, intra-myocardium, transendocardium, intra-fistula tract, intermedullary space, intradural space and leg injection.

159. The method of any one of claims 124-158, wherein the composition is formulated for infusion.

160. The method of any one of claims 124-159, wherein the protein secretion signature is increased secretion of about 5, or about 10, or about 15, or about 20, or about 25, or about 30, or about 35, or all of MIP-1 alpha, SDF-1 alpha, IL-27, LIF, IL-1 beta, IL-2, IL-5, IL-12p70, IL-13, IL-17A, IL-31, G-CSF/CSF-3, IFN-gamma, TNF-alpha, HGF, MCP-1, IL-9, bNGF, MIP-3 alpha, Gro-alpha/KC, IL-1alpha, IL-23, MMP-1, IL-18, M-CSF, IL-21, M-CSF, IL-21, CD40L, IL-22, VEGF-A, BLC, Tweak, ENA-78 (LIX), MCP-3, MIF, and Eotaxin-3.

161. The method of any one of claims 124-160, wherein the protein secretion signature is decreased secretion of IL-6, IL-8, and/or IL-4.

162. The method of any one of claims 124-161, wherein the protein secretion signature is
(a) increased secretion of about 5, or about 10, or about 15, or about 20, or about 25, or about 30, or about 35, or all of MIP-1 alpha, SDF-1 alpha, IL-27, LIF, IL-1 beta, IL-2, IL-5, IL-12p70, IL-13, IL-17A, IL-31, G-CSF/CSF-3, IFN-gamma, TNF-alpha, HGF, MCP-1, IL-9, bNGF, MIP-3 alpha, Gro-alpha/KC, IL-1alpha, IL-23, MMP-1, IL-18, M-CSF, IL-21, M-CSF, IL-21, CD40L, IL-22, VEGF-A, BLC, Tweak, ENA-78 (LIX), MCP-3, MIF, and Eotaxin-3 and
(b) decreased secretion of IL-6, IL-8, and/or IL-4.

163. The method of any one of claims 124-162, wherein the protein secretion signature is secretion of one, or two, or three, or four, or five, or six, or all of MIP-1 alpha, G-CSF/CSF-3, M-CSF, BLC, Tweak, ENA-78 (LIX), and MCP-3.

164. The method of any one of claims 124-163, wherein the protein secretion signature is decreased secretion, relative to the IL-6 secretion by a bone marrow-derived MSC, of about 5, or about 10, or about 15, or about 20, or about 25, or about 30, or all of MIP-1 alpha, IL-27, LIF, IL-1 beta, IL-2, IL-5, IL-12p70, IL-13, IL-17A, IL-31, G-CSF/CSF-3, IFN-gamma, TNF-alpha, HGF, IL-9, bNGF, MIP-3 alpha, Gro-alpha/KC, IL-1alpha, IL-23, IL-18, M-CSF, IL-21, M-CSF, IL-21, CD40L, IL-22, BLC, ENA-78 (LIX), MCP-3, MIF, and Eotaxin-3.

165. A method of treating cancer, comprising:
(a) obtaining a mesenchymal stem cell (MSC),
the MSC having been obtained from reprogramming an induced pluripotent stem cell (iPSC), the reprogramming comprising contacting the iPSC with one or more synthetic RNA molecules encoding a reprogramming factor and
having a protein secretion signature, the protein secretion signature comprising an increased secretion of one or more proteins selected from MIP-1 alpha, SDF-1 alpha, IL-27, LIF, IL-1 beta, IL-2, IL-5, IL-12p70, IL-13, IL-17A, IL-31, G-CSF/CSF-3, IFN-gamma, TNF-alpha, HGF, MCP-1, IL-9, bNGF, MIP-3 alpha, Gro-alpha/KC, IL-1alpha, IL-23, MMP-1, IL-18, M-CSF, IL-21, M-CSF, IL-21, CD40L, IL-22, VEGF-A, BLC, Tweak, ENA-78 (LIX), MCP-3, MIF, and Eotaxin-3 and/or a decreased secretion of one or more proteins selected from IL-6, IL-8, and IL-4, wherein the increased and/or decreased secretion is relative to a bone marrow-derived MSC; and
(b) administering an effective amount of the MSC substantially having the protein secretion signature for therapy to a patient in need thereof.

166. The method of claim 165, wherein the iPSC is derived from a human.

167. The method of claim 165, wherein the iPSC is derived from a subject who is not intended to receive the therapy.

168. The method of any one of claims 165-167, wherein the iPSC is allogeneic to the patient intended to receive the therapy.

169. The method of any one of claims 165-168, wherein the iPSC is from a master cell bank.

170. The method of any one of claims 165-169, wherein the MSC is characterized by low or reduced inflammation.

171. The method of any one of claims 165-170, wherein the MSC is characterized by low or reduced immunogenicity.

172. The method of any one of claims 165-171, wherein the MSC is self-renewing.

173. The method of any one of claims 165-172, wherein the MSC is multipotent.

174. The method of any one of claims 165-173, wherein the MSC is immune inhibitory.

175. The method of any one of claims 165-174, wherein the MSC reduces the proliferation, quantity, and/or activity of an immune cell, optionally selected from a T cell and NK cell.

176. The method of any one of claims 165-175, wherein the MSC is suitable for in vitro expansion without substantial loss of immunosuppressive properties.

177. The method of any one of claims 165-176, wherein the MSC substantially expresses one or more of CD73, CD90, and CD105.

178. The method of any one of claims 165-177, wherein the MSC substantially does not express one or more of CD14, CD34 and CD45.

179. The method of any one of claims 165-178, wherein the synthetic RNA molecule is mRNA.

180. The method of claim 179, wherein the synthetic RNA molecule is mRNA comprising one or more non-canonical nucleotides selected from 2-thiouridine, 5-azauridine, pseudouridine, 4-thiouridine, 5-methyluridine, 5-methylpseudouridine, 5-aminouridine, 5-aminopseudouridine, 5-hydroxyuridine, 5-hydroxypseudouridine, 5-methoxyuridine, 5-methoxypseudouridine, 5-ethoxyuridine, 5-ethoxypseudouridine, 5-hydroxymethyluridine, 5-hydroxymethylpseudouridine, 5-carboxyuridine, 5-carboxypseudouridine, 5-formyluridine, 5-formylpseudouridine, 5-methyl-5-azauridine, 5-amino-5-azauridine, 5-hydroxy-5-azauridine, 5-methylpseudouridine, 5-aminopseudouridine, 5-hydroxypseudouridine, 4-thio-5-azauridine, 4-thiopseudouridine, 4-thio-5-methyluridine, 4-thio-5-aminouridine, 4-thio-5-hydroxyuridine, 4-thio-5-methyl-5-azauridine, 4-thio-5-amino-5-azauridine, 4-thio-5-hydroxy-5-azauridine, 4-thio-5-methylpseudouridine, 4-thio-5-aminopseudouridine, 4-thio-5-hydroxypseudouridine, 2-thiocytidine, 5-azacytidine, pseudoisocytidine, N4-methylcytidine, N4-aminocytidine, N4-hydroxycytidine, 5-methylcytidine, 5-aminocytidine, 5-hydroxycytidine, 5-methoxycytidine, 5-ethoxycytidine, 5-hydroxymethylcytidine, 5-carboxycytidine, 5-formylcytydine, 5-methyl-5-azacytidine, 5-amino-5-azacytidine, 5-hydroxy-5-azacytidine, 5-methylpseudoisocytidine, 5-aminopseudoisocytidine, 5-hydroxypseudoisocytidine, N4-methyl-5-azacytidine, N4-methylpseudoisocytidine, 2-thio-5-azacytidine, 2-thiopseudoisocytidine, 2-thio-N4-methylcytidine, 2-thio-N4-aminocytidine, 2-thio-N4-hydroxycytidine, 2-thio-5-methylcytidine, 2-thio-5-aminocytidine, 2-thio-5-hydroxycytidine, 2-thio-5-methyl-5-azacytidine, 2-thio-5-amino-5-azacytidine, 2-thio-5-hydroxy-5-azacytidine, 2-thio-5-methylpseudoisocytidine, 2-thio-5-aminopseudoisocytidine, 2-thio-5-hydroxypseudoisocytidine, 2-thio-N4-methyl-5-azacytidine, 2-thio-N4-methylpseudoisocytidine, N4-methyl-5-methylcytidine, N4-methyl-5-aminocytidine, N4-methyl-5-hydroxycytidine, N4-methyl-5-methyl-5-azacytidine, N4-methyl-5-amino-5-azacytidine, N4-methyl-5-hydroxy-5-azacytidine, N4-methyl-5-methylpseudoisocytidine, N4-methyl-5-aminopseudoisocytidine, N4-methyl-5-hydroxypseudoisocytidine, N4-amino-5-azacytidine, N4-aminopseudoisocytidine, N4-amino-5-methylcytidine, N4-amino-5-aminocytidine, N4-amino-5-hydroxycytidine, N4-amino-5-methyl-5-azacytidine, N4-amino-5-amino-5-azacytidine, N4-amino-5-hydroxy-5-azacytidine, N4-amino-5-methylpseudoisocytidine, N4-amino-5-aminopseudoisocytidine, N4-amino-5-hydroxypseudoisocytidine, N4-hydroxy-5-azacytidine, N4-hydroxypseudoisocytidine, N4-hydroxy-5-methylcytidine, N4-hydroxy-5-aminocytidine, N4-hydroxy-5-hydroxycytidine, N4-hydroxy-5-methyl-5-azacytidine, N4-hydroxy-5-amino-5-azacytidine, N4-hydroxy-5-hydroxy-5-azacytidine, N4-hydroxy-5-methylpseudoisocytidine, N4-hydroxy-5-aminopseudoisocytidine, N4-hydroxy-5-hydroxypseudoisocytidine, 2-thio-N4-methyl-5-methylcytidine, 2-thio-N4-methyl-5-aminocytidine, 2-thio-N4-methyl-5-hydroxycytidine, 2-thio-N4-methyl-5-methyl-5-azacytidine, 2-thio-N4-methyl-5-amino-5-azacytidine, 2-thio-N4-methyl-5-hydroxy-5-azacytidine, 2-thio-N4-methyl-5-methylpseudoisocytidine, 2-thio-N4-methyl-5-aminopseudoisocytidine, 2-thio-N4-methyl-5-hydroxypseudoisocytidine, 2-thio-N4-amino-5-azacytidine, 2-thio-N4-aminopseudoisocytidine, 2-thio-N4-amino-5-methylcytidine, 2-thio-N4-amino-5-aminocytidine, 2-thio-N4-amino-5-hydroxycytidine, 2-thio-N4-amino-5-methyl-5-azacytidine, 2-thio-N4-amino-5-amino-5-azacytidine, 2-thio-N4-amino-5-hydroxy-5-azacytidine, 2-thio-N4-amino-5-methylpseudoisocytidine, 2-thio-N4-amino-5-aminopseudoisocytidine, 2-thio-N4-amino-5-hydroxypseudoisocytidine, 2-thio-N4-hydroxy-5-azacytidine, 2-thio-N4-hydroxypseudoisocytidine, 2-thio-N4-hydroxy-5-methylcytidine, N4-hydroxy-5-aminocytidine, 2-thio-N4-hydroxy-5-hydroxycytidine, 2-thio-N4-hydroxy-5-methyl-5-azacytidine, 2-thio-N4-hydroxy-5-amino-5-azacytidine, 2-thio-N4-hydroxy-5-hydroxy-5-azacytidine, 2-thio-N4-hydroxy-5-methylpseudoisocytidine, 2-thio-N4-hydroxy-5-aminopseudoisocytidine, 2-thio-N4-hydroxy-5-hydroxypseudoisocytidine, N6-methyladenosine, N6-aminoadenosine, N6-hydroxyadenosine, 7-deazaadenosine, 8-azaadenosine, N6-methyl-7-deazaadenosine, N6-methyl-8-azaadenosine, 7-deaza-8-azaadenosine, N6-methyl-7-deaza-8-azaadenosine, N6-amino-7-deazaadenosine, N6-amino-8-azaadenosine, N6-amino-7-deaza-8-azaadenosine, N6-hydroxyadenosine, N6-hydroxy-7-deazaadenosine, N6-hydroxy-8-azaadenosine, N6-hydroxy-7-deaza-8-azaadenosine, 6-thioguanosine, 7-deazaguanosine, 8-azaguanosine, 6-thio-7-deazaguanosine, 6-thio-8-azaguanosine, 7-deaza-8-azaguanosine, and 6-thio-7-deaza-8-azaguanosine.

181. The method of any one of claims 165-180, wherein the synthetic RNA molecule is in vitro transcribed.

182. The method of any one of claims 165-181, wherein the reprogramming is non-viral.

183. The method of any one of claims 165-182, wherein the reprogramming factor is one or more of Oct4, Sox2, Klf4, c-Myc, I-Myc, Tert, Nanog, and Lin28.

184. The method of any one of claims 165-182, wherein the MSC is further engineered to express and/or secrete a soluble protein.

185. The method of claim 183, wherein the soluble protein is a TNF family receptor ligand, optionally TRAIL/TNFSF10 or TNFα.

186. The method of claim 183, wherein the soluble protein is an interleukin, optionally selected from IL-2, IL-6, IL-7, IL-12, IL-15, IL-18, and IL-21.

187. The method of claim 183, wherein the soluble protein is Flt-3 ligand.

188. The method of any one of claims 165-187, wherein the cancer is one or more of basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema, and Meigs' syndrome
